SEQUENCE LISTING (108 sequences)

SEQ ID NO: 1 - protein, 191 aa, Arabidopsis thaliana
  thale cress PYR/PYL receptor, Pyrabactin resistance 1, abscisic acid receptor PYR1 (PYR1), ABI1-binding protein 6 (ABIP6), regulatory components of ABA receptor 11 (RCAR11),At4g17870, T6K21.50
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 2 - protein, 221 aa, Arabidopsis thaliana
  thale cress PYR/PYL receptor, abscisic acid receptor PYL1, PYR1-like protein 1 (PYL1), ABI1-binding protein 6 (ABIP6), regulatory components of ABA receptor 9 (RCAR12), At5g46790, MZA15.21
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 3 - protein, 190 aa, Arabidopsis thaliana
  thale cress PYR/PYL receptor, abscisic acid receptor PYL2, PYR1-like protein 2 (PYL2), ABI1-binding protein 6 (ABIP6), regulatory components of ABA receptor 14 (RCAR14), Bet v I allergen family protein, At2g26040, T19L18.15
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 4 - protein, 209 aa, Arabidopsis thaliana
  thale cress PYR/PYL receptor, abscisic acid receptor PYL3, PYR1-like protein 3 (PYL3), regulatory components of ABA receptor 13 (RCAR13), At1g73000, F3N23.20
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 5 - protein, 207 aa, Arabidopsis thaliana
  thale cress PYR/PYL receptor, abscisic acid receptor PYL4, PYR1-like protein 4 (PYL4), ABI1-binding protein 2 (ABIP2), regulatory components of ABA receptor 10 (RCAR10), At2g38310, T19C21.20
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 6 - protein, 203 aa, Arabidopsis thaliana
  thale cress PYR/PYL receptor, abscisic acid receptor PYL5, PYR1-like protein 5 (PYL5), ABI1-binding protein 3 (ABIP3), regulatory components of ABA receptor 8 (RCAR8), Bet v I allergen family protein, At5g05440, K18I23.25
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 7 - protein, 215 aa, Arabidopsis thaliana
  thale cress PYR/PYL receptor, abscisic acid receptor PYL6, PYR1-like protein 6 (PYL6), ABI1-binding protein 5 (ABIP5), regulatory components of ABA receptor 9 (RCAR9), Bet v I allergen family protein, At2g40330, T7M7.15
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 8 - protein, 211 aa, Arabidopsis thaliana
  thale cress PYR/PYL receptor, abscisic acid receptor PYL7, PYR1-like protein 7 (PYL7), ABI1-binding protein 7 (ABIP7), regulatory components of ABA receptor 2 (RCAR2), At4g01026
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 9 - protein, 188 aa, Arabidopsis thaliana
  thale cress PYR/PYL receptor, abscisic acid receptor PYL8, PYR1-like protein 8 (PYL8), ABI1-binding protein 1 (ABIP1), regulatory components of ABA receptor 3 (RCAR3), At5g53160, MFH8.10
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 10 - protein, 187 aa, Arabidopsis thaliana
  thale cress PYR/PYL receptor, abscisic acid receptor PYL9, PYR1-like protein 9 (PYL9), ABI1-binding protein 4 (ABIP4), regulatory components of ABA receptor 1 (RCAR1), At1g01360, F6F3.16
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 11 - protein, 183 aa, Arabidopsis thaliana
  thale cress PYR/PYL receptor, abscisic acid receptor PYL10, PYR1-like protein 10 (PYL10), ABI1-binding protein 8 (ABIP8), regulatory components of ABA receptor 4 (RCAR4), At4g27920, T13J8.30
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 12 - protein, 161 aa, Arabidopsis thaliana
  thale cress PYR/PYL receptor, abscisic acid receptor PYL11, PYR1-like protein 11 (PYL11), regulatory components of ABA receptor 5 (RCAR5), Bet v I allergen family protein, At5g45860, K15I22.6
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLKL

SEQ ID NO: 13 - protein, 159 aa, Arabidopsis thaliana
  thale cress PYR/PYL receptor, abscisic acid receptor PYL12, PYR1-like protein 12 (PYL12), regulatory components of ABA receptor 6 (RCAR6), Bet v I allergen family protein, At5g45870, K15I22.7
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 14 - protein, 164 aa, Arabidopsis thaliana
  thale cress PYR/PYL receptor, abscisic acid receptor PYL13, PYR1-like protein 13 (PYL13), regulatory components of ABA receptor 7 (RCAR7), At4g18620, F28A21.30
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVDNIIRYNLTSLAKLTKKMMK

SEQ ID NO: 15 - protein, 191 aa, Brassica oleracea
  wild cabbage Streptomyces cyclase/dehydrase family protein, locus tag 40.t00062, GenBank Accession No. ABD65175.1, GI89257688
MPSQLTPEERSELAQSIAEFHTYHLGPGSCSSLHAQRIHAPPEIVWSVVRRFDKPQTYKHFIKSCSVEDGFEMRVGCTRAVNVISGLPANTSTERLDILDDERRVTGFSIIGGEHRLTNYKSVTTVHRFEKERRIWTVVLESYVVDMPEGNSEDDTRMFADTVVKLNLQKLATVTEAMARNAGDGSGAQVT

SEQ ID NO: 16 - protein, 281 aa, Brassica oleracea
  wild cabbage Streptomyces cyclase/dehydrase family protein, locus tag 23.t00047, GenBank Accession No. ABD65631.1, GI89274227
MPSELTQEERSKLTQSISEFHTYHLGPGSCSSLHAQRIHAPPEIVWSVVRQFDKPQTYKHFIKSCSVEEGFEMRVGCTRDVIVISGLPANTSTERLDMLDDERRVTGFSIIGGEHRLKNYKSVTTVHRFERERRIWTVVLESYVVDMPEGNSEDDTRMFADTVVKLNLQKLATVTEAMARNAGDGRGSRETTCRESFHLITAFEKQRQITEPTVYQNPPYHTGMTPEPRTSTVFIELEDHRTLPGNLTPTTEEHLQRMYQRFWGIRQLQRPRQSFGERQSI

SEQ ID NO: 17 - protein, 453 aa, Vitis vinifera
  wine grape cultivar PN40024 unnamed protein product, locus tag GSVIVT00015766001, GenBank Accession No. CAO63410.1, GI157341954
MQMKYLEGKQNLMEEKGEKQCIPMDLAVREAQFKGSLLDRITWLEQRLHKLSLQLETRSKQQPHPSRMQTAGETSSRHGPKKELSCSFPVFSTRNHNHGHKQTSQFHVPRFEYQEGGRENPAVVITKLTPFHHPKIITILFPISNYFIIFFFLTFDTKKQYPLLFPILPSRFLPISHLITQEIEKYKTSSHFSSPASLFAAMNKAETSSMAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTNHGGEIWTVVLESYVVDMPEGNTEEDTRLFADTVVKLNLQKLASVTEVSQSCNYPCQFHIIENEDIQPEEMNLGVLTTSIEEQRKKKRVVAMKDGSTSS

SEQ ID NO: 18 - protein, 195 aa, Vitis vinifera
  wine grape cultivar Pinot Noir hypothetical protein, clone ENTAV 115, locus tag VITISV_033963, GenBank Accession No. CAN64657.1, GI147789129
  MOD_RES (193)..(193): Any amino acid
MAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTVHEYQNHGGEIWTVVLESYVVDMPEGNTEEDTRLFADTVVKLNLSEAXRR

SEQ ID NO: 19 - protein, 217 aa, Medicago truncatula
  barrel medic unknown protein, clone MTYFD_FE_FF_FG1G-N-24, GenBank Accession No. ACJ85026.1, GI217073334
MEKAESSTASTSDQDSDENHRTQHHLTLPSGLRQHEFDSLIPFINSHHTYLIGPNQCSTLLAQRIHAPPQTVWSVVRSFDKPQIYKHIIKSCSLKEGFQMKVGCTRDVNVISGLPAATSTERLDVLDDERRVTGFSIIGGEHRLKNYRSVTSVHGFGDGDNGGEIWTVVLESYVVDVPEGNTEEDTRLFADTVVKLNLQKLASVTEGKNRDGDGKSH

SEQ ID NO: 20 - protein, 212 aa, Oryza sativa
  rice Japonica Group, cultivar Nipponbare, conserved hypothetical protein Os10g0573400, GenBank Accession No. NP_00106570.1, GI115483600
MEQQEEVPPPPAGLGLTAEEYAQVRATVEAHHRYAVGPGQCSSLLAQRIHAPPAAVWAVVRRFDCPQVYKHFIRSCVLRPDPHHDDNGNDLRPGRLREVSVISGLPASTSTERLDLLDDAHRVFGFTITGGEHRLRNYRSVTTVSQLDEICTLVLESYIVDVPDGNTEDDTRLFADTVIRLNLQKLKSVSEANANAAAAAAAPPPPPPAAAE

SEQ ID NO: 21 - protein, 212 aa, Zea mays
  maize cyclase/dehydrase family protein, clone 306819, GenBank Accession No. ACG40002.1, GI195641068
MDQQGAGGDAEVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELAVPAICTVVLESYVVDVPDGNTEDDTRLFADTVIRLNLQKLKSVAEANAAEAAATTNSVLLPRPAE

SEQ ID NO: 22 - protein, 212 aa, Zea mays
  maize cyclase/dehydrase family protein, clone 241996, GenBank Accession No. ACG34473.1, GI195625286
  MOD_RES (11)..(11): Any amino acid
MDQQGAGGDAXVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELADPAICTVVLESYVVDVPDGNTEDDTRLFADTVIRLNLQKLKSVTEANAAEAAATTNSVLLPRPAE

SEQ ID NO: 23 - protein, 233 aa, Vitis vinifera
  wine grape cultivar PN40024 unnamed protein product, locus tag GSVIVT00032173001, GenBank Accession No. CAO43790.1, GI157339249
MDPHHHHGLTEEEFRALEPIIQNYHTFEPSPNTCTSLITQKIDAPAQVVWPFVRSFENPQKYKHFIKDCTMRGDGGVGSIREVTVVSGLPASTSTERLEILDDEKHILSFRVVGGEHRLNNYRSVTSVNDFSKEGKDYTIVLESYIVDIPEGNTGEDTKMFVDTVVKLNLQKLAVVAITSLHENEEIADNEGPSREISLQSETESAERGDERRDGDGPSKACNRNEWHCTTKE

SEQ ID NO: 24 - protein, 207 aa, Oryza sativa
  rice Japonica Group, cultivar Nipponbare, Bet v I allergen-like protein, gene P0495C02.29, clone P0495C02, GenBank Accession No. BAD25659.1, GI49388537
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 25 - protein, 210 aa, Oryza sativa
  rice Indica Group, cultivar 93-11, hypothetical protein OsI_06433, old locus tag OsI_006310, GLEAN gene, GenBank Accession No. EAY85077.1, GI125538682
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPSPPRPYCVVVESYVVDVPEGNTEEDTRMFTDTVVKLNLQKLAAVATSSSPPAAGNHH

SEQ ID NO: 26 - protein, 200 aa, Zea mays
  maize strain B73 unknown protein, clone ZM_BFb0151H07, GenBank Accession No. ACF82013.1, GI194695858
MPYTAPRPSPQQHSRVLSGGGAKAASHGASCAAVPAEVARHHEHAARAGQCCSAVVQAIAAPVGAVWSVVRRFDRPQAYKHFIRSCRLVGGGDVAVGSVREVRVVSGLPATSSRERLEILDDERRVLSFRVVGGEHRLANYRSVTTVHEAGAGAGTGTVVVESYVVDVPHGNTADETRVFVDTIVRCNLQSLARTAERLA

SEQ ID NO: 27 - protein, 215 aa, Vitis vinifera
  wine grape cultivar PN40024 unnamed protein product, locus tag GSVIVT00037390001, GenBank Accession No. CAO48777.1, GI157355387
MPSNPPKSSLVVHRINSPNSITTATTASAAANNHNTSTMPPHKQVPDAVSRHHTHVVGPNQCCSAVVQQIAAPVSTVWSVVRRFDNPQAYKHFVKSCHVVVGDGDVGTLREVHVISGLPAANSTERLEILDDERHVLSFSVIGGDHRLSNYRSVTTLHPSPSSTGTVVLESYVVDIPPGNTKEDTCVFVDTIVRCNLQSLAQIAENAAGCKRSSS

SEQ ID NO: 28 - protein, 213 aa, Nicotiana tabacum
  tobacco hypothetical protein, gene c17, GenBank Accession No. CAI84653.1, GI62867576
MPPSSPDSSVLLQRISSNTTPDFACKQSQQLQRRTMPIPCTTQVPDSVVRFHTHPVGPNQCCSAVIQRISAPVSTVWSVVRRFDNPQAYKHFVKSCHVIVGDGDVGTLREVRVISGLPAASSTERLEILDDERHVISFSVVGGDHRLANYRSVTTLHPEPSGDGTTIVVESYVVDVPPGNTRDETCVFVDTIVKCNLTSLSQIAVNVNRRKDS

SEQ ID NO: 29 - protein, 208 aa, Oryza sativa
  rice Indica Group, cultivar 93-11, hypothetical protein OsI_04285, old locus tag OsI_004197, GLEAN gene, GenBank Accession No. EAY76350.1, GI125528236
MPYAAVRPSPPPQLSRPIGSGAGGGKACPAVPCEVARYHEHAVGAGQCCSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 30 - protein, 208 aa, Oryza sativa
  rice Japonica Group, cultivar Nipponbare, Bet v I allergen-like protein, gene B1088C09.11, clone B1088C09, GenBank Accession No. BAB68102.1, GI15624049
MPYAAVRPSPPPQLSRPIGSGAGGGKACPAVPCEVARYHEHAVGAGQCFSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 31 - protein, 213 aa, Picea sitchensis
  Sitka spruce cultivar FB3-425, unknown protein, clone WS0276_P02, GenBank Accession No. ABK22940.1, GI116783434
MDIIAGFDQLSFRLSGASKQITKTGAVQYLKGEEGYGEWLKEVMGRYHYHSHDGARECRCSSVVVQQVEAPVSVVWSLVRRFDQPQVYKHFVSNCFMRGDLKVGCLREVRVVSGLPAATSTERLDILDEERHILSFSIVGGDHRLNNYRSITTLHETLINGKPGTIVIESYVLDVPHGNTKEETCLFVDTIVKCNLQSLAHVSNHLNSTHRCL

SEQ ID NO: 32 - protein, 207 aa, Oryza sativa
  rice Japonica Group, cultivar Nipponbare, hypothetical protein Os06g0562200, Bet v I allergen family protein, GenBank Accession No. NP_001057874.1, GI115468550
MEAHVERALREGLTEEERAALEPAVMAHHTFPPSTTTATTAAATCTSLVTQRVAAPVRAVWPIVRSFGNPQRYKHFVRTCALAAGDGASVGSVREVTVVSGLPASTSTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 33 - protein, 216 aa, Oryza sativa
  rice Japonica Group, cultivar Nipponbare, hypothetical protein Os05g0473000, Streptomyces cyclase/dehydrase family protein, GenBank Accession No. NP_001055819.1, GI115464439
MPYTAPRPSPPQHSRIGGCGGGGVLKAAGAAGHAASCVAVPAEVARHHEHAAGVGQCCSAVVQAIAAPVDAVWSVVRRFDRPQAYKHFIRSCRLLDGDGDGGAVAVGSVREVRVVSGLPATSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 34 - protein, 212 aa, Vitis vinifera
  wine grape cultivar PN40024 unnamed protein product, locus tag GSVIVT00029365001, GenBank Accession No. CAO41436.1, GI157351249
MPSSLQLHRINNIDPTTVAVAATAAVNCHKQSRTPLRCATPVPDAVASYHAHAVGPHQCCSMVVQTTAAALPTVWSVVRRFDNPQAYKHFLKSCHVIFGDGDIGTLREVHVVSGLPAESSTERLEILDDERHVLSFSVVGGDHRLCNYRSVTTLHPSPTGTGTVVVESYVVDIPPGNTKEDTCVFVDTIVKCNLQSLAQMSEKLTNNNRNSS

SEQ ID NO: 35 - protein, 218 aa, Zea mays
  maize cyclase/dehydrase family protein, clone 1678999, GenBank Accession No. ACG30334.1, GI195617008
MPCLQASSPGSMPYQHHGRGVGCAAEAGAAVGASAGTGTRCGAHDGEVPAEAARHHEHAAPGPGRCCSAVVQRVAAPAEAVWSVVRRFDQPQAYKRFVRSCALLAGDGGVGTLREVRVVSGLPAASSRERLEVLDDESHVLSFRVVGGEHRLQNYLSVTTVHPSPAAPDAATVVVESYVVDVPPGNTPEDTRVFVDTIVKCNLQSLATTAEKLALAAV

SEQ ID NO: 36 - protein, 179 aa, Physcomitrella patens
  Physcomitrella patens subsp. patens bryophyte moss, ecotype Gransden 2004, hypothetical protein, predicted protein, locus tag PHYPADRAFT_222359, GenBank Accession No. XP_001778048.1, GI168051209
MQTKGRQADFQTLLEGQQDLICRFHRHELQPHQCGSILLQLIKAPVETVWSVARSFDKPQVYKRFIQTCEIIEGDGGVGSIREVRLVSSIPATSSIERLEILDDEEHIISFRVLGGGHRLQNYWSVTSLHSHEIDGQMGTLVLESYVVDIPEGNTREETHMFVDTVVRCNLKALAQVSE

SEQ ID NO: 37 - protein, 229 aa, Oryza sativa
  rice Indica Group, cultivar 93-11, hypothetical protein OsI_11160, old locus tag OsI_010864, GLEAN gene, GenBank Accession No. EAY89631.1, GI125543492
MPCIPASSPGIPHQHQHQHHRALAGVGMAVGCAAEAAVAAAGVAGTRCGAHDGEVPMEVARHHEHAEPGSGRCCSAVVQHVAAPAPAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 38 - protein, 229 aa, Oryza sativa
  rice Japonica Group, cultivar Nipponbare, hypothetical protein Os03g0297600, Streptomyces cyclase/dehydrase family protein, GenBank Accession No. NP_001049838.1, GI115452475
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 39 - protein, 205 aa, Medicago truncatula
  barrel medic unknown protein, clone MTYFP_FQ_FR_FS1G-H-19, GenBank Accession No. ACJ85898.1, GI217075076
MPSPVQFQRFDSNTAITNGVNCPKQIQACRYALSSLKPTVSVPETVVDHHMHVVGQNQCYSVVIQTINASVSTVWSVVRRFDYPQGYKHFVKSCNVVASGDGIRVGALREVRLVSGLPAVSSTERLDILDEERHVISFSVVGGVHRCRNYRSVTTLHGDGNGGTVVIESYVVDVPQGNTKEETCSFADTIVRCNLQSLVQIAEKL

SEQ ID NO: 40 - protein, 212 aa, Zea mays
  maize AT-rich element binding factor 3, clone 1458362, GenBank Accession No. ACG26321.1, GI195608982
MPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 41 - protein, 233 aa, Zea mays
  maize strain B73 unknown protein, clone ZM_BFb0105O18, GenBank Accession No. ACF87013.1, GI194705858
MRERNSSIDQEHQRGSSSRSTMPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 42 - protein, 194 aa, Physcomitrella patens
  Physcomitrella patens subsp. patens bryophyte moss, ecotype Gransden 2004, hypothetical protein, predicted protein, locus tag PHYPADRAFT_209242, GenBank Accession No. XP_001762113.1, GI168019160
MMQEKQGRPDFQFLLEGQQDLICRFHKHELLPHQCGSILLQQIKAPVQTVWLIVRRFDEPQVYKRFIQRCDIVEGDGVVGSIREVQLVSSIPATSSIERLEILDDEEHIISFRVLGGGHRLQNYWSVTSLHRHEIQGQMGTLVLESYVVDIPDGNTREETHTFVDTVVRCNLKALAQVSEQKHLLNSNEKPAAP

SEQ ID NO: 43 - protein, 191 aa, Vitis vinifera
  wine grape cultivar PN40024 unnamed protein product, locus tag GSVIVT00035869001, GenBank Accession No. CAO48052.1, GI157354734
MKVYSPSQILAERGPRAQAMGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 44 - protein, 217 aa, Physcomitrella patens
  Physcomitrella patens subsp. patens bryophyte moss, ecotype Gransden 2004, hypothetical protein, predicted protein, locus tag PHYPADRAFT_132509, GenBank Accession No. XP_001767821.1, GI168030621
MQQVKGRQDFQRLLEAQQDLICRYHTHELKAHQCGSILLQQIKVPLPIVWAIVRSFDKPQVYKRFIQTCKITEGDGGVGSIREVHLVSSVPATCSIERLEILDDEKHIISFRVLGGGHRLQNYSSVSSLHELEVEGHPCTLVLESYMVDIPDGNTREETHMFVDTVVRCNLKSLAQISEQQYNKDCLQQKQHDQQQMYQQRHPPLPPIPITDKNMER

SEQ ID NO: 45 - protein, 195 aa, Physcomitrella patens
  Physcomitrella patens subsp. patens bryophyte moss, ecotype Gransden 2004, hypothetical protein, predicted protein, locus tag PHYPADRAFT_213389, GenBank Accession No. XP_001767012.1, GI168028995
MRFDIGHNDVRGFFTCEEEHAYALHSQTVELNQCGSILMQQIHAPIEVVWSIVRSFGSPQIYKKFIQACILTVGDGGVGSIREVFLVSGVPATSSIERLEILDDEKHVFSFRVLKGGHRLQNYRSVTTLHEQEVNGRQTTTVLESYVVDVPDGNTREETHMFADTVVMCNLKSLAQVAEWRAMQGITQQLSTSSL

SEQ ID NO: 46 - protein, 172 aa, Vitis vinifera
  wine grape cultivar Pinot Noir hypothetical protein, clone ENTAV 115, locus tag VITISV_004947, GenBank Accession No. CAN72620.1, GI147840019
MGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 47 - protein, 196 aa, Picea sitchensis
  Sitka spruce cultivar FB3-425, unknown protein, clone WS0281_I24, GenBank Accession No. ABK23752.1, GI116785512
MEDLSSWREGRAMWLGNPPSESELVCRHHRHELQGNQCSSFLVKHIRAPVHLVWSIVRTFDQPQKYKPFVHSCSVRGGITVGSIRNVNVKSGLPATASEERLEILDDNEHVFSIKILGGDHRLQNYSSIITVHPEIIDGRPGTLVIESYVVDVPEGNTREETRFFVEALVKCNLKSLADVSERLASQHHTELLERT

SEQ ID NO: 48 - protein, 185 aa, Solanum tuberosum
  potato cultivar Kuras, CAPIP1-like protein, clone 153D02, similar to Capsicum annuum CAPIP1, GenBank Accession No. ABB29920.1, GI78191398
MNANGFCGVEKEYIRKHHLHEPKENQCSSFLVKHIRAPVHLVWSLVRRFDQPQKYKPFISRCIVQGDLEIGSLREVDVKSGLPATTSTERLELLDDEEHILSVRIVGGDHRLRNYSSVISVHPEVIDGRPGTVVLESFVVDVPEGNTKDETCYFVEALINCNLKSLADISERVAVQDRTEPIDQV

SEQ ID NO: 49 - protein, 190 aa, Medicago truncatula
  barrel medic unknown protein, clone MTYFP_FQ_FR_FS1G-E-17, GenBank Accession No. ACJ85952.1, GI217075184
MNNGCEQQQYSVIETQYIRRHHKHDLRDNQCSSALVKHIKAPVHLVWSLVRRFDQPQKYKPFISRCIMQGDLSIGSVREVNVKSGLPATTSTERLEQLDDEEHILGIRIVGGDHRLRNYSSIITVHPGVIDGRPGTMVIESFVVDVPEGNTKDETCYFVEALIRYNLSSLADVSERMAVQGRTDPININP

SEQ ID NO: 50 - protein, 185 aa, Vitis vinifera
  wine grape cultivar PN40024 unnamed protein product, locus tag GSVIVT00002440001, GenBank Accession No. CAO65816.1, GI157358179
MSGYGCIKMEDEYIRRHHRHEIRDNQCSSSLVKHIKAPVHLVWSLVRSFDQPQKYKPFVSRCIVQGDLEIGSVREVNVKSGLPATTSTERLELLDDEEHIFGMRIVGGDHRLKNYSSIVTVHPEIIDGRPGTLVIESFVVDVPDGNTKDETCYFVEALIKCNLKSLADVSERLAIQDRTEPIDRM

SEQ ID NO: 51 - protein, 185 aa, Vitis vinifera
  wine grape cultivar PN40024 unnamed protein product, locus tag GSVIVT00006507001, GenBank Accession No. CAO69376.1, GI157360187
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFVEALIKCNLKSLADVSERLAVQDRTEPIDRM

SEQ ID NO: 52 - protein, 208 aa, Oryza sativa
  rice Japonica Group, cultivar Nipponbare, hypothetical protein OsJ_21703, old locus tag OsJ_020847, GLEAN gene, GenBank Accession No. EAZ37364.1, GI125597584
MEAHVERALREGLTEEERAALEPAVMAHHTFPPSTTTATTAAATCTSLVTQRVAAPVRAVWPIVRSFGNPQRYKHFVRTCALAAGNGPSFGSVREVTVVSGPSRLPPGTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 53 - protein, 186 aa, Capsicum annuum
  pepper cultivar hanbyul, CAPIP1 protein, GenBank Accession No. AAT35532.1, GI47558817
MMNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 54 - protein, 186 aa, Populus trichocarpa
  California poplar (Western balsam poplar, black cottonwood) cultivar 383-2499 (Nisqually-1), unknown protein, clone PX0011_1113, GenBank Accession No. ABK92491.1, GI118481075
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVEALIRCNLKSLADVSERMAVQDRVEPVNQF

SEQ ID NO: 55 - protein, 185 aa, Capsicum annuum
  pepper cultivar hanbyul, PIP1 protein, GenBank Accession No. ABF72432.1, GI104304209
MNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 56 - protein, 186 aa, Unknown
  Description of Unknown Populus trichocarpa x Populus deltoides, California poplar (Western balsam poplar, black cottonwood) x Eastern cottonwood, cultivar H11-11, unknown protein, clone
  CONT. FROM ABOVE WS0133_I04, GenBank Accession No. ABK96505.1, GI118489403
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVKALIRCNLKSLADVSERMAVQDRVEPVNQF

SEQ ID NO: 57 - protein, 188 aa, Pisum sativum
  pea AT-rich element binding factor 3 (PsATF, ATF3), potential transcription factor for PsCHS1, GenBank Accession No. AAV85853.1, GI56384584
MNNGGEQYSAIETQYIRRRHKHDLRDNQCSSALVKHIKAPVHLVWSLVRRFDQPQKYKPFVSRCIMQGDLGIGSVREVNVKSGLPATTSTERLEQLDDEEHILGIRIVGGDHRLRNYSSVITVHPEVIDGRPGTMVIESFVVDVPEGNTRDETCYFVEALIRGNLSSLADVSERMAVQGRTDPINVNP

SEQ ID NO: 58 - protein, 177 aa, Vitis vinifera
  wine grape cultivar PN40024 unnamed protein product, locus tag GSVIVT00027009001, GenBank Accession No. CAO39744.1, GI157349888
MEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPANAV

SEQ ID NO: 59 - protein, 178 aa, Vitis vinifera
  wine grape cultivar Pinot Noir hypothetical protein, clone ENTAV 115, locus tag VITISV_004915, GenBank Accession No. CAN82501.1, GI147856414
MMEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPANAV

SEQ ID NO: 60 - protein, 193 aa, Arachis hypogaea
  peanut pathogenesis-induced protein (PIP), GenBank Accession No. ACG76109.1, GI196196276
  MOD_RES (162)..(162): Any amino acid
MMNGSCGGGGGGEAYGAIEAQYIRRHHRHEPRDNQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIEGRPGTMVIESFVVDVPDGNTKDETCXFVEALIRCNLSSLADVSERMAVQGRTDPINQ

SEQ ID NO: 61 - protein, 217 aa, Zea mays
  maize AT-rich element binding factor 3, clone 300908, GenBank Accession No. ACG39386.1, GI195639836
MVVEMDGGVGVAAGGGGGAQTPAPAPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 62 - protein, 217 aa, Zea mays
  maize strain B73, unknown protein, clone ZM_BFb0036A01, GenBank Accession No. ACF80077.1, GI194691986
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 63 - protein, 206 aa, Oryza sativa
  rice Japonica Group, cultivar Nipponbare, conserved hypothetical protein Os06g0528300, GenBank Accession No. NP_001057772.1, GI115468346
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVNFIGGDHMLKNYSSILTVHSEVIDGQLGTLVVESFIVDVPEGNTKDDISYFIENVLRCNLRTLADVSEERLANP

SEQ ID NO: 64 - protein, 206 aa, Oryza sativa
  rice Indica Group, cultivar 93-11, hypothetical protein OsI_23215, old locus tag OsI_022420, GLEAN gene, GenBank Accession No. EAZ01188.1, GI125555582
MNGAGGAGGAAAGKLPMVSHRQVQWRLADERCELREEEMEYIRQFHRHEPSSNQCTSFVAKHIKAPLQTVWSLVRRFDQPQLFKPFVRKCVMRENIIATGCVREVNVQSGLPATRSTERLELLDDNEHILKVKFIGGDHMLKNYSSILTIHSEVIDGQLGTLVVESFVVDIPEGNTKDDICYFIENILRCNLMTLADVSEERLANP

SEQ ID NO: 65 - protein, 205 aa, Oryza sativa
  rice Japonica Group, cultivar Nipponbare, hypothetical protein OsJ_06125, old locus tag OsI_005939, GenBank Accession No. EAZ22456.1, GI125581525
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVCQGPNRAPSTR

SEQ ID NO: 66 - protein, 204 aa, Oryza sativa
  rice Japonica Group, cultivar Nipponbare, hypothetical protein Os02g0255500, similar to extensin (fragment), GenBank Accession No. NP_001046464.1, GI115445369
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVVKDQTEPLDR

SEQ ID NO: 67 - protein, 199 aa, Medicago truncatula
  barrel medic unknown protein, clone MTYFP_FQ_FR_FS1G-G-11, GenBank Accession No. ACJ86004.1, GI217075288
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAVQDLTEPLDRVHELLISG

SEQ ID NO: 68 - protein, 199 aa, Medicago truncatula
  barrel medic unknown protein, clone MTYF1_F2_F3_FY1G-K-4, GenBank Accession No. ACJ83958.1, GI217071196
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAAQDLTEPLDRMHELLISG

SEQ ID NO: 69 - protein, 197 aa, Zea mays
  maize CAPIP1 protein, clone 244179, GenBank Accession No. ACG34726.1, GI195625792
MVGLVGGSTARAEHVVANAGGEAEYVRRMHRHAPTEHQCTSTLVKHIKAPVHLVWQLVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 70 - protein, 197 aa, Zea mays
  maize CAPIP1 protein, clone 1448906, GenBank Accession No. ACG26022.1, GI195608384
MVGLVGGSTARAEHVVANAGGEAEYVRRMHRHAPTEHQCTSTLVKHIKAPVHLVWELVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 71 - protein, 212 aa, Zea mays
  maize strain B73 unknown protein, clone ZM_BFc0183D21, GenBank Accession No. ACF86162.1, GI194704156
MVMVEMDGGVGGGGGGGQTPAPRRWRLADERCDLRAMETDYVRRFHRHEPREHQCSSAVAKHIKAPVHLVWSLVRRFDQPQLFKPFVSRCEMKGNIEIGSVREVNVKSGLPATRSTERLELLDDNEHILSVRFVGGDHRLQNYSSILTVHPEVIDGRPGTLVIESFVVDVPDGNTKDETCYFVEALLKCNLKSLAEVSERQVVKDQTEPLDR

SEQ ID NO: 72 - protein, 205 aa, Oryza sativa
  rice Japonica Group, cultivar Nipponbare, conserved hypothetical protein Os06g0527800, GenBank Accession No. NP_001057771.1, GI115468344
MNGAGGAGGAAAGKLPMVSHRRVQCRLADKRCELREEEMEYIRQFHRHEPSSNQCTSFVAKHIKAPLQTVWSLVRRFDQPQLFKPFVRKCVMRENIIVTGCVREVNVQSGLPATRSTERLELLDDNEHILKVKFIGGDHMLKNYSSILTIHSEVIDGQLGTLVVESFVVDIPDGNTKDDICYFIENVLRCNLMTLADVSEERLAN

SEQ ID NO: 73 - protein, 197 aa, Zea mays
  maize strain B73 unknown protein, clone ZM_BFc0063E17, GenBank Accession No. ACF85073.1, GI194701978
MVGLVGGSTARAEHVVANAGGETEYVRRLHRHAPAEHQCTSTLVKHIKAPVHLVWELVRSFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLKSLAEVSEQLAVESPTSPIDQ

SEQ ID NO: 74 - protein, 206 aa, Oryza sativa
  rice Indica Group, cultivar 93-11, hypothetical protein OsI_23218, old locus tag OsI_022423, GLEAN gene, GenBank Accession No. EAZ01191.1, GI125555585
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVKFIGGDHMLKNYSSILTVHSEVIDGQLGTLVVESFIVDVLEGNTKDDISYFIENVLRCNLRTLADVSEERLANP

SEQ ID NO: 75 - protein, 209 aa, Oryza sativa
  rice Japonica Group, cultivar Nipponbare, conserved hypothetical protein Os05g0213500, GenBank Accession No. NP_001054923.1, GI115462647
MVGLVGGGGWRVGDDAAGGGGGGAVAAGAAAAAEAEHMRRLHSHAPGEHQCSSALVKHIKAPVHLVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEVSERLAVQSPTSPLEQ

SEQ ID NO: 76 - protein, 180 aa, Oryza sativa
  rice Japonica Group, cultivar Nipponbare, Bet v I allergen-like protein, clone OSJNBa0052K15, gene OSJNBa0052K15.17, GenBank Accession No. BAD29692.1, GI50251668
MVEMDAGGRPEPSPPSGQCSSAVTMRINAPVHLVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPAQ

SEQ ID NO: 77 - protein, 176 aa, Vitis vinifera
  wine grape cultivar Pinot Noir hypothetical protein, clone ENTAV 115, locus tag VITISV_029498, GenBank Accession No. CAN64668.1, GI147797548
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFSLADVSERLAVAGTVTEPIDRM

SEQ ID NO: 78 - protein, 180 aa, Oryza sativa
  rice Indica Group, cultivar 93-11, hypothetical protein, locus tag OsI_06615, GLEAN gene, GenBank Accession No. EEC72859.1, GI218190432
MVEMDAGGRPEPSPPSGQCSSAVTMRINAPVHLVWSIVRRFEEPHIFQPFVRGCTMRGSTSLAVGCVREVDFKSGFSAKSSVERLEILDDKEHVFGVRIIGGDHRLKNYSSVLTAKPEVIDGEPATLVSESFVIDVPEGNTADETRHFVEFLIRCNLRSLAMVSQRLLLAQGDLAEPPAQ

SEQ ID NO: 79 - protein, 215 aa, Oryza sativa
  rice Japonica Group, cultivar Nipponbare, hypothetical protein OsJ_10498, old locus tag OsJ_010081, GLEAN gene, GenBank Accession No. EAZ26598.1, GI125585934
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGLGKVRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 80 - protein, 186 aa, Rheum australe
  Himalayan rhubarb pathogen-induced protein-like protein, GenBank Accession No. ACH63237.1, GI197312913
MNGDGYGGSEEEFVKRYHEHVLADHQCSSVLVEHINAPLHLVWSLVRSFDQPQKYKPFVSRCVVQGGDLEIGSVREVDVKSGLPATTSMEELELLDDKEHVLRVKFVGGDHRLKNYSSIVSLHPEIIGGRSGTMVIESFIVDIADGNTKEETCYFIESLINCNLKSLSCVSERLAVEDIAERIAQM

SEQ ID NO: 81 - protein, 254 aa, Oryza sativa
  rice Japonica Group, cultivar Nipponbare, hypothetical protein, locus tag OsJ_016770, GenBank Accession No. EAZ33287.1, GI125593228
MVGLVGGGGWRVGDDAAGGGGGGAVAAGAAAAAEAEHMRRLHSQGPRRAPVQLRARQAHQGSCSPPRIECANFAVFLAARDPKIVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEMVRMISLVLPFMLVDRMSGITCESHLETTLVRCGEYAVLAHV

SEQ ID NO: 82 - protein, 186 aa, Oryza sativa
  rice Japonica Group, cultivar Nipponbare, hypothetical protein, locus tag OsJ_005784, GenBank Accession No. EAZ22301.1, GI125581370
MEPHMERALREAVASEAERRELEGVVRAHHTGWNAPLAAVWPHRARVRPTRSGTSTSSSRASSPPGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 83 - protein, 150 aa, Oryza sativa
  rice Japonica Group, cultivar Nipponbare, hypothetical protein, locus tag OsJ_005938, GenBank Accession No. EAZ22455.1, GI125581524
MEVVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPGQ

SEQ ID NO: 84 - protein, 206 aa, Oryza sativa
  rice Japonica Group, cultivar Nipponbare, hypothetical protein, locus tag OsJ_018129, GenBank Accession No. EAZ34646.1, GI125594587
MPYTAPRPSPPQHSRIGGCGGGGVLKAAGAAGHAASCVAVPAEVARHHEHAAGVGQCCSAVVQAIAAPVDAVWRTSTSSGAAASWTATATAGPLPVGSVREFRVLSGLPGTSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 85 - protein, 396 aa, Vitis vinifera
  wine grape cultivar Pinot Noir hypothetical protein, clone ENTAV 115, locus tag VITISV_001710, GenBank Accession No. CAN76441.1, GI147770961
  MOD_RES (61)..(61): Any amino acid
  MOD_RES (168)..(168): Any amino acid
  MOD_RES (181)..(181): Any amino acid
  MOD_RES (277)..(277): Any amino acid
MPISSLPFSLYTVTPNPLKLITTHAHAFTPHTHIFTLKFMSHTYCPHIHHITSIHYTHLLXPIPHMPLQPPLPPHPILPSMPAFQHLYSTNQHLQVALFSARGPNIRDFNFQDADLLKLDILAPGSLIWAAWSPNGTDEANYVGEGSPTVAMIAKRGPRHGKYMAFCXMYRDNVAPKGVNXAVATVKTKRTIQLKTSLEIACHYAGINISGINGEVMPGQWEYQVGPGQCSSLLAQRVHVPLSAVGSVVHRFDKPQRYQHVIKSCRIEDGFEMRMGXLRDVNIISGLPTATNTGRLDMQDDERHVTRCPHQRQSESKYTENNNSDASSIKSPINGPSEHLKTAASPKTESIIVIDTSKFLNEEDFEGKDETSSSNQVQIEDENWETRFPNTDAGIW

SEQ ID NO: 86 - protein, 443 aa, Vitis vinifera
  wine grape cultivar Pinot Noir hypothetical protein, clone ENTAV 115, locus tag VITISV_014403, GenBank Accession No. CAN9881.1, GI147828564
  MOD_RES (5)..(5): Any amino acid
  MOD_RES (15)..(15): Any amino acid
  MOD_RES (129)..(129): Any amino acid
  MOD_RES (220)..(220): Any amino acid
  MOD_RES (401)..(401): Any amino acid
  MOD_RES (443)..(443): Any amino acid
MPSAXKSSTVPLSLXQFKLGLRHGHRVIPWGDLDSLAMLQRQLDVDILVTGHTHRFTAYKHEGGVVINPGSATGAFGSITYDVNPSFVLMDIDGLRVVVCVYELIDETANIIKELHARKISFGTKSMIXCLLLKRRSTPKFRRKKLFLFQCRVQMTLTLTNLAVSGIAQTLQVDQWTVCALIFMTRRDIHLDKARFLDFKDMGKLLADASGLRKALSGGXVTAGMAIFDTMRHIRPDVPTVCVGLAAVAMIAKRGPRHGKYMAFCPMYRDNVAPKGVNVAVVTVKTKRTIQLKTSLEIACHYAGINISGINGEVMPGQWEYQVGPGQCSSLLAQRVHVPLSAVGSVVHRFDKPQRYQHVIKSCRIEDGFEMRMGRLRDVNIISGLPTATNTGRLDMQDDEXHVTRCPHQRQSESKYTENNNSDASSVKSPINGPSEHLKTAAX

SEQ ID NO: 87 - protein, 95 aa, Oryza sativa
  rice Indica Group, cultivar Pokkali, capip1 protein (partial), clone OSR-385-428-D5, GenBank Accession No. ABR25904.1, GI149392053
EIGSVREVNVKTGLPATTSTERLELLDDDEHILSVKFVGGDHRLRNYSSIVTVHPESIDGRPGTLVIESFVVDVPDGNTKDETCYFVEAVIKCNL

SEQ ID NO: 88 - protein, 191 aa, Zea mays
  maize strain B73 unknown protein, clone ZM_BFc0034O07, GenBank Accession No. ACF84624.1, GI194701080
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQVCSVLHLSIFCAAHARYFAHHLKCVLEFLCQMHLDVLPCDDAILE

SEQ ID NO: 89 - protein, 239 aa, Oryza sativa
  rice Japonica Group, cultivar Nipponbare, hypothetical protein, locus tag OsJ_020681, GenBank Accession No. EAZ37198.1, GI125597418
MNGCTGGAGGVAAGRLPAVSLQQAQWKLVDERCELREEEMEYVRRFHRHEIGSNQCNSFIAKHVRAPLQNVWSLVRRFDQPQIYKPFVRKCVMRGNVETGSVREIIVQSGLPATRSIERLEFLDDNEYILRVKFIGGDHMLKKRIPKKTYAISSRTCSDSAIIAVGQSNCAPEITAMNGGVSIQPWLILLAFFSSPSNQTNPDSLRDMHPGSWFQILLVLAMFTCSKGSVLPPSEKVNV

SEQ ID NO: 90 - protein, 24 aa, Artificial Sequence
  Description of Artificial Sequence Synthetic PYR/PYL polypeptide conserved motif consensus sequence
  MOD_RES (2)..(2): Any amino acid
  MOD_RES (4)..(6): Any amino acid
  MOD_RES (8)..(18): Any amino acid
  MOD_RES (21)..(21): Any amino acid
  MOD_RES (23)..(23): Any amino acid
EXLXXXDXXXXXXXXXXXGGXHXL

SEQ ID NO: 91 - protein, 36 aa, Artificial Sequence
  Description of Artificial Sequence Synthetic PYR/PYL polypeptide conserved motif consensus sequence
  MOD_RES (2)..(2): Any amino acid
  MOD_RES (4)..(10): Any amino acid
  MOD_RES (13)..(16): Any amino acid
  MOD_RES (18)..(22): Any amino acid
  MOD_RES (24)..(25): Any amino acid
  MOD_RES (27)..(31): Any amino acid
  MOD_RES (33)..(35): Any amino acid
CXSXXXXXXXAPXXXXWXXXXXFXXPXXXXXFXXXC

SEQ ID NO: 92 - protein, 25 aa, Artificial Sequence
  Description of Artificial Sequence Synthetic PYR/PYL polypeptide conserved motif consensus sequence
  MOD_RES (2)..(3): Any amino acid
  MOD_RES (5)..(5): Any amino acid
  MOD_RES (7)..(9): Any amino acid
  MOD_RES (11)..(12): Any amino acid
  MOD_RES (15)..(16): Any amino acid
  MOD_RES (18)..(18): Any amino acid
  MOD_RES (20)..(20): Any amino acid
  MOD_RES (22)..(24): Any amino acid
GXXRXVXXXSXXPAXXSXEXLXXXD

SEQ ID NO: 93 - protein, 11 aa, Artificial Sequence
  Description of Artificial Sequence Synthetic PYR/PYL polypeptide conserved motif consensus sequence
  MOD_RES (3)..(3): Any amino acid
  MOD_RES (7)..(7): Any amino acid
  MOD_RES (10)..(10): Any amino acid
GGXHRLXNYXS

SEQ ID NO: 94 - protein, 36 aa, Artificial Sequence; identical to SEQ ID NO: 91
  Description of Artificial Sequence Synthetic PYR1 to PYL12 Arabidopsis PYR/PYL polypeptide consensus sequence
  MOD_RES (2)..(2): Any amino acid
  MOD_RES (4)..(10): Any amino acid
  MOD_RES (13)..(16): Any amino acid
  MOD_RES (18)..(22): Any amino acid
  MOD_RES (24)..(25): Any amino acid
  MOD_RES (27)..(31): Any amino acid
  MOD_RES (33)..(35): Any amino acid

SEQ ID NO: 95 - protein, 25 aa, Artificial Sequence
  Description of Artificial Sequence Synthetic PYR1 to PYL12 Arabidopsis PYR/PYL polypeptide consensus sequence
  MOD_RES (2)..(3): Any amino acid
  MOD_RES (5)..(5): Any amino acid
  MOD_RES (7)..(9): Any amino acid
  MOD_RES (11)..(12): Any amino acid
  MOD_RES (15)..(16): Any amino acid
  MOD_RES (18)..(18): Any amino acid
  MOD_RES (20)..(20): Any amino acid
  MOD_RES (22)..(24): Any amino acid
GXXRXVXXXSXXPAXXSXEXLXXXD

SEQ ID NO: 96 - protein, 31 aa, Artificial Sequence
  Description of Artificial Sequence Synthetic PYR1 to PYL12 Arabidopsis PYR/PYL polypeptide consensus sequence
  MOD_RES (3)..(4): Any amino acid
  MOD_RES (7)..(7): Any amino acid
  MOD_RES (9)..(9): Any amino acid
  MOD_RES (11)..(15): Any amino acid
  MOD_RES (17)..(18): Any amino acid
  MOD_RES (20)..(26): Any amino acid
  MOD_RES (29)..(30): Any amino acid
ESXXVDXPXGXXXXXTXXFXXXXXXXNLXXL

SEQ ID NO: 97 - protein, 36 aa, Artificial Sequence
  Description of Artificial Sequence Synthetic PYL1-12 Arabidopsis PYR/PYL polypeptide consensus sequence
  MOD_RES (2)..(2): Any amino acid
  MOD_RES (4)..(10): Any amino acid
  MOD_RES (13)..(16): Any amino acid
  MOD_RES (18)..(22): Any amino acid
  MOD_RES (24)..(25): Any amino acid
  MOD_RES (27)..(29): Any amino acid
  MOD_RES (31)..(31): Any amino acid
  MOD_RES (33)..(35): Any amino acid
CXSXXXXXXXAPXXXXWXXXXXFXXPXXXKXFXXXC

SEQ ID NO: 98 - protein, 25 aa, Artificial Sequence
  Description of Artificial Sequence Synthetic PYL1-12 Arabidopsis PYR/PYL polypeptide consensus sequence
  MOD_RES (2)..(3): Any amino acid
  MOD_RES (5)..(5): Any amino acid
  MOD_RES (7)..(9): Any amino acid
  MOD_RES (11)..(11): Any amino acid
  MOD_RES (15)..(16): Any amino acid
  MOD_RES (18)..(18): Any amino acid
  MOD_RES (20)..(20): Any amino acid
  MOD_RES (22)..(24): Any amino acid
GXXRXVXXXSXLPAXXSXEXLXXXD

SEQ ID NO: 99 - protein, 31 aa, Artificial Sequence
  Description of Artificial Sequence Synthetic PYL1-12 Arabidopsis PYR/PYL polypeptide consensus sequence
  MOD_RES (3)..(4): Any amino acid
  MOD_RES (7)..(7): Any amino acid
  MOD_RES (9)..(9): Any amino acid
  MOD_RES (12)..(15): Any amino acid
  MOD_RES (17)..(18): Any amino acid
  MOD_RES (20)..(26): Any amino acid
  MOD_RES (29)..(30): Any amino acid
ESXXVDXPXGNXXXXTXXFXXXXXXXNLXXL

SEQ ID NO: 100 - protein, 45 aa, Artificial Sequence
  Description of Artificial Sequence Synthetic PYL1-6 Arabidopsis PYR/PYL polypeptide consensus sequence
  MOD_RES (2)..(9): Any amino acid
  MOD_RES (11)..(11): Any amino acid
  MOD_RES (13)..(19): Any amino acid
  MOD_RES (22)..(25): Any amino acid
  MOD_RES (27)..(31): Any amino acid
  MOD_RES (33)..(34): Any amino acid
  MOD_RES (36)..(37): Any amino acid
  MOD_RES (40)..(40): Any amino acid
  MOD_RES (42)..(44): Any amino acid
HXXXXXXXXCXSXXXXXXXAPXXXXWXXXXXFXXPXXYKXFXXXC

SEQ ID NO: 101 - protein, 50 aa, Artificial Sequence
  Description of Artificial Sequence Synthetic PYL1-6 Arabidopsis PYR/PYL polypeptide consensus sequence
  MOD_RES (3)..(4): Any amino acid
  MOD_RES (6)..(6): Any amino acid
  MOD_RES (8)..(8): Any amino acid
  MOD_RES (10)..(10): Any amino acid
  MOD_RES (16)..(17): Any amino acid
  MOD_RES (19)..(19): Any amino acid
  MOD_RES (21)..(21): Any amino acid
  MOD_RES (23)..(25): Any amino acid
  MOD_RES (27)..(33): Any amino acid
  MOD_RES (35)..(37): Any amino acid
  MOD_RES (40)..(40): Any amino acid
  MOD_RES (44)..(44): Any amino acid
  MOD_RES (47)..(47): Any amino acid
VGXXRXVXVXSGLPAXXSXEXLXXXDXXXXXXXFXXXGGXHRLXNYXSVT

SEQ ID NO: 102 - protein, 33 aa, Artificial Sequence
  Description of Artificial Sequence Synthetic PYL1-6 Arabidopsis PYR/PYL polypeptide consensus sequence
  MOD_RES (2)..(2): Any amino acid
  MOD_RES (6)..(6): Any amino acid
  MOD_RES (9)..(9): Any amino acid
  MOD_RES (11)..(11): Any amino acid
  MOD_RES (14)..(17): Any amino acid
  MOD_RES (19)..(20): Any amino acid
  MOD_RES (22)..(22): Any amino acid
  MOD_RES (24)..(28): Any amino acid
  MOD_RES (32)..(32): Any amino acid
VXESYXVDXPXGNXXXXTXXFXDXXXXXNLQXL

SEQ ID NO: 103 - protein, 50 aa, Artificial Sequence
  Description of Artificial Sequence Synthetic PYL7-10 Arabidopsis PYR/PYL polypeptide consensus sequence
  MOD_RES (2)..(2): Any amino acid
  MOD_RES (4)..(8): Any amino acid
  MOD_RES (11)..(11): Any amino acid
  MOD_RES (13)..(13): Any amino acid
  MOD_RES (17)..(17): Any amino acid
  MOD_RES (19)..(19): Any amino acid
  MOD_RES (22)..(22): Any amino acid
  MOD_RES (24)..(24): Any amino acid
  MOD_RES (28)..(28): Any amino acid
  MOD_RES (34)..(34): Any amino acid
  MOD_RES (42)..(42): Any amino acid
  MOD_RES (46)..(46): Any amino acid
  MOD_RES (48)..(48): Any amino acid
  MOD_RES (50)..(50): Any amino acid
HXHXXXXXQCXSXLVKXIXAPXHXVWSXVRRFDXPQKYKPFXSRCXVXGX

SEQ ID NO: 104 - protein, 61 aa, Artificial Sequence
  Description of Artificial Sequence Synthetic PYL7-10 Arabidopsis PYR/PYL polypeptide consensus sequence
  MOD_RES (2)..(2): Any amino acid
  MOD_RES (4)..(5): Any amino acid
  MOD_RES (9)..(10): Any amino acid
  MOD_RES (18)..(18): Any amino acid
  MOD_RES (22)..(22): Any amino acid
  MOD_RES (25)..(25): Any amino acid
  MOD_RES (29)..(29): Any amino acid
  MOD_RES (34)..(34): Any amino acid
  MOD_RES (36)..(36): Any amino acid
  MOD_RES (38)..(38): Any amino acid
  MOD_RES (50)..(53): Any amino acid
  MOD_RES (55)..(55): Any amino acid
  MOD_RES (57)..(57): Any amino acid
  MOD_RES (59)..(59): Any amino acid
  MOD_RES (61)..(61): Any amino acid
EXGXXREVXXKSGLPATXSTEXLEXLDDXEHILXIXIXGGDHRLKNYSSXXXXHXEXIXGX

SEQ ID NO: 105 - protein, 44 aa, Artificial Sequence
  Description of Artificial Sequence Synthetic PYL7-10 Arabidopsis PYR/PYL polypeptide consensus sequence
  MOD_RES (1)..(1): Any amino acid
  MOD_RES (4)..(6): Any amino acid
  MOD_RES (15)..(15): Any amino acid
  MOD_RES (20)..(21): Any amino acid
  MOD_RES (24)..(24): Any amino acid
  MOD_RES (28)..(28): Any amino acid
  MOD_RES (31)..(31): Any amino acid
  MOD_RES (35)..(35): Any amino acid
  MOD_RES (39)..(41): Any amino acid
XGTXXXESFVVDVPXGNTKXXTCXFVEXLIXCNLXSLAXXXERL

SEQ ID NO: 106 - protein, 44 aa, Artificial Sequence
  Description of Artificial Sequence Synthetic PYL11-13 Arabidopsis PYR/PYL polypeptide consensus sequence
  MOD_RES (2)..(2): Any amino acid
  MOD_RES (4)..(5): Any amino acid
  MOD_RES (7)..(7): Any amino acid
  MOD_RES (10)..(10): Any amino acid
  MOD_RES (14)..(14): Any amino acid
  MOD_RES (22)..(22): Any amino acid
  MOD_RES (25)..(25): Any amino acid
  MOD_RES (27)..(31): Any amino acid
  MOD_RES (35)..(35): Any amino acid
  MOD_RES (37)..(39): Any amino acid
  MOD_RES (42)..(42): Any amino acid
CXSXXVXTIXAPLXLVWSILRXFDXPXXXXXFVKXCXXXSGXGG

SEQ ID NO: 107 - protein, 49 aa, Artificial Sequence
  Description of Artificial Sequence Synthetic PYL11-13 Arabidopsis PYR/PYL polypeptide consensus sequence
  MOD_RES (5)..(5): Any amino acid
  MOD_RES (8)..(8): Any amino acid
  MOD_RES (11)..(12): Any amino acid
  MOD_RES (15)..(15): Any amino acid
  MOD_RES (18)..(18): Any amino acid
  MOD_RES (22)..(22): Any amino acid
  MOD_RES (32)..(33): Any amino acid
  MOD_RES (39)..(39): Any amino acid
  MOD_RES (46)..(46): Any amino acid
GSVRXVTXVSXXPAXFSXERLXELDDESHVMXXSIIGGXHRLVNYXSKT

SEQ ID NO: 108 - protein, 40 aa, Artificial Sequence
  Description of Artificial Sequence Synthetic PYL11-13 Arabidopsis PYR/PYL polypeptide consensus sequence
  MOD_RES (17)..(18): Any amino acid
  MOD_RES (21)..(21): Any amino acid
  MOD_RES (23)..(24): Any amino acid
  MOD_RES (26)..(26): Any amino acid
  MOD_RES (28)..(28): Any amino acid
  MOD_RES (30)..(32): Any amino acid
  MOD_RES (35)..(35): Any amino acid
KKTVVVESYVVDVPEGXXEEXTXXFXDXIXXXNLXSLAKL

(19) United States
(12) Patent Application Publication
(10) Pub. No.: US 2022/0211037 A1
(43) Pub. Date: Jul. 7, 2022

(54) BIOSENSORS FOR DROUGHT STRESS IN PLANTS

(71) Applicant: The Regents of the University of California, Oakland, CA (US)

(72) Inventors: Ting GUO, Riverside, CA (US); Siobhan BRADY, Riverside, CA (US); Arjun SHARMAH, Riverside, CA (US); Sean CUTLER, Riverside, CA (US); Justin B. SIEGEL, Riverside, CA (US)

(21) Appl. No.: 17/609,227

(22) PCT Filed: May 6, 2020
(86) PCT No.: PCT/US20/31711

§ 371 Date: Nov. 5, 2021

Related U.S. Application Data
(60) Provisional application No. 62/844,479, filed on May 7, 2019.

(51) Int. Cl. A01N 37/36; C07K 14/415; C12N 15/82

(52) U.S. Cl. CPC A01N 37/36; C12N 15/8293; C07K 14/415

(57) ABSTRACT

Protein dimers that are modified to detect plant hormones are provided. In some embodiments, a protein dimer is provided comprising a first amino acid sequence and a second amino acid sequence, wherein the protein dimer dissociates in the presence of a plant hormone and the dissociation results in a detectable signal.

### CROSS-REFERENCES TO RELATED APPLICATIONS

This application claims the benefit of U.S. Provisional Application No. 62/844,479, filed May 7, 2019, which is incorporated by reference in its entirety herein for all purposes

### SEQUENCE LISTING

The instant application contains a Sequence Listing which has been submitted electronically in ASCII format and is hereby incorporated by reference in its entirety. Said ASCII copy, created on Mar. 11, 2020, is named 081906-1177328-231510PC_SL.txt and is 200,073 bytes in size.

### BACKGROUND OF THE INVENTION

In response to environmental stresses, plants can adjust growth and development using phytohormones. Facing drought or other stresses, for example, plants synthesize and respond to a terpenoid hormone called abscisic acid (ABA), which is involved in seed germination, seedling growth, regulation of stomatal aperture, flowering, and response to pathogens. (Cutler et al., Abscisic Acid: Emergence of a Core Signaling Network. Annu Rev Plant Biol 2010, 61, 651-679) The ABA signaling network in plants involves a class of water-soluble plant receptors called PYR/PYL/RCAR proteins, which form dimers in absence of ABA. Upon ABA-binding, the dimers for a subset of these proteins dissociate to their ABA-bound monomeric forms, which then regulate PP2C phosphatases and activate downstream SnRK2 kinases to activate many pathways including the control of stomata aperture. (Ma et al., Regulators of PP2C Phosphatase Activity Function as Abscisic Acid Sensors. Science 2009, 324 (5930), 1064-1068; Park et al., Abscisic Acid Inhibits Type 2C Protein Phosphatases via the PYR/PYL Family of START Proteins. Science 2009, 324 (5930), 1068-1071)) In the absence of ABA, the dimeric receptors are autoinhibited, enabling PP2C phosphatases to bind to SnRK2 kinases and render them inactive.

Throughout evolution, this response to drought stress has been finely tuned, making it challenging for humans to detect subtle, yet physiologically relevant changes in ABA concentration without the use of transgenic reporters. Thus, an engineered interface to in vivo ABA signaling utilizing endogenous components as biosensors could enable real time human-mediated mitigation of drought. These sensors would allow plants to report to farmers or automated irrigation systems to obtain point-of-mitigation. To meet all these requirements, the sensors need to be based on endogenous biomolecules and respond to drought stress. Since the PYL proteins comply with these requirements, they present viable candidates to engineer a biosensor.

PYL-based sensors have been investigated by designing recombinant proteins that incorporate fluorescent domains to enable optical sensing using principles such as Förster resonance energy transfer (FRET). One report demonstrated the quantification of micromolar (μM) concentrations of ABA in plants by obtaining the ratio of fluorescence intensities in two spectral regions from two fluorophores, one attached to a PYL protein and the other to a phosphatase. (Waadt et al., FRET-based reporters for the direct visualization of abscisic acid concentration changes and distribution in Arabidopsis. Elife 2014, 3) Upon ABA binding, the phosphatase binds to the PYL to enable FRET, causing decrease of fluorescence in one spectral region and increase in the other. Other similar methods have been developed. (Jones et al., Abscisic acid dynamics in roots detected with genetically encoded FRET sensors. Elife 2014, 3) These pioneering works suggest that it is possible to develop sensitive optical sensors to detect ABA in plants.

### BRIEF SUMMARY OF THE INVENTION

In some embodiments, a protein dimer is provided comprising a first amino acid sequence and a second amino acid sequence, wherein the protein dimer dissociates in the presence of a plant hormone and the dissociation results in a detectable signal. In some embodiments, the plant hormone is abscisic acid (ABA). In some embodiments, the dimer is a heterodimer. In some embodiments, the dimer is a homodimer.

In some embodiments, one or more of the first and second amino acid sequences is a PYL protein. In some embodiments, the PYL protein is not covalently linked to a phosphatase. In some embodiments, the PYL protein is a PYL3 protein.

In some embodiments, the first amino acid sequence comprises a fluorescent protein sequence and the second amino acid molecule comprises a first quencher protein sequence. In some embodiments, the first amino acid sequence is conjugated to a first dye molecule and the second amino acid molecule is conjugated to a first quencher. In some embodiments, the first quencher is also a dye molecule that emits a detectable signal. In some embodiments, the first dye molecule is also a quencher with respect to the detectable signal of the first quencher. In some embodiments, the dye molecules are self-quenching such that when two of the dye molecules are in proximity (as part of the dimer) their signal is quenched compared to when not in proximity (when in monomeric form). In some embodiments, the detectable signal is florescent or colorometric. In some embodiments, the dye molecule is a fluorophore.

Also provided is a plant comprising one or more exogenous genes encoding the first and second amino acid sequences as described above or elsewhere herein. In some embodiments, a plant is provided expressing the first and second amino acid sequences.

Also provided is a method of monitoring plant hormones in a plurality of adjacent plants. In some embodiments, at least one plant in the plurality is the plant as described above or is a plant comprising the first amino acid sequence and the second amino acid sequence. In some embodiments, the method comprises detecting the detectable signal from the at least one plant in the plurality. In some embodiments, the first amino acid sequence and the second amino acid sequence are injected (or otherwise introduced into the plant non-transgenically) into the plant. In some embodiments, the first and second amino acid sequences are identical and the amino acid sequences are linked to a self-quenching fluorescent label.

In some embodiments, the method further comprises altering at least one environmental condition of the plurality if the level of detectable signal exceeds or is below a threshold value In some embodiments, the altering comprises providing the plurality water or nutrients or pesticides.

In some embodiments, the detecting is performed by a detector over the plurality of plants. In some embodiments, the detector is a rover or an aerial drone.

### BRIEF DESCRIPTION OF THE DRAWINGS

FIG. 1 is a graph showing mass to charge ratios of PYL3 (blue) and the conjugates of Cy5.5 (red) and BHQ3 (black). Reaction yields were approximately 20%, even though MALDI detects only PYL3 dye conjugates when mixed with PYL3.

FIG. 2A is a graph of UV-Vis absorption measurements of PYL3 (blue line), PYL3-Cy5.5 (red line) and PYL3-BHQ3 (black line). The data is used to estimate conjugation reaction yield.

FIG. 2B is a graph of fluorescence measurement of PYL3-Cy5.5 and PYL3-BHQ3. The latter yields little fluorescence.

FIG. 2C is a graph of fluorescence of PYL3-Cy5.5 as a function of varying ABA concentrations, demonstrating self-quenching.

FIG. 2D presents a graph of the average molar mass of the three species measured with SLS, which shows that ABA dissociates PYL3 dimers. Green=ABA ligand. Also presented is a table of calculated amounts of PYL3-Cy5.5 in dimer and monomer forms (in percentage) at 0.1, 1, 10, 100 and 1000 μM ABA. Even at 1 mM ABA, 24% of PYL3-Cy5.5 exists in the dimer form.

FIG. 3A is a graph of UV-Vis absorption profiles for PYL3-Cy5.5 and PYL3-BHQ3 mixtures.

FIG. 3B is a graph of fluorescence profiles for PYL3-Cy5.5 and PYL3-BHQ3 mixtures.

FIG. 3C presents graphs of the fluorescence of free BHQ3/Cy5.5 as a function of the monomer or dimer concentrations. Attenuation is observed when Cy5.5 is mixed with BHQ3 (linear up to nine μM for both components).

FIG. 3D is a graph of the absorption spectra of Cy5.5 and BHQ3.

FIG. 3E is a graph of the fluorescence response of the mixture as a function of ABA concentration (red line) and SLS measurements as a function of ABA concentration (black line). The two both show transitions between 10 and 100 μM of ABA.

FIG. 4A is a graph showing experimental SLS (dashed line) and simulated results of average molar mass from simulations to predict the equilibrium concentrations of monomers and homodimers based on association/disassociation equations and binding constants. The k values used in the simulation are: k₁=1.5-5.0, k₋₁=0.6-2.0, k₂=1.5-6.0, k₋₂=0.6-2.0, k₃=0.6-2.0, k₋₃=1.3-6.0, k₄=5.0-15.0, and k₋₄=7.2×10⁻⁶-5.2×10⁻⁵. The simulated total fluorescence (f) is calculated as: and f=I₀×(R_{[ABA]}/R_{No ABA}), where I₀=3750 cps, which is the initial fluorescence signal of the heterodimer mixture measured prior to ABA addition. R_{[ABA]} is the calculated fluorescence signal factors of heterodimer mixture as a function of added ABA, and R_{No ABA} is the factor without ABA. The R factors and the concentrations of different species obey the following relationship: R=([PF-ABA]+0.2×[PFPQ]+0.8×[PFPF]+[PF]) where [PF-ABA] and [PF] are the concentration of PYL3-Cy5.5-ABA and PYL3-Cy5.5, and [PFPQ] and [PFPF] are the concentration of PYL3-Cy5.5/PYL3-BHQ3 homodimers and PYL3-Cy5.5/PYL3-Cy5.5 homodimers.

FIG. 4B is a graph showing experimental (dashed line) and theoretical (solid line and the shaded trace) simulation results of the total fluorescence signals as a function of ABA concentrations.

FIG. 5A is a graph of a free phosphate standard calibration curve using molybdate dye obtained after background subtraction. In the linear fit equation y is the absorbance at 630 nm value whereas x is the concentration of free phosphate. The free 1 mM phosphate standard (KH₂PO₄) is supplied by Promega Phosphatase Assay System.

FIG. 5B is a graph showing the relative phosphate activity (%) as a function of ABA concentration.

FIG. 6 illustrates a sensing design to detect ABA molecules. The top panel displays the FRET-based quenching between a fluorophore Cy5.5 in PYL3-Cy5.5 and a quencher BHQ3 in PYL3-BHQ3. FRET quenching is shown as the dimmed Cy5.5 in the heterodimer. The middle panel describes the sensor preparation including Cy5.5-PYL3 and BHQ3-PYL3 monomers and homodimers. Self-quenching is represented by the slightly dimmed Cy5.5 in PYL3-Cy5.5 homodimers. Legends are explained in the lower panel.

FIG. 7A is a graph of UV-Vis absorbance as a function of Cy5.5-NHSEster concentration.

FIG. 7B is a graph of UV-Vis absorbance as a function of BHQ3-NHSEster concentration.

FIG. 8 is a graph of the change in molar mass of PYL3, PYL3-Cy5.5, and PYL3-BHQ3 homodimers as a function of increasing ABA content performed with lower protein/protein-dye conjugate concentration of 1.9 μM. The solid line is the theoretical simulation with k values of k₁=0.8-1.1, k₋₁=0.05-0.08, k₂=0.8-1.1, k₋₂=0.05-0.08, k₃=0.05-0.08, k₋₃=0.8-1.1, k₄=6.0×10⁻⁶-7.2×10⁻⁶, k₋₄=6.3-6.5. Decrease in molar masses as a function of added ABA indicates that the initial homodimers are increasingly converted to monomers as concentration of ABA in the system increases

### DEFINITIONS

The term “PYR/PYL receptor polypeptide” refers to a protein characterized in part by the presence of a polyketide cyclase domain, for example as identified by PFAM domains: polyketide cyclase domain 2 (PF10604) or polyketide cyclase domain 1 (PF03364), which in wild-type form mediates abscisic acid (ABA) and ABA analog signaling. A wide variety of PYR/PYL receptor polypeptide sequences are known in the art. In some embodiments, a PYR/PYL receptor polypeptide comprises a polypeptide that is substantially identical to Arabidopsis PYR1 (SEQ ID NO:1), PYL1 (SEQ ID NO:2), PYL2 (SEQ ID NO:3), PYL3 (SEQ ID NO:4), PYL4 (SEQ ID NO:5), PYL5 (SEQ ID NO:6), PYL6 (SEQ ID NO:7), PYL7 (SEQ ID NO:8), PYL8 (SEQ ID NO:9), PYL9 (SEQ ID NO:10), PYL10 (SEQ ID NO:11), PYL11 (SEQ ID NO:12), PYL12 (SEQ ID NO:13), or PYL13 (SEQ ID NO:14), or to any of SEQ ID NOS:15-89.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 60% sequence identity with a reference sequence. Alternatively, percent identity can be any integer from 60% to 100%. Some embodiments include at least: 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below. Embodiments of the present invention provide for nucleic acids encoding polypeptides that are substantially identical to any of SEQ ID NOS:1-89 and have at least one of the amino acid mutations described herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

It is contemplated that a substitution mutation in a mutated PYR/PYL receptor polypeptide includes amino acids that are conservative substitutions for those specific amino acids, so long as the conservatively substituted amino acid is not the wild-type amino acid. As a non-limiting example, where a mutated PYR/PYL receptor polypeptide comprises a serine-to-threonine substitution, it is contemplated that the mutated PYR/PYL receptor polypeptide may alternatively comprise a serine-to-alanine substitution, as threonine and alanine are conservative substitutions for one another; but the mutated PYR/PYL receptor polypeptide would not comprise a serine-to-serine substitution, as serine is the amino acid that is present in the wild-type PYR/PYL polypeptide.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence.

### DETAILED DESCRIPTION OF THE INVENTION

### Introduction

The inventors have discovered a new way to monitor plant hormone levels. Protein dimers that form in the presence but not in the absence of, or alternatively in the absence but not the presence of, a plant hormone can be engineered to generate a detectable signal (optionally a change in signal) based on the binding of the plant hormone to the protein dimer. The quantity of detectable signal can therefore be used to measure the amount of plant hormone in a plant. This can in turn be used to optimize plant growth and health by adjusting one or more environmental factors (e.g., water, nutrients, pesticides, etc.) based on the detected level of plant hormone in the plant. Such plants carrying the detection system described herein can be included in a plurality of other plants (e.g., as a row within a field of non-detector plants) to indicate the level of plant hormone in the plurality of plants).

### Protein Dimers

Any protein that dimerizes upon binding a plant hormone, or alternatively, that monomerizes upon binding to a plant hormone can be used. The protein dimer can be a heterodimer or a homodimer. In either case, the dimer is made up of two separate proteins. As described herein, in some embodiments, a first protein of the dimer can be linked (e.g., either chemically conjugated otherwise covalently linked) to a first signal generating molecule and the second protein of the dimer can be linked to a second molecule that is capable of altering the signal from the first signal generating molecule when in proximity (e.g., when the dimer is formed) compared to when the not in proximity (when the proteins are in monomeric form). Thus, a change in signal is generated when the plant hormone binds compared to when it is not bound. In some embodiments, the first signal generating molecule and the second signal generating molecule are identical. Alternatively, in some embodiments, the first signal generating molecule and the second signal generating molecule are different. Examples of different signal generating molecules include, e.g., embodiments in which one generates a signal and the other quenches that signal when in proximity or embodiments in which signal is generated when the two signal generating molecules are in proximity but not when they are not in proximity.

A non-limiting example of a protein that binds a plant hormone is a protein from the PYR/PYL protein family, which are receptors for abscisic acid. A wide variety of wild-type (naturally occurring) PYR/PYL polypeptide sequences are known in the art. Although PYR1 was originally identified as an abscisic acid (ABA) receptor in Arabidopsis, in fact PYR1 is a member of a group of at least 14 proteins (PYR/PYL proteins) in the same protein family in Arabidopsis that also mediate ABA signaling. This protein family is also present in other plants (see, e.g., SEQUENCE LISTING) and is characterized in part by the presence of a polyketide cyclase domain, for example as identified by PFAM domains: polyketide cyclase domain 2 (PF10604) or polyketide cyclase domain 1 (PF03364). See, e.g., Finn et al., Nuc. Acids Res. 42:D222-230 (2013) describing PFAM domains. These sorts of domains are part of the START/Bet v 1 superfamily domain, which are described in, for example, Radauer, BMC Evol. Biol. 8:286 (2008). Klinger et al. J. Exp. Botany 61(12):3199-3210 (2010); Melcher et al. Nature 462:602-610 (2009); and Santiago et al., Nature 462:665-669 (2009) each describe structural features of the PYR/PYL protein family. In some embodiments, a wild-type PYR/PYL receptor polypeptide comprises any of SEQ ID NOs:1-89. In some embodiments, a wild-type PYR/PYL receptor polypeptide is substantially identical to (e.g., at least 70%, 75%, 80%, 85%, 90%, 91%, 92%, 93%, 94% 95%, 96%, 97%, 98%, or 99% identical to) any of SEQ ID NOs:1-89.

In situations where additional variants or orthologs of the above sequences are desired, it can be useful to generate sequence alignments to identify conserved amino acid or motifs (i.e., where alteration in sequences may alter protein function) and regions where variation occurs in alignment of sequences (i.e., where variation of sequence is not likely to significantly affect protein activity). Some useful consensus sequences for identifying PYR/PYL polypeptides include, e.g., EXLXXXD GGXHXL (SEQ ID NO:90), CxSxxxxxxxAPxxxxWxxxxxFxxPxxxxxFxxxC (SEQ ID NO:91), GxxRxVxxxSxxPAxxSxExLxxxD (SEQ ID NO:92), and/or GGxHRLxNYxS (SEQ ID NO:93). In addition, more specific consensus sequences can be represented by aligning subsets of the 14 members of the Arabidopsis PYR/PYL proteins, though these consensus sequences are more broadly applicable to other plant orthologous sequences. Examples of such consensus sequences include, e.g.,

PYR1 to PYL12
(SEQ ID NO: 94)
CxSxxxxxxxAPxxxxWxxxxxFxxPxxxxxFxxxC
(SEQ ID NO: 95)
GxxRxVxxxSxxPAxxSxExLxxxD
(SEQ ID NO: 93)
GGxHRLxNYxS
(SEQ ID NO: 96)
ESxxVDxPxGxxxxxTxxFxxxxxxxNLxxL
PYL1-12 consensus
(SEQ ID NO: 97)
CxSxxxxxxxAPxxxxWxxxxxFxxPxxxKxFxxxC
(SEQ ID NO: 98)
GxxRxVxxxSxLPAxxSxExLxxxD
(SEQ ID NO: 93)
GGxHRLxNYxS
(SEQ ID NO: 99)
ESxxVDxPxGNxxxxTxxFxxxxxxxNLxxL
PYL1-6 Consensus
(SEQ ID NO: 100)
HxxxxxxxxCxSxxxxxxxAPxxxxWxxxxxFxxPxxYKxFxxxC
(SEQ ID NO: 101)
VGxxRxVxVxSGLPAxxSxExLxxxDxxxxxxxFxxxGGxHRLxNYxSV
T
(SEQ ID NO: 102)
VxESYxVDxPxGNxxxxTxxFxDxxxxxNLQxL
PYL7-10 Consensus
(SEQ ID NO: 103)
HxHxxxxxQCxSxLVKxIxAPxHxVWSxVRRFDxPQKYKPFxSRCxVxG
x
(SEQ ID NO: 104)
ExGxxREVxxKSGLPATxSTExLExLDDxEHILxIxIxGGDHRLKNYSS
xxxxHxExIxGx
(SEQ ID NO: 105)
xGTxxxESFVVDVPxGNTKxxTCxFVExLIxCNLxSLAxxxERL
PYL11-13 Consensus
(SEQ ID NO: 106)
CxSxxVxTIxAPLxLVWSILRxFDxPxxxxxFVKxCxxxSGxGG
(SEQ ID NO: 107)
GSVRxVTxVSxxPAxFSxERLxELDDESHVMxxSIIGGxHRLVNYxSKT
(SEQ ID NO: 108)
KKTVVVESYVVDVPEGxxEExTxxFxDxIxxxNLxSLAKL.

Accordingly, in some embodiments, the PYR/PYL polypeptides as described herein comprise one or more of the above-described consensus sequences or conservative variants thereof.

Other plant dimers that bind to plant hormones (in dimeric or in monomeic form) can also be used. Other examples include, but are not limited to, TIR1/AUXIAA (see, e.g., Dharmasiri, et al., Nature, volume 435, pages 441-445(2005)); GID1/DELLA (see, e.g., Uns, Plant Physiology, October 2010, Vol. 154, pp. 567-570); JAZ/COI (see, e.g., Chini et al., The FEBS Journal, Volume 276, Issue 17, September 2009, Pages 4682-4692).

In some embodiments, the protein dimer dissociates in the presence of a plant hormone (e.g., such as ABA) and the dissociation results in a detectable signal. For example, the PYR/PYL proteins form dimers in the absence of ABA and form monomers when binding ABA.

Detectable signal can be generated in a number of ways. For example, by attaching a signal generating molecule to each monomer wherein the signal of the signal generating molecule(s) change when in proximity (when in a dimer) compared to being separate (e.g., monomeric), the presence and quantity of ABA can be determined. An example of molecules that change signal generation depending on their proximity are molecules that use fluorescence resonance energy transfer (FRET) technology. For example, one signal generating molecule can be a reporter (e.g., a fluorescent reporter) and the other signal generating molecule can be a quencher of the reporter. In such a case, the dimerized protein will not fluoresce or will fluoresce at a measurably lower level due to quenching compared to when the protein binds the plant hormone and is in the monomeric form. Suitable reporters and quenchers include, for example, black hole quencher dyes (BHQ), TAMRA, FAM, CY3, CYS, Fluorescein, HEX, JOE, LightCycler Red, Oregon Green, Rhodamine, Rhodamine Green, Rhodamine Red, ROX, TAMRA, TET, Texas Red, and Molecular Beacons.

Alternatively, the signal generating molecules can be protein sequences. In such embodiments, the monomers can be encoded in the genome of a plant and can be expressed by the plant. For example, the plant hormone-binding protein can be expressed in two forms: first, as a fusion with a fluorescent protein and second as a fusion with a protein that quenches the fluorescent protein when in proximity. This will be particularly effective in the case where the fluorescent label is self-quenching such that the dimer, when formed, quenches signal compared to monomeric form. Examples of protein sequences that can function as FRET pairs include but are not limited to those described in Bajer, et al., Sensors (Basel) September; 16(9): 1488 (2016) and George Abraham B, et al. PLoS ONE 10(8): e0134436 (2015). In this way, the two protein forms will form dimers in the absence of hormone and will monomerize in the presence of the hormone, thereby resulting in an increase in signal that can be subsequently detected.

As mentioned above, the signal generating molecule (which can be a protein sequence) can be self-quenching such that a homo-dimer of the protein results in quenching of the fluorescent signal of the signal generating molecules when in monomeric form, but wherein signal is generated when the proteins are dimerized. As an example, Cy5.5 is self-quenching though other self-quenching molecules can be selected depending on precise requirements of an assay.

Signal from the signal generating molecules can be detected as appropriate for the type of signal emitted. For example, any type of photon detection or other detector can be used. In some embodiments, the signal generating molecules are initially excited at a certain wavelength (e.g., by a laser) and the resulting light emitted is detected.

In some embodiments, signal is detected by a self-guided or human-guided vehicle, including for example a flying vehicle such as a satellite, airplane, drone or rover. Such vehicles can allow for detection across a large area, such as a farm or field. See, for example, European patent publication EP 1125111A1.

In some embodiments, the protein(s) are introduced into the plant by non-transgenic (non-transgenic plant) methods. For example the proteins can be introduced by injection of the proteins or of a vector encoding the proteins where the vector is not integrated into the genome of the plant.

As discussed herein, in some embodiments plants are generated to express proteins that form dimers, wherein the dimer proteins are fusions with fluorescent or quenching polypeptide sequences. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a PYR/PYL or other coding sequence driven by a heterologous promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of the constitutively active PYR/PYL receptor is encompassed by the invention, generally expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983). In some embodiments, the Agrobacterium is introduced via infiltration.

Transformed plant cells derived by any of the above transformation techniques can be cultured to regenerate a whole plant that possesses the transformed genotype and thus the desired phenotype such as enhanced abiotic stress resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes can be used to confer abiotic stress resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. In some embodiments, the plant is selected from the group consisting of rice, maize, wheat, soybeans, cotton, canola, turfgrass, and alfalfa. In some embodiments, the plant is an ornamental plant. In some embodiment, the plant is a vegetable- or fruit-producing plant.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

### Examples

Abscisic acid (ABA) is an essential drought stress molecule and simple methods for detecting its levels could benefit agriculture. Here, we present as proof-of-concept a biosensor to detect ABA in aqueous solutions using a Cyanine5.5 (Cy5.5) fluorophore- and BHQ3 quencher conjugated to endogenous abscisic acid receptor pyrabactin resistance 1 like proteins (PYL3), which monomerize upon ABA binding. A mixture of the two protein conjugates was used to detect nM to mM ABA in aqueous solution. As the ABA concentration increased from less than one μM to one mM, fluorescence intensity of the mixture more than doubled. In addition to BHQ3 quenching the fluorescence of Cy5.5 in PYL3-Cy5.5/PYL3-BHQ3 heterodimers, self-quenching was observed between two fluorophores in PYL3-Cy5.5 homodimers. BHQ3 concentration-dependent attenuation of Cy5.5 was observed as well, which was significant at the BHQ3 concentrations used in this work. A kinetic model was developed to simulate the fluorescence response from the mixture and the results generally agree with the experimentally observed trend. This work demonstrates that fluorescence measurements of a single dissociation reaction in one spectral region are adequate to assess the ABA concentration of a solution.

To simplify ABA detection so that detection in only a single spectral region is possible, we developed a new sensing mechanism using components of the ABA signaling pathway. This design can potentially provide more possibilities for development of sentinel plants to report drought stress by conjugating small molecules to PYL proteins. The principle behind this work is based on FRET quenching between a fluorophore and a quencher chemically linked to PYLs. In this process, the quencher resonantly absorbs and dissipates the energy released from the excited fluorophore and reduces or eliminates fluorescence from the fluorophore. For efficient quenching through FRET, the fluorophore and quencher need to be located within a few nanometers of each other. (Meer, B. W. v. d., FRET—Forester Resonance Energy Transfer: From Theory to Applications. Wiley-VCH/Verlag GmbH & Co. KGaA: Weinheim, Germany, 2014) Our design takes advantage of the dimerization of the PYL receptors to establish this close proximity. The Arabidopsis thaliana genome encodes 14 PYL proteins, each with a different monomer—dimer dissociation equilibrium. (Hao et al., The Molecular Basis of ABA-Independent Inhibition of PP2Cs by a Subclass of PYL Proteins. Mol Cell 2011, 42 (5), 662-672) Without ABA, a significant portion of the PYL receptors stay in the dimeric form. In the presence of ABA the equilibrium shifts towards PYL monomers which facilitates binding of the protein phosphatase 2C ABSCISIC ACID INSENSITIVE 1 (ABI1). For instance, the homodimeric receptor PYL3 has an equilibrium dissociation constant (K_{d}) of 7.76 μM, which increases to 52 μM when at saturating ABA concentrations. (Zhang et al., Complex Structures of the Abscisic Acid Receptor PYL3/RCAR13 Reveal a Unique Regulatory Mechanism. Structure 2012, 20 (5), 780-790) This increase in the dissociation constant is attributed to a conformational change to the dimers from the initial cis-homodimer to the trans-homodimer upon ABA binding. (Zhang et al., Complex Structures of the Abscisic Acid Receptor PYL3/RCAR13 Reveal a Unique Regulatory Mechanism. Structure 2012, 20 (5), 780-790; Zhang et al., Structural basis and functions of abscisic acid receptors PYLs. Front Plant Sci 2015, 6) Therefore, if a fluorophore is conjugated to one PYL3 monomer and a quencher to the corresponding monomer, then the heterodimer would switch from the quenched state to the fluorescent state upon ABA binding, which results in increased fluorescence in the presence of increased concentrations of ABA in drought stressed plants.

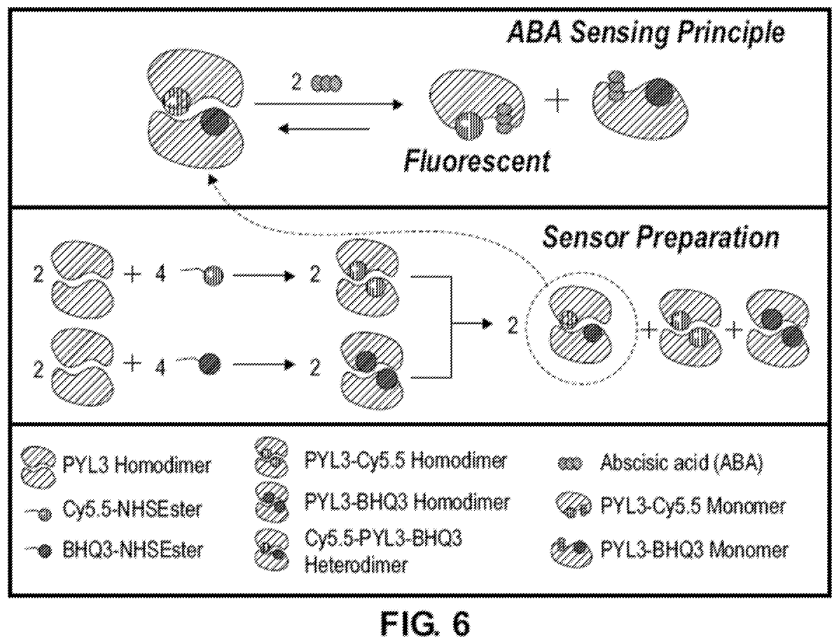

The design employed in this work is described in FIG. 6 (top panel). PYL3 was used because it presents the highest dimeric dissociation constant. (Hao et al., The Molecular Basis of ABA-Independent Inhibition of PP2Cs by a Subclass of PYL Proteins. Mol Cell 2011, 42 (5), 662-672) A fluorescent molecule, Cy5.5, is chemically conjugated to one protein monomer to form PYL3-Cy5.5. In a separate synthesis, a quencher, BHQ3, is chemically conjugated to another monomer of the same protein, forming PYL3-BHQ3 (mid panel). Equal amounts of PYL3-Cy5.5 and PYL3-BHQ3 monomers are mixed to ensure a maximum optical signal change upon ABA binding. By controlling the synthesis conditions of these small molecule-protein fusions, only one fluorophore or one quencher is conjugated to each PYL3 protein. Conjugation of fluorophores and quenchers are not anticipated to influence the dimerization of PYL3, their binding of the ABA ligand, or the binding to other biomolecules such as the protein phosphatase 2C. In vitro tests of the sensing mechanism described in FIG. 6 demonstrate that the sensor detects ABA through its interactions with the modified PYL3 proteins in water. The fluorescent signal intensifies with increasing ABA concentrations. With these encouraging results it is anticipated that this sensing mechanism can be utilized to design biomolecule-based endogenous drought sensors to report drought stress when interfaced with external optical excitation and detection devices.

### Materials

pET28 and BL21[DE3] E. coli were used. (Cutler et al., A. Control of plant stress tolerance, water use efficiency and gene expression using novel ABA receptor proteins and synthetic agonists. 2016) Growth media for E. coli (pET28) including LB Agar, tryptone, and yeast extract were purchased from BD. Sodium chloride, HEPES, buffer, TRIS Buffer, triethanolamine, aluminum sulfate, sodium phosphate dibasic, sodium phosphate monobasic, Coomassie brilliant blue R, and polyacrylamide were purchased from Sigma-Aldrich. Kanamycin, chloramphenicol, isopropyl-β-D-thiogalactoside (IPTG), imidazole, and regenerated cellulose dialysis tubing were purchased from Thermo Fisher Scientific. A HisTrap HP column was purchased from GE Healthcare. A non-radioactive phosphatase assay system (Ser/Thr) was purchased from Promega. Cyanine5.5 NHS ester was purchased from Lumiprobe and BHQ3 NHS ester was purchased from Biosearch Technologies.

### Protein Expression, Purification, and Dye Conjugation

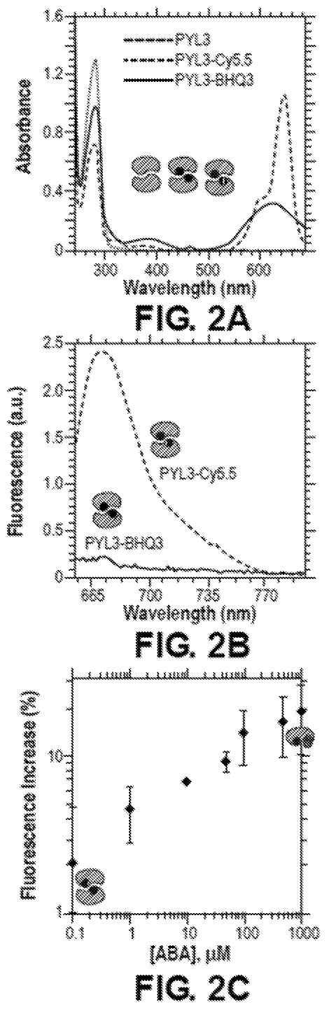

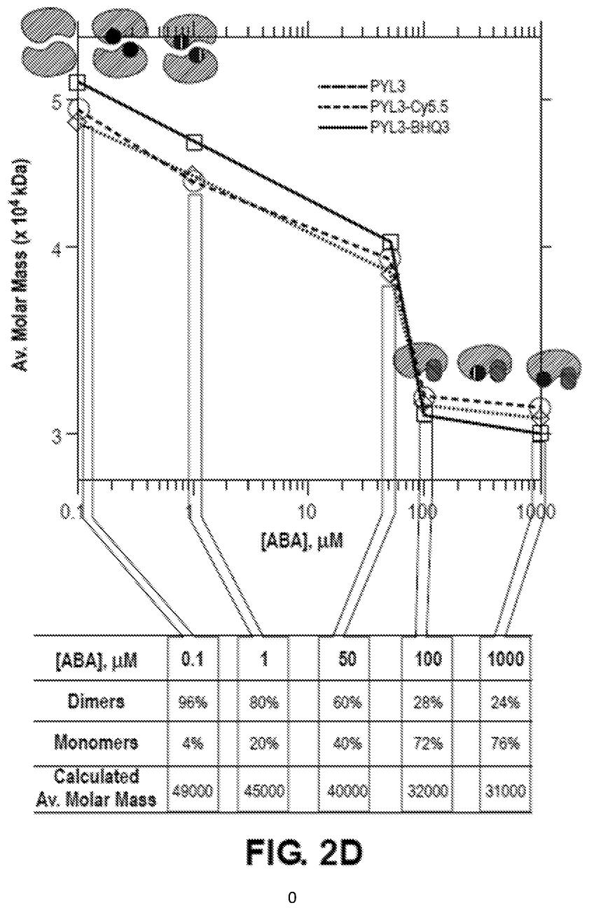

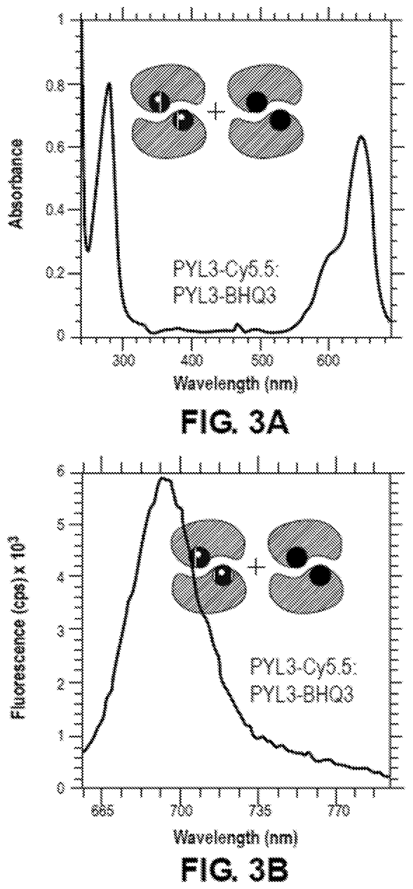

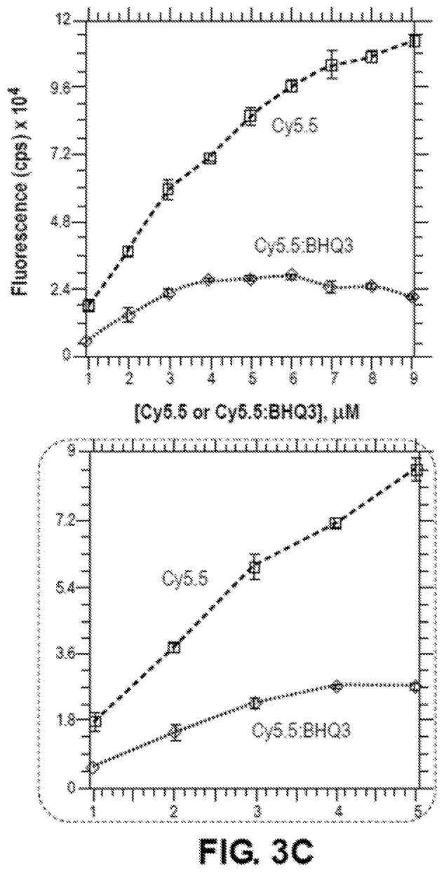

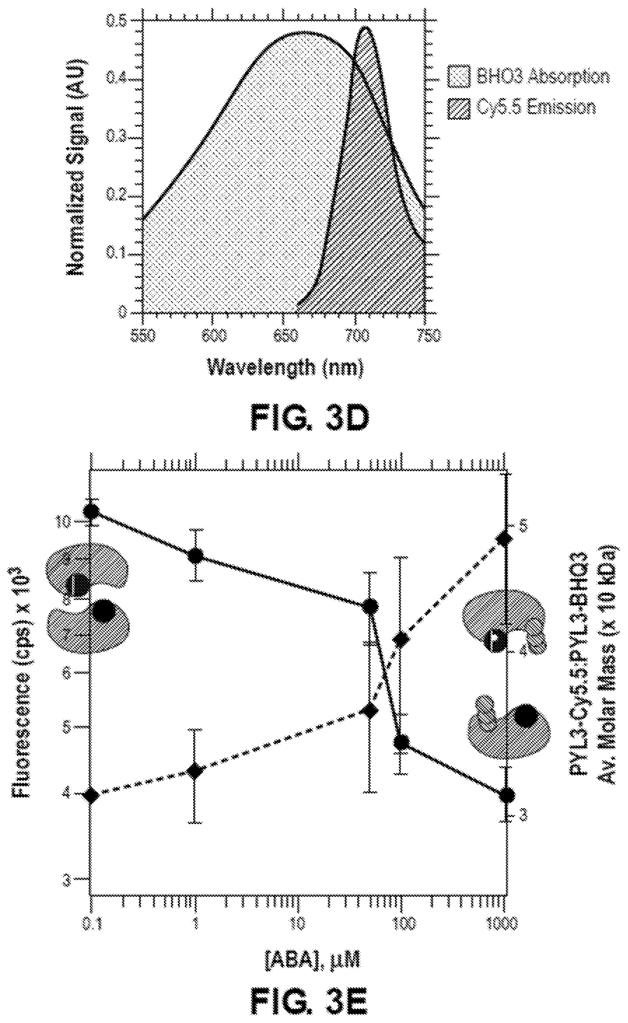

pET28 E. coli with the required plasmids were used for expression and to prepare glycerol stocks with a final glycerol content of 25% for long-term storage at −80° C. Protein expression was induced by cultivating E. coli in the presence of 0.1 mM IPTG. A pre-culture was grown over night in a 12-mL culture tube at 37° C. The main culture was started with an optical density of 0.05-0.1 from the preculture. IPTG induction (0.1 mM final concentration) occurred once the E. coli reached an optical density of 0.3-0.5 at a wavelength of 605 nm and proceeded up to 6 hours at 28° C. After terminating the expression on ice for approximately 5 min, the cells were centrifuged for 10 minutes at 4000 g. The pellet was washed using 5 mL of purification buffer (20 mM PB and 500 mM NaCl of pH 7.4). Protein extraction and purification were carried out at 4° C. The cells were suspended in 8 mL denaturation purification buffer (20 mM PB, 500 mM NaCl, 20 mM imidazole and 8 M urea) and the proteins were extracted following an established protocol. (Feliu et al., Optimized release of recombinant proteins by ultrasonication of E. coli cells. Biotechnol Bioeng 1998, 58 (5), 536-540) All media were autoclaved prior to use. The proteins were purified using a HisTrap HP 1 mL column, which was prepared by washing with 5 mL of 20% ethanol, 5 mL of MilliQ water, and 5 mL of binding buffer (20 mM PB, 500 mM NaCl at pH 7.4) at a flow rate of 1 mL/min. Proteins were applied at a flow rate of 0.15 mL/min followed by sequential rinsing with low imidazole concentrations present. 5 mL of binding buffer, 5 mL of wash buffer #1 (20 mM PB, 500 mM NaCl, 20 mM imidazole at pH 7.4), 5 mL of wash buffer #2 (20 mM PB, 500 mM NaCl, 40 mM imidazole at pH 7.4), and 5 mL of wash buffer #3 (20 mM PB, 500 mM NaCl, 60 mM imidazole at pH 7.4) were subsequently applied at 1 mL/min flow rate. Ten protein fractions were eluted using 10 mL elution buffer (20 mM PB, 500 mM NaCl, 500 mM imidazole at pH 7.4) at a flow rate of 1 mL/min. The column was restored by sequentially flushing with 5 mL of the elution buffer, of milli-Q water, and of 20% ethanol at 1 mL/min. The elution fractions were tested using SDS-PAGE gels. Fractions with similar amounts of the pure protein were combined and the molar mass was verified using MALDI-MS. The protein concentration was determined using UV-Vis absorption spectroscopy at 280 nm. The protein dimer has a molar extinction coefficient of 9250 M⁻¹ cm⁻¹. The absorption at 280 nm was background corrected using the UV-Vis absorption spectra (FIGS. 2A and 3A). Proteins were concentrated using reverse dialysis to obtain the final solutions. The purified homodimers were conjugated to Cy5.5 and BHQ3 NHS ester at an approximately 1:6 protein-to-dye ratio and a pH of 8.3. Prior to conjugation, 400 μL of 0.6 M sodium bicarbonate solution was added into 2 mL of approximately 1.9 mg/mL protein solution and the mixture was vortexed for less than a minute. Conjugation to Cy5.5 NHS ester and BHQ3 NHS ester was accomplished by adding 133 μL of 3.9 mM dye in DMSO to 1.2 mL of the prepared protein solution at 4° C. and the mixture was allowed to react for 12 hours. The products were purified by triple dialysis in 20 mM PB. Concentration of the dye conjugated proteins was determined using UV-Vis absorption spectroscopy. Reverse dialysis was used to obtain PYL3 proteins that contained Cy5.5 or BHQ3 conjugated proteins. Fisherbrand regenerated cellulose tubing with nominal MWCO of 12,000-14,000 was used for dialysis. The Cy5.5 conjugated homodimer protein and BHQ3 conjugated homodimer protein were mixed in equal volumes (1 mL each) and incubated for 24 hours at 4° C. to establish an exchange equilibrium with dye-conjugated heterodimers.

### Characterization Methods

For fluorescence or molar mass measurements of the proteins as a function of ABA concentration, 0.6 mL of the conjugated protein solutions containing approximately equal amounts of PYL3-Cy5.5 and PYL3-BHQ3 (20% of protein conjugates in the presence of PYL3) were mixed with 0.6 mL of the specified concentration of ABA (aq.) solution by vortexing and incubation for 30 min. UV-Vis absorption measurements were performed on PYL3-Cy5.5, PYL3-BHQ3, and a 1:1 mixture of PYL3 containing PYL3-Cy5.5 and PYL3-BHQ3 (Shimadzu UV-Vis-NIR spectrophotometer, UV1700). Fluorescence measurements were performed on PYL3-Cy5.5, PYL3-BHQ3, and 1:1 mixture of PYL3 containing PYL3-Cy5.5 and PYL3-BHQ3 and as a function of ABA concentration (Yvon-Horiba FluoroMax-4). Static light scattering (SLS) was used to determine the average molar mass (Malvern Zetasizer Nano S90 fitted with a 633 nm He—Ne laser). The mass of PYL3, PYL3-Cy5.5 and PYL3-BHQ3 was measured using Bruker UltraFlextreme MALDI TOF. Samples for MALDI-TOF measurement were prepared using a dried droplet method in a 2,5-Dihydroxyacetophenone (2,5-DHAP) matrix. The mass spectra were acquired with laser repetition frequency of 0.7-1 kHz.

### Results

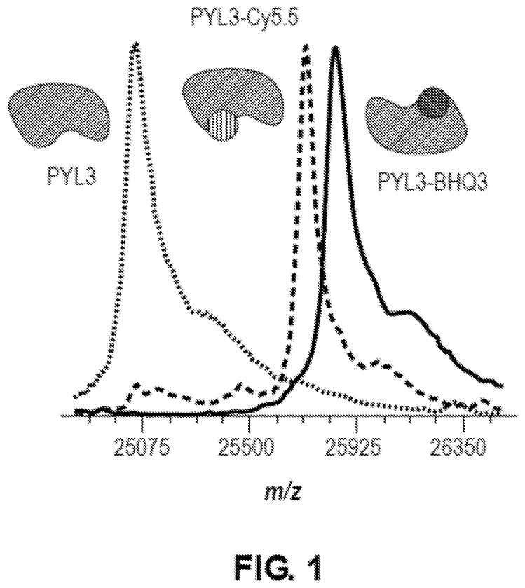

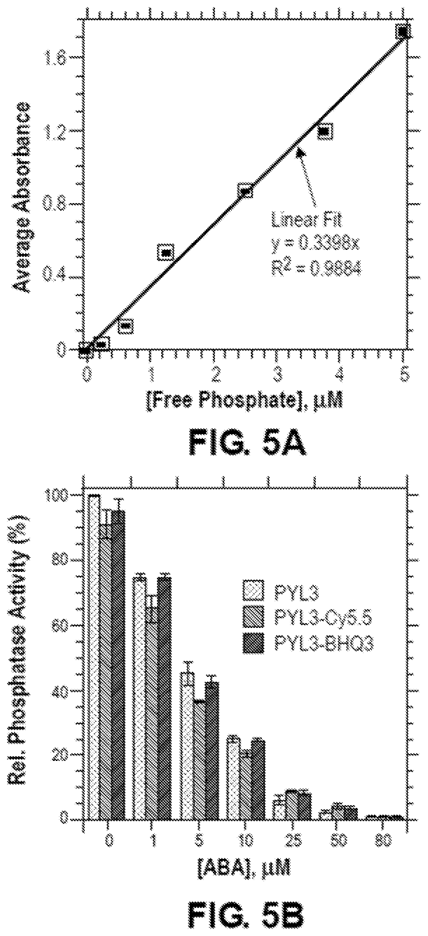

After expression and purification of PYL3, the fluorophore conjugates were subsequently synthesized. PYL3 activity was tested and confirmed using a phosphatase assay, indicating that recombinant protein dye conjugates retained activity. (Hao et al., The Molecular Basis of ABA-Independent Inhibition of PP2Cs by a Subclass of PYL Proteins. Mol Cell 2011, 42 (5), 662-672) Results of the activity assay are shown in FIGS. 5A and 5B. Results of MALDI measurements of post-conjugation revealed three PYL3 protein complexes (FIG. 1)—purified PYL3 (blue line), PYL3-Cy5.5 (red line) and BHQ3-PYL3 (black line). On average only one Cy5.5 and one BHQ3 was conjugated to PYL3, respectively, at the relative concentration ratios of Cy5.5 and BHQ3 to PYL3 employed in this work. MALDI favored the detection of Cy5.5-PYL3 or BHQ3-PYL3 over PYL3 due to high absorption of the desorption laser light by the dye-conjugated proteins. Using the calibrated absorption of the proteins and dyes shown later, the estimated reaction yields was 20% for both protein conjugates. These conjugations do not hinder dimerization or ABA dissociation of dimers, as observed with the phosphatase assay. The success of conjugating PYL3 to both Cy5.5 and BHQ3 suggests that the fluorophore/quencher binding sites do not interfere with the functionalities of PYL3.

When PYL3-Cy5.5 or PYL3-BHQ3 are dissolved in water, they form PYL3-Cy5.5 or PYL3-BHQ3 homodimers. For convenience, we call PYL3-Cy5.5 dimers or PYL3-BHQ3 dimers “homodimers”. UV-Vis absorption measurements covering wavelengths from 270 nm to 700 nm reveal absorption spectra of PYLs (peaked at 280 nm) and Cy5.5 and BHQ3 (peaked at 650 nm) (FIG. 2A). Absorption at 280 nm was used to determine the amount of PYL3 and the absorption near 650 nm was used to gauge the amount of Cy5.5 and BHQ3 in PYL3, respectively. Bases on the calibrated absorption by PYL3 at 280 nm and by pure Cy5.5 and BHQ3 NHS esters (not shown) at 650 nm, the yield of Cy5.5-PYL3 or BHQ3-PYL3 was found to be 20%.

Fluorescence from PYL3 and BHQ3-PYL3 homodimers in aqueous solution is weak, whereas fluorescence from PYL3-Cy5.5 homodimers (FIG. 2B) was as similarly intense as that of Cy5.5. Furthermore, fluorescence from PYL3-Cy5.5 in aqueous solution was found to be partially quenched due to self-quenching, i.e., by the adjacent Cy5.5 molecules in PYL3-Cy5.5 homodimers, similar to the reduced fluorescence in the solution of high concentrations of fluorophores or high density of fluorophores on the surface of nanoparticles. (Reineck et al., Distance and Wavelength Dependent Quenching of Molecular Fluorescence by Au@SiO2 Core-Shell Nanoparticles. Acs Nano 2013, 7 (8), 6636-6648; Chen et al., Fluorescence Self-Quenching from Reporter Dyes Informs on the Structural Properties of Amyloid Clusters Formed in Vitro and in Cells. Nano Lett 2017, 17 (1), 143-149) As ABA is added, fluorescence from PYL3-Cy5.5 increased, confirming the existence of self-quenching. FIG. 2C shows the results of a test of PYL3-Cy5.5 responses to the addition of ABA. If there is no change to the fluorescence intensity, then no self-quenching occurs. As shown in FIG. 2C, on average fluorescence intensity increases by 20% as ABA increases from 0.1 μM to 1 mM, suggesting at least 20% self-quenching in the PYL3-Cy5.5 homodimers in the absence of ABA in aqueous solution. This quenching efficiency was used to estimate the quenching efficiency between Cy5.5 and BHQ3 in PYL3 heterodimers.

Results from Static Light Scattering (SLS) measurements were used to estimate average molecular mass (FIG. 2D) based on molecular motion in solution, although the method is controversial when the molecular mass is on the order of a few thousand atomic mass units. (Oberlerchner et al., Overview of Methods for the Direct Molar Mass Determination of Cellulose. Molecules 2015, 20 (6), 10313-10341) All three proteins respond to ABA similarly, again suggesting that dye-conjugated PYL3 proteins function similarly to PYL3 in terms of binding to ABA. The average molecular mass of the species in solution decreases by nearly 20% as the ABA concentration increases from 100 nM to 40 μM, and another 20% as ABA concentration increases from 50 to 100 μM. This means that more PYL3-Cy5.5 homodimers dissociate as more ABA is added to the solution of PYL3-Cy5.5. The SLS results show an average molecular mass of 50 kDa at 0.1 μM ABA and 30 kDa at 1 mM ABA. If a molecular mass of 51.6 kDa is chosen for homodimers and 25.8 kDa for monomers, then at 0.1 μM ABA 6% of the PYL-Cy5.5 in solution is in the monomeric form, meaning 94% of PYL3-Cy5.5 is in the homodimer form. At 1 mM ABA, 76% of the PYL3-Cy5.5 was observed to be in the monomeric form and 24% of the PYL3-Cy5.5 stayed in the homodimer form. These results are shown in FIG. 2D.

By mixing PYL3-Cy5.5 molecules with PYL3-BHQ3 molecules at an approximately 1:1 ratio in aqueous solutions, monomers, homodimers, and heterodimers coexist, as displayed in FIG. 6. We call PYL3-Cy5.5/PYL3-BHQ3 dimers “heterodimers” to distinguish them from homodimers of PYL3/PYL3, PYL3-Cy5.5/PYL3-Cy5.5, or PYL3-BHQ3/PYL3-BHQ3. UV-Vis spectroscopy was used to determine the equivalent concentrations of the proteins in solution (FIG. 3A). The fluorescence, recorded as background or noise without ABA, comes from PYL3-Cy5.5, which belong to PYL3-Cy5.5 monomers, PYL3-Cy5.5 homodimers and PYL3-Cy5.5/PYL3-BHQ3 heterodimers (FIG. 3B). Without ABA or at low ABA concentrations, most PYL3-Cy5.5 and PYL3-BHQ3 exist as dimers, similar to PYL3 or PYL3-Cy5.5. The measured fluorescence, therefore, is lower than the fluorescence from monomers because of self-quenching, quenching by BHQ3 in heterodimers through FRET as well as PYL3-BHQ3 monomers through attenuation.

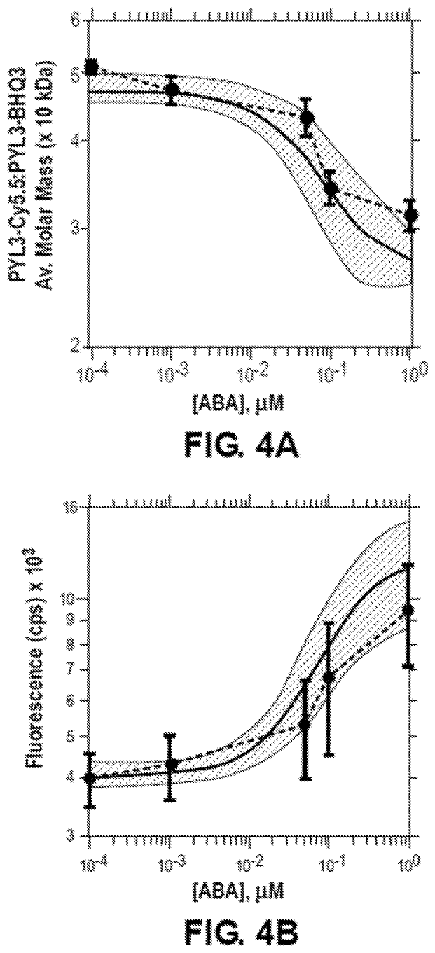

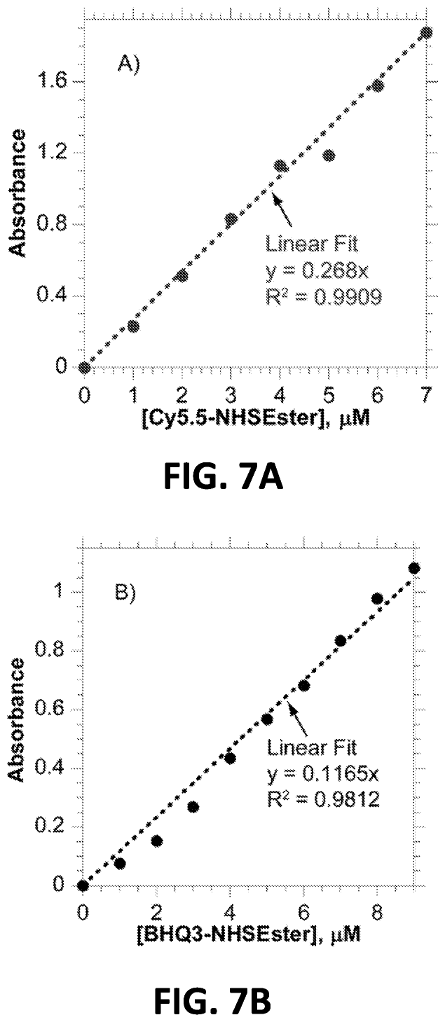

Because of these fluorescence reduction possibilities, it can be difficult to use fluorescence to directly measure the concentration of PYL-Cy5.5, and the use of absorption spectroscopy can instead be a more reliable way to determine the conjugates concentration, which is also subjected to interference from concentration-dependent spectral changes. Using the absorbance values at 280 and 643 nm for PYL3-Cy5.5 and PYL3-BHQ3 shown in FIG. 3A and the absorbance values for Cy5.5 and BHQ3 aqueous solutions (FIGS. 7A and 7B), and if the conjugated Cy5.5 and BHQ3 absorb identically as in PYL3 proteins, the concentrations of Cy5.5 and BHQ3 in the samples shown in FIG. 4A were calculated to be 4.65 and 4.00 μM, respectively.

BHQ3-induced attenuation by free BHQ3 was investigated here. As the concentration of free BHQ3 and Cy5.5 increases in a mixture, attenuation of Cy5.5 fluorescence by BHQ3 is observed (FIG. 3C), which is caused by the absorption of fluorescence from Cy5.5 by BHQ3 (FIG. 3D). As a result, in presence of BHQ3, Cy5.5 fluorescence is attenuated, even without FRET-based quenching. Using the data shown in FIG. 2, the concentrations of Cy5.5 and BHQ3 in the samples were calculated to be 4.65 and 4.00 μM, respectively. Based on the data shown in FIG. 3C, there is nearly 64% attenuation to the fluorescence of 4 μM Cy5.5 by 4 μM BHQ3, decreasing the fluorescence intensity from 72,000 counts per second (cps) from pure Cy5.5 to 26,000 cps for the mixture of Cy5.5 and BHQ3. This attenuation algorithm will be applied to explaining the results from studying PYL-bound Cy5.5 and BHQ3.

SLS was used to quantify the percentages of heterodimers as a function of ABA concentration (black line) (FIG. 3E). A 50 kDa average molecule mass was observed at 100 nM ABA, which corresponds to 98% of the PYL3 or conjugates being in the dimeric form, among which 25% are PYL3-BHQ3 homodimers, 25% are PYL3-Cy5.5 homodimers, and 50% are PYL3-BHQ3 and PYL3-Cy5.5 heterodimers (FIG. 6). As observed with adding ABA to PYL3-Cy5.5 solutions, the amount of monomer increased upon addition of ABA. At 1 mM ABA, 80% of the PYL3 or conjugates are in the monomeric form. Table 1 shows the percentages of monomers and dimers, including both hetero- and homodimers, and the corresponding average molar masses at varying ABA concentrations calculated using the SLS data.

The fluorescence signal (red line) of the mixture increases as ABA is added (FIG. 3E). This increase should arise from more dissociated PYL3-Cy5.5/PYL3-BHQ3 monomers, which lead to decreased FRET quenching of Cy5.5 by BHQ3 in the heterodimers and decreased self-quenching from Cy5.5 in PYL3-Cy5.5/PYL3-Cy5.5 homodimers as shown in FIG. 2C. Assuming BHQ3 in PYL3-BHQ3 behaves similarly as free BHQ3 in solutions, BHQ3 concentration dependent attenuation (FIG. 3D) is 64%, i.e., fluorescence is decreased to 36% of the signal of pure Cy5.5, at the BHQ3-PYL3 concentration of 4.0 μM used in the measurements. FRET quenching of Cy5.5 by BHQ3 in heterodimers is assumed to be 90%. Table 1 shows the estimated fluorescence intensities at varying ABA concentrations using the quenching efficiencies given above. In calculating the final fluorescence intensities (Total fluorescence) in Table 1, we employ the self-quenching, FRET quenching and BHQ3 concentration-dependent attenuation efficiency of 20%, 90% and 73.2%, respectively. The corresponding factors are 0.8, 0.1 and 0.27, respectively. The BHQ3 concentration dependent attenuation efficiency is slightly higher than 64% measured using a mixture of 4 μM of Cy5.5 and 4 μM BHQ3. The calculated final fluorescence intensities are within the standard deviations of the measured values.

TABLE 1
Calculated percentages of dimers and monomers at different ABA
concentrations.

| ABA Concentration (μM) | 0.1 | 1.0 | 40 | 100 | 1000 |
|---|---|---|---|---|---|
| Average molar mass (measured, amu) | 51110 | 47410 | 43330 | 34050 | 31080 |
| PYLs in dimers (hetero- and homodimers, %) | 98 | 84 | 68 | 32 | 20 |
| PYLs in monomers (%) | 2 | 16 | 32 | 68 | 80 |
| Fluorescence (measured) | 3960 | 4292 | 5301 | 6711 | 9494 |
| Fluorescence from Cy5.5 in monomers^{a} | 630 | 5040 | 10080 | 21420 | 25200 |
| Fluorescence from Cy5.5 in homodimers^{b} | 12348 | 10584 | 8568 | 4032 | 2520 |
| Fluorescence from Cy5.5 in heterodimers^{c} | 1575 | 1350 | 1092 | 514 | 315 |
| Sum of Cy5.5 fluorescence^{d} | 14553 | 16974 | 19741 | 25866 | 28035 |
| Total fluorescence^{e} | 4045 | 4718 | 5488 | 7218 | 7793 |

For the data presented in Table 1, a self-quenching efficiency of 20% and BHQ3 concentration dependent quenching shown in FIG. 3C are used for calculation together with a calibrated fluorescence intensity of 79,653 cps from 4.65 μM Cy5.5. Units of fluorescence intensity are counts per second (cps). The values are calculated unless specified otherwise.^{a} Fluorescence intensity=amount of Cy5.5×calibrated unit fluorescence intensity.^{b} Fluorescence intensity=amount of Cy5.5×self-quenching factor (0.8)×calibrated unit fluorescence intensity.^{c} Fluorescence intensity=amount of Cy5.5×FRET quenching factor (0.1)×calibrated unit fluorescence intensity.^{d} Sum of a, b, and c.^{e} Total fluorescence=BHQ3 concentration attenuation factor (0.27)×d.

In order to further evaluate the validity of the measured results, we have carried out theoretical simulations to predict equilibrium concentrations of monomers and dimers based on the rate equations and binding constants. FIGS. 4A and 4B display results of simulations of binding and dissociation dynamics. The yield of conjugation is again assumed to be 100%, and the concentrations of PYL3-Cy5.5 and PYL-BHQ3 are 40 μM. Although conjugated PYL3 is used in the modeling, as we have shown that conjugation has minimal influence on dimer formation or ABA binding, the results should be identical to PYL3. The kinetic rate equations containing the species of interest are shown in the SI. The rate constants are either obtained in the literature or assumed in this work. Regarding the assumed constants, they are varied to examine and obtain the response. FIG. 4A (solid line) shows the average molecular masses of mixtures for different combinations of rate constants and ABA concentrations. The trend obtained from theoretical simulations closely resembles that of the experimental values (dashed line and round circles) that are also shown in FIG. 3C (black line). The shaded area shows the range of average masses as the rate constants are changed. FIG. 4B (solid line) shows the total fluorescence intensity of different combinations of rate constants, with the signal being the sum of the rate constants of PYL3-Cy5.5, PYL3-Cy.5-ABA, PYL3-Cy5.5/PYL3-Cy5.5, and PYL3-Cy5.5/PYL3-BHQ3. Attenuation from BHQ3-PYL3 at 4 μM is also taken into consideration. The fluorescence ABA dependency is similar in all cases, i.e., fluorescence stays relatively constant at very low ABA concentrations, below 1-10 μM. A semi-linear relationship is established once the ABA concentration is between 10 and 1000 μM. This general trend obtained from theoretical simulation agrees with the experimentally acquired dimer-ABA concentration profile pictured in FIG. 3E (red line). FIG. 4A (caption) presents the rate constants. It is noticeable that k₁ through k₃ are quite different from k₋₄, like k₄ is from k₋₁ through k₋₃. This can be explained as k₁ through k₃ are for dissociation of dimers in presence of ABA whereas k₄ is for dissociation without ABA. Therefore, k₋₄ is equivalent to k₁ through k₃ multiplying the ABA concentration, which is on the order of μM. Therefore, k₋₄ is nearly 5 orders of magnitude smaller than k₁ through k₃. This allows to conclude that without ABA, most PYL3 proteins are in the dimeric form.

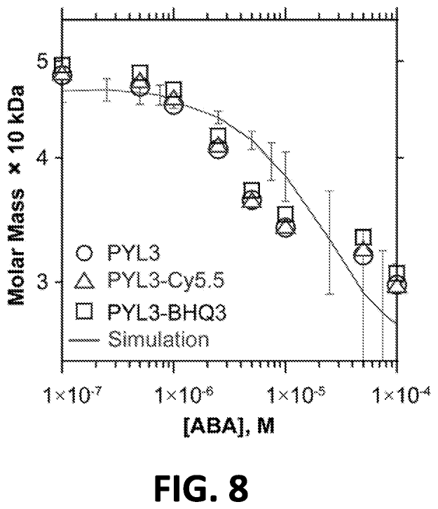

FIG. 8 shows the results of the average molar mass detection using 1.9 μM PYL3-Cy5.5 and PYL3-BHQ3 as a function of ABA concentrations. The inflection point of the transition is around 4 μM of ABA, which is much lower than the 75 μM using 40 μM PYL3-Cy5.5/PYL3-BHQ3 shown in FIG. 3E. Simulated results using the same set of rate constants as in FIGS. 4A and 4B agree with the experimental results. Although only average molar mass results are shown, the fluorescence data can be expected to follow the same anti-correlation trend as that shown in FIG. 3E.

### Discussion

Here, we present a novel approach that can sense ABA through FRET between Cy5.5 and BHQ3 conjugated PYL3 monomers. The current design can detect μM concentrations of ABA in aqueous solutions. The advantages are twofold: 1) a single dissociation reaction is needed to enable the sensing and 2) detection of fluorescence in a single wavelength region is needed. The results presented here demonstrate a proof-of-principle sensing mechanism. Several improvements can be made. For example, FRET quenching efficiency may be maximized. Our conjugation method does not control for the specific amino acid location of Cy5.5 or BHQ3 conjugation. There are many amine sites on the surface of PYL3 monomers. All these sites could potentially be the binding site(s) for Cy5.5 or BHQ3. The random position of fluorophore/quenching conjugation may lead to less efficient FRET quenching. Quenching depends on many parameters including the distance between the two chromophores and their relative orientations. If this quenching is 100% efficient, then one can adjust the system to maximize self-quenching efficiency. If there is strong self-quenching and 100% FRET quenching between Cy5.5 in homodimers, then the fluorescence signal prior to ABA binding can be minimized such that the increase in fluorescence signal upon ABA binding would be greater, hence increasing the detection sensitivity. In order to construct sensors which can be applied, in some cases one can use fluorophore-quencher combinations that emit in the near infrared region to avoid background interference due to chlorophyll emission, such as Cy7.5.

In addition, the cost and shelf lifetime of the sensors can be further improved. Currently PYL protein expression and purification is carried out using E. coli and the conjugation synthesis process is labor intensive. In the future, although unlike, dye-conjugated proteins may be synthesized in plants. This would significantly reduce the cost and eliminate the issue of protein activity lifetime.

Although the trends shown in the simulated results displayed in FIGS. 4A and 4B are in general agreement with the experimentally observed data, the measured SLS data still in part differ from the theoretically simulated results. The difference could be caused by using the SLS method to determine the average molar mass. The method has intrinsic errors in determination of the molecular weight of monomers that have molecular weights of less than 30,000 g mol⁻¹. (Oberlerchner et al., Overview of Methods for the Direct Molar Mass Determination of Cellulose. Molecules 2015, 20 (6), 10313-10341) A full calibration may be required for all the percentages of monomers and dimers and for all the conjugated as well as non-conjugated dimers. The simulations predicted a more complete dissociation than the experimentally measured values. These values were calculated using both fluorescence and molar mass, suggesting that this discrepancy does not depend on quenching calculations. Instead, experimentally, there are dimers that do not dissociate at the highest ABA concentrations, suggesting that some PYL3 may not be able to bind ABA.

Additionally, the signal-to-noise ratios (SNR) can be improved prior to deployment in plants. The SNR obtained here are relatively low, at approximately 5.0 because the data acquisition time per data point was on the order of 5 seconds (FIG. 3E). In one of the published reports on using two wavelength detection, a <2% standard deviation (STD) was obtained, which suggests that SNR on the order of 50 can be obtained. (Waadt et al., FRET-based reporters for the direct visualization of abscisic acid concentration changes and distribution in Arabidopsis. Elife 2014, 3) Given that our current design can detect 200 nM ABA (FIG. 3D), it is reasonable to anticipate that further optimization will enable detection of lower, more physiologically relevant concentrations of ABA.

### Conclusions

A biosensor that uses native PYL3 proteins conjugated with Cy5.5 fluorophores and BHQ3 quenchers has been developed and tested here. Conjugation reaction yield is on the order of 20%, meaning approximately 20% of PYL3 proteins have fluorophores or quenchers on them. Mixture of equal amounts of PYL3-Cy5.5 and PYL3-BHQ3 protein conjugates is used as the biosensor. Fluorescence signal of the mixture increases by more than 50% upon mixing with 100 μM ABA, a phytohormone molecule that is produced in plants undergoing drought stress. Without ABA, most PYL3 conjugates stay in the dimer form, and the fluorescence of Cy5.5 is quenched due to three types of quenching: FRET through BHQ3, self, and concentration dependent through BHQ3. The magnitudes of these quenching are on the order of 90%, 20% and 73% for the concentrations (˜4 μM) of BHQ3 and Cy5.5 used in this work. Upon binding to ABA, dimers, including both PYL3-Cy5.5/PYL3-BHQ3 heterodimers and PYL3-Cy5.5/PYL3-BHQ3 homodimers, dissociate to give rise to higher fluorescence intensities. The experimentally observed responses generally agree with the theoretically model trends.

### Additional Results

TABLE 2
Rate constants for reaction simulation

| k₁ | k₋₁ | k₂ (M⁻¹) | k₋₂ (M) | k3 (M) | k₋₃ (M⁻¹) | k4 (M⁻¹) | k₋₄ (M) |
|---|---|---|---|---|---|---|---|
| 1.5-1.8 | 0.6-0.9 | 1.5-1.8 | 0.6-0.9 | 0.6-0.9 | 1.5-1.8 | 9.0-15.0 | 7.2 × 10⁻⁶-5. × 10⁻⁵ |

### Phosphatase Activity Assay

The phosphatase activity assay was performed using non-radioactive Serine/Threonine Phosphatase Assay System from Promega. (Promega Technical Bulletin, Serine/Threonine Phosphatase Assay System: Instructions for Use of Products V2460 (Revised 3/17, TB218), Promega Corporation, Madison, Wis.; Yin et al., Structural insights into the mechanism of abscisic acid signaling by PYL proteins. Nature Str. and Mol. Biol. 2009, 16 12), 1230-1236); Zhang et al., Complex Structures of the Abscisic Acid Receptor PYL3/RCAR13 Reveal a Unique Regulatory Mechanism. Structure 2012, 20, 780-790) The reaction was performed in a 100-μl reaction volume containing 14 μg ABI1, 20 mM Tris buffer at a pH 7.5, and 150 mM NaCl and 20 μg PYL3. The assay was performed with and without 10 μM (+)-ABA. The reaction mixture was then mixed with Promega supplied peptide substrate in reaction buffer (50 mM imidazole at pH 7.2, 5 mM MgCl₂, 0.2 mM EGTA, and 0.1 mg/mL BSA) at 23° C. for 40 min. The reaction was then terminated by addition of 100 μL molybdate dye/additive mixture. 30 min. after the addition of molybdate dye/additive mixture the phosphate concentration was determined by measuring absorbance at 630 nm and comparing it to the standard calibration curve prepared using Promega supplied free phosphate standard and phosphate-free water. Molybdate dye binds to free phosphate in aqueous solution in a free phosphate concentration dependent manner. To determine relative phosphate activity (%) first the concentration of free phosphate present in PYL3, PYL3-Cy5.5, and PYL3-BHQ3 was calculated for different concentrations of ABA using the standard calibration curve. Secondly, the phosphatase activity was assumed as 100% for PYL3 at 0 μM ABA. Third, relative phosphatase activity (%) at various concentrations of ABA for each of PYL3, PYL3-Cy5.5, and PYL3-BHQ3 was then calculated using PYL3 at 0 μM ABA as reference.

### Reaction Steps Used for Theoretical Simulation

The following multi-step reaction was used to perform theoretical simulation with a range of equilibrium constants in order to validate the observed experimental results in Mathematica. (Toth, J.; Nagy, A. L.; and Papp, D. Reaction Kinetics—A Mathematica Package with Applications. Chem. Eng. Sci. 2012, 83, 12-23).

$$PFPF + ABA {{⇄}_{{k}_{- 1}}}^{{k}_{1}} PQ - ABA + PF$$

$$PQPQ + ABA {{⇄}_{{k}_{- 1}}}^{{k}_{1}} PQ - ABA + PQ$$

$$PQPF + ABA {{⇄}_{{k}_{- 1}}}^{{k}_{1}} PQ - ABA + PF$$

$$PQPF + ABA {{⇄}_{{k}_{- 1}}}^{{k}_{1}} PF - ABA + PQ$$

$$PF + ABA {{⇄}_{{k}_{- 2}}}^{{k}_{2}} PF - ABA$$

$$PQ + ABA {{⇄}_{{k}_{- 2}}}^{{k}_{2}} PQ - ABA$$

$$PF - ABA + PF - ABA {{⇄}_{{k}_{- 3}}}^{{k}_{3}} PFPF + 2 ABA$$

$$PQ - ABA + PQ - ABA {{⇄}_{{k}_{- 3}}}^{{k}_{3}} PQPQ + 2 ABA$$

$$PF - ABA + PQ - ABA {{⇄}_{{k}_{- 3}}}^{{k}_{3}} PFPQ + 2 ABA$$

$$PF + PF {{⇄}_{{k}_{- 4}}}^{{k}_{4}} PFPF$$

$$PQ + PQ {{⇄}_{{k}_{- 4}}}^{{k}_{4}} PQPQ$$

$$PQ + PF {{⇄}_{{k}_{- 4}}}^{{k}_{4}} PQPF$$

$$\frac{d ( PFPF )}{dt} = {k}_{1} ( PFPF \times ABA ) + {k}_{- 1} ( PF - ABA \times PF ) + {{k}_{3} ( PF - ABA )}^{2} - {k}_{- 3} ( PFPF + {( ABA )}^{2} - {k}_{4} ( PFPF ) + {{k}_{- 4} ( PF )}^{2} \\ \frac{d ( PQPQ )}{dt} = {k}_{1} ( PQPQ \times ABA ) + {k}_{- 1} ( PQ - ABA \times PQ ) + {{k}_{3} ( PQ - ABA )}^{2} - {k}_{- 3} ( PQPQ + {( ABA )}^{2} - {k}_{4} ( PQPQ ) + {{k}_{- 4} ( PQ )}^{2} \\ \frac{d ( PQPF )}{dt} = {k}_{1} ( PQPF \times ABA ) + {k}_{- 1} ( PQ - ABA \times PF ) + {{k}_{3} ( PF - ABA )}^{2} - {k}_{3} ( PFPF + {( ABA )}^{2} - {k}_{4} ( PFPF ) + {{k}_{4} ( PF )}^{2} \\ \frac{d ( PF - ABA )}{dt} = {k}_{1} ( PFPF \times ABA ) + {k}_{- 1} ( PQ - ABA \times PF ) + {k}_{1} ( PQPF \times ABA ) - {k}_{- 1} ( PF - ABA \times PQ ) - {k}_{2} ( PF + ABA ) - {k}_{- 2} ( PF - ABA ) - {{k}_{3} ( PF - ABA )}^{2} + {{k}_{- 3} ( PF - ABA )}^{2} + {k}_{3} ( PFPF \times {( ABA )}^{2} ) - {k}_{- 3} ( PF - ABA \times PQ - ABA ) + {k}_{3} ( PQPF \times {( ABA )}^{2} ) \\ \frac{d ( PQ - ABA )}{dt} = {k}_{1} ( PQPQ \times ABA ) - {k}_{- 1} ( PQ - ABA \times PQ ) + {k}_{1} ( PQPF \times ABA ) - {k}_{- 1} ( PQ - ABA \times PF ) + {k}_{2} ( PQ \times ABA ) - {k}_{- 2} ( PQ - ABA ) - {{k}_{3} ( PQ - ABA )}^{2} + {k}_{- 3} ( PQPQ \times {( ABA )}^{2} + {k}_{3} ( PFPF \times {( ABA )}^{2} ) - {k}_{3} ( PF - ABA \times PQ - ABA ) + {k}_{- 3} ( PQPF \times {( ABA )}^{2} ) \\ \frac{d ( PF )}{dt} = {k}_{1} ( PFPF \times ABA ) + {k}_{- 1} ( PF - ABA \times PF ) + {k}_{1} ( PQPF \times ABA ) - {k}_{- 1} ( PQ - ABA \times PF ) - {k}_{2} ( PF \times ABA ) + {k}_{- 2} ( PF - ABA ) + {k}_{4} ( PFPF ) - {{k}_{- 4} ( PF )}^{2} + {k}_{4} ( PQPF ) - {k}_{- 4} ( PQ \times PF ) \\ \frac{d ( PQ )}{dt} = {k}_{1} ( PQPQ \times ABA ) + {k}_{- 1} ( PQ - ABA \times PQ ) + {k}_{1} ( PQPF \times ABA ) - {k}_{- 1} ( PF - ABA \times PQ ) - {k}_{2} ( PQ \times ABA ) + {k}_{- 2} ( PQ - ABA ) + {k}_{4} ( PQPQ ) - {{k}_{- 4} ( PQ )}^{2} + {k}_{4} ( PQPF ) - {k}_{- 4} ( PQ \times PF ) \\ \frac{d ( ABA )}{dt} = {k}_{1} ( PFPF \times ABA ) + {k}_{- 1} ( PF - ABA \times PF ) - {k}_{1} ( PQPQ \times ABA ) + {k}_{- 1} ( PQ - ABA \times PQ ) - {k}_{1} ( PQPF \times ABA ) + {k}_{- 1} ( PQ - ABA \times PF ) - {k}_{1} ( PQPF \times ABA ) + {k}_{- 1} ( PF - ABA \times PQ ) - {k}_{2} ( PQ \times ABA ) - {k}_{- 2} ( PQ - ABA ) - {k}_{2} ( PF \times ABA ) + {k}_{- 2} ( PQ - ABA ) - {k}_{2} ( PF \times ABA ) + {k}_{- 2} ( PF - ABA ) + {{k}_{3} ( PF - ABA )}^{2} + {k}_{- 3} ( PFPF \times {( ABA )}^{2} ) + {{k}_{3} ( PQ - ABA )}^{2} - {k}_{- 3} ( PQPQ \times {( ABA )}^{2} ) + {k}_{3} ( PF - ABA \times PQ - ABA ) - {k}_{- 3} ( PQPF \times {( ABA )}^{2} )$$

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

| | |
|---|---|
| <210> | 1 |
| <211> | 191 |
| <212> | PRT |
| <213> | Arabidopsis thaliana |
| <220> | |
| <223> | thale cress PYR/PYL receptor, Pyrabactin resistance 1, |
| | abscisic acid receptor PYR1 (PYR1), ABI1-binding protein 6 |
| | (ABIP6), regulatory components of ABA receptor 11 (RCAR11), |
| | At4g17870, T6K21.50 |
| <400> | 1 |

```
Met Pro Ser Glu Leu Thr Pro Glu Glu Arg Ser Glu Leu Lys Asn Ser
  1 5 10 15
Ile Ala Glu Phe His Thr Tyr Gln Leu Asp Pro Gly Ser Cys Ser Ser
                        20 25 30
Leu His Ala Gln Arg Ile His Ala Pro Pro Glu Leu Val Trp Ser Ile
                35 40 45
Val Arg Arg Phe Asp Lys Pro Gln Thr Tyr Lys His Phe Ile Lys Ser
          50 55 60
Cys Ser Val Glu Gln Asn Phe Glu Met Arg Val Gly Cys Thr Arg Asp
65 70 75 80
Val Ile Val Ile Ser Gly Leu Pro Ala Asn Thr Ser Thr Glu Arg Leu
                                85 90 95
Asp Ile Leu Asp Asp Glu Arg Arg Val Thr Gly Phe Ser Ile Ile Gly
                        100 105 110
Gly Glu His Arg Leu Thr Asn Tyr Lys Ser Val Thr Thr Val His Arg
                115 120 125
Phe Glu Lys Glu Asn Arg Ile Trp Thr Val Val Leu Glu Ser Tyr Val
        130 135 140
Val Asp Met Pro Glu Gly Asn Ser Glu Asp Asp Thr Arg Met Phe Ala
145 150 155 160
Asp Thr Val Val Lys Leu Asn Leu Gln Lys Leu Ala Thr Val Ala Glu
                                165 170 175
Ala Met Ala Arg Asn Ser Gly Asp Gly Ser Gly Ser Gln Val Thr
                        180 185 190
```

| | |
|---|---|
| <210> | 2 |
| <211> | 221 |
| <212> | PRT |
| <213> | Arabidopsis thaliana |
| <220> | |
| <223> | thale cress PYR/PYL receptor, abscisic acid receptor PYL1, |
| | PYR1-like protein 1 (PYL1), ABI1-binding protein 6 (ABIP6), |
| | regulatory components of ABA receptor 9 (RCAR12), |
| | At5g46790, MZA15.21 |
| <400> | 2 |

```
Met Ala Asn Ser Glu Ser Ser Ser Ser Pro Val Asn Glu Glu Glu Asn
  1 5 10 15
Ser Gln Arg Ile Ser Thr Leu His His Gln Thr Met Pro Ser Asp Leu
                        20 25 30
Thr Gln Asp Glu Phe Thr Gln Leu Ser Gln Ser Ile Ala Glu Phe His
                35 40 45
Thr Tyr Gln Leu Gly Asn Gly Arg Cys Ser Ser Leu Leu Ala Gln Arg
          50 55 60
Ile His Ala Pro Pro Glu Thr Val Trp Ser Val Val Arg Arg Phe Asp
65 70 75 80
Arg Pro Gln Ile Tyr Lys His Phe Ile Lys Ser Cys Asn Val Ser Glu
                                85 90 95
Asp Phe Glu Met Arg Val Gly Cys Thr Arg Asp Val Asn Val Ile Ser
                        100 105 110
Gly Leu Pro Ala Asn Thr Ser Arg Glu Arg Leu Asp Leu Leu Asp Asp
                115 120 125
Asp Arg Arg Val Thr Gly Phe Ser Ile Thr Gly Gly Glu His Arg Leu
        130 135 140
Arg Asn Tyr Lys Ser Val Thr Thr Val His Arg Phe Glu Lys Glu Glu
145 150 155 160
Glu Glu Glu Arg Ile Trp Thr Val Val Leu Glu Ser Tyr Val Val Asp
                                165 170 175
Val Pro Glu Gly Asn Ser Glu Glu Asp Thr Arg Leu Phe Ala Asp Thr
                        180 185 190
Val Ile Arg Leu Asn Leu Gln Lys Leu Ala Ser Ile Thr Glu Ala Met
                195 200 205
Asn Arg Asn Asn Asn Asn Asn Asn Ser Ser Gln Val Arg
        210 215 220
```

| | |
|---|---|
| <210> | 3 |
| <211> | 190 |
| <212> | PRT |
| <213> | Arabidopsis thaliana |
| <220> | |
| <223> | thale cress PYR/PYL receptor, abscisic acid receptor PYL2, |
| | PYR1-like protein 2 (PYL2), ABI1-binding protein 6 (ABIP6), |
| | regulatory components of ABA receptor 14 (RCAR14), Bet v I |
| | allergen family protein, At2g26040, T19L18.15 |
| <400> | 3 |

```
Met Ser Ser Ser Pro Ala Val Lys Gly Leu Thr Asp Glu Glu Gln Lys
  1 5 10 15
Thr Leu Glu Pro Val Ile Lys Thr Tyr His Gln Phe Glu Pro Asp Pro
                        20 25 30
Thr Thr Cys Thr Ser Leu Ile Thr Gln Arg Ile His Ala Pro Ala Ser
                35 40 45
Val Val Trp Pro Leu Ile Arg Arg Phe Asp Asn Pro Glu Arg Tyr Lys
          50 55 60
His Phe Val Lys Arg Cys Arg Leu Ile Ser Gly Asp Gly Asp Val Gly
65 70 75 80
Ser Val Arg Glu Val Thr Val Ile Ser Gly Leu Pro Ala Ser Thr Ser
                                85 90 95
Thr Glu Arg Leu Glu Phe Val Asp Asp Asp His Arg Val Leu Ser Phe
                        100 105 110
Arg Val Val Gly Gly Glu His Arg Leu Lys Asn Tyr Lys Ser Val Thr
                115 120 125
Ser Val Asn Glu Phe Leu Asn Gln Asp Ser Gly Lys Val Tyr Thr Val
        130 135 140
Val Leu Glu Ser Tyr Thr Val Asp Ile Pro Glu Gly Asn Thr Glu Glu
145 150 155 160
Asp Thr Lys Met Phe Val Asp Thr Val Val Lys Leu Asn Leu Gln Lys
                                165 170 175
Leu Gly Val Ala Ala Thr Ser Ala Pro Met His Asp Asp Glu
                        180 185 190
```

| | |
|---|---|
| <210> | 4 |
| <211> | 209 |
| <212> | PRT |
| <213> | Arabidopsis thaliana |
| <220> | |
| <223> | thale cress PYR/PYL receptor, abscisic acid receptor PYL3, |
| | PYR1-like protein 3 (PYL3), regulatory components of ABA |
| | receptor 13 (RCAR13), At1g73000, F3N23.20 |
| <400> | 4 |

```
Met Asn Leu Ala Pro Ile His Asp Pro Ser Ser Ser Ser Thr Thr Thr
  1 5 10 15
Thr Ser Ser Ser Thr Pro Tyr Gly Leu Thr Lys Asp Glu Phe Ser Thr
                        20 25 30
Leu Asp Ser Ile Ile Arg Thr His His Thr Phe Pro Arg Ser Pro Asn
                35 40 45
Thr Cys Thr Ser Leu Ile Ala His Arg Val Asp Ala Pro Ala His Ala
          50 55 60
Ile Trp Arg Phe Val Arg Asp Phe Ala Asn Pro Asn Lys Tyr Lys His
65 70 75 80
Phe Ile Lys Ser Cys Thr Ile Arg Val Asn Gly Asn Gly Ile Lys Glu
                                85 90 95
Ile Lys Val Gly Thr Ile Arg Glu Val Ser Val Val Ser Gly Leu Pro
                        100 105 110
Ala Ser Thr Ser Val Glu Ile Leu Glu Val Leu Asp Glu Glu Lys Arg
                115 120 125
Ile Leu Ser Phe Arg Val Leu Gly Gly Glu His Arg Leu Asn Asn Tyr
        130 135 140
Arg Ser Val Thr Ser Val Asn Glu Phe Val Val Leu Glu Lys Asp Lys
145 150 155 160
Lys Lys Arg Val Tyr Ser Val Val Leu Glu Ser Tyr Ile Val Asp Ile
                                165 170 175
Pro Gln Gly Asn Thr Glu Glu Asp Thr Arg Met Phe Val Asp Thr Val
                        180 185 190
Val Lys Ser Asn Leu Gln Asn Leu Ala Val Ile Ser Thr Ala Ser Pro
                195 200 205
Thr
```

| | |
|---|---|
| <210> | 5 |
| <211> | 207 |
| <212> | PRT |
| <213> | Arabidopsis thaliana |
| <220> | |
| <223> | thale cress PYR/PYL receptor, abscisic acid receptor PYL4, |
| | PYR1-like protein 4 (PYL4), ABI1-binding protein 2 (ABIP2), |
| | regulatory components of ABA receptor 10 (RCAR10), |
| | At2g38310, T19C21.20 |
| <400> | 5 |

```
Met Leu Ala Val His Arg Pro Ser Ser Ala Val Ser Asp Gly Asp Ser
  1 5 10 15
Val Gln Ile Pro Met Met Ile Ala Ser Phe Gln Lys Arg Phe Pro Ser
                        20 25 30
Leu Ser Arg Asp Ser Thr Ala Ala Arg Phe His Thr His Glu Val Gly
                35 40 45
Pro Asn Gln Cys Cys Ser Ala Val Ile Gln Glu Ile Ser Ala Pro Ile
          50 55 60
Ser Thr Val Trp Ser Val Val Arg Arg Phe Asp Asn Pro Gln Ala Tyr
65 70 75 80
Lys His Phe Leu Lys Ser Cys Ser Val Ile Gly Gly Asp Gly Asp Asn
                                85 90 95
Val Gly Ser Leu Arg Gln Val His Val Val Ser Gly Leu Pro Ala Ala
                        100 105 110
Ser Ser Thr Glu Arg Leu Asp Ile Leu Asp Asp Glu Arg His Val Ile
                115 120 125
Ser Phe Ser Val Val Gly Gly Asp His Arg Leu Ser Asn Tyr Arg Ser
        130 135 140
Val Thr Thr Leu His Pro Ser Pro Ile Ser Gly Thr Val Val Val Glu
145 150 155 160
Ser Tyr Val Val Asp Val Pro Pro Gly Asn Thr Lys Glu Glu Thr Cys
                                165 170 175
Asp Phe Val Asp Val Ile Val Arg Cys Asn Leu Gln Ser Leu Ala Lys
                        180 185 190
Ile Ala Glu Asn Thr Ala Ala Glu Ser Lys Lys Lys Met Ser Leu
                195 200 205
```

| | |
|---|---|
| <210> | 6 |
| <211> | 203 |
| <212> | PRT |
| <213> | Arabidopsis thaliana |
| <220> | |
| <223> | thale cress PYR/PYL receptor, abscisic acid receptor PYL5, |
| | PYR1-like protein 5 (PYL5), ABI1-binding protein 3 (ABIP3), |
| | regulatory components of ABA receptor 8 (RCAR8), Bet v I |
| | allergen family protein, At5g05440, K18I23.25 |
| <400> | 6 |

```
Met Arg Ser Pro Val Gln Leu Gln His Gly Ser Asp Ala Thr Asn Gly
  1 5 10 15
Phe His Thr Leu Gln Pro His Asp Gln Thr Asp Gly Pro Ile Lys Arg
                        20 25 30
Val Cys Leu Thr Arg Gly Met His Val Pro Glu His Val Ala Met His
                35 40 45
His Thr His Asp Val Gly Pro Asp Gln Cys Cys Ser Ser Val Val Gln
          50 55 60
Met Ile His Ala Pro Pro Glu Ser Val Trp Ala Leu Val Arg Arg Phe
65 70 75 80
Asp Asn Pro Lys Val Tyr Lys Asn Phe Ile Arg Gln Cys Arg Ile Val
                                85 90 95
Gln Gly Asp Gly Leu His Val Gly Asp Leu Arg Glu Val Met Val Val
                        100 105 110
Ser Gly Leu Pro Ala Val Ser Ser Thr Glu Arg Leu Glu Ile Leu Asp
                115 120 125
Glu Glu Arg His Val Ile Ser Phe Ser Val Val Gly Gly Asp His Arg
        130 135 140
Leu Lys Asn Tyr Arg Ser Val Thr Thr Leu His Ala Ser Asp Asp Glu
145 150 155 160
Gly Thr Val Val Val Glu Ser Tyr Ile Val Asp Val Pro Pro Gly Asn
                                165 170 175
Thr Glu Glu Glu Thr Leu Ser Phe Val Asp Thr Ile Val Arg Cys Asn
                        180 185 190
Leu Gln Ser Leu Ala Arg Ser Thr Asn Arg Gln
                195 200
```

| | |
|---|---|
| <210> | 7 |
| <211> | 215 |
| <212> | PRT |
| <213> | Arabidopsis thaliana |
| <220> | |
| <223> | thale cress PYR/PYL receptor, abscisic acid receptor PYL6, |
| | PYR1-like protein 6 (PYL6), ABI1-binding protein 5 (ABIP5), |
| | regulatory components of ABA receptor 9 (RCAR9), Bet v I |
| | allergen family protein, At2g40330, T7M7.15 |
| <400> | 7 |

```
Met Pro Thr Ser Ile Gln Phe Gln Arg Ser Ser Thr Ala Ala Glu Ala
  1 5 10 15
Ala Asn Ala Thr Val Arg Asn Tyr Pro His His His Gln Lys Gln Val
                        20 25 30
Gln Lys Val Ser Leu Thr Arg Gly Met Ala Asp Val Pro Glu His Val
                35 40 45
Glu Leu Ser His Thr His Val Val Gly Pro Ser Gln Cys Phe Ser Val
          50 55 60
Val Val Gln Asp Val Glu Ala Pro Val Ser Thr Val Trp Ser Ile Leu
65 70 75 80
Ser Arg Phe Glu His Pro Gln Ala Tyr Lys His Phe Val Lys Ser Cys
                                85 90 95
His Val Val Ile Gly Asp Gly Arg Glu Val Gly Ser Val Arg Glu Val
                        100 105 110
Arg Val Val Ser Gly Leu Pro Ala Ala Phe Ser Leu Glu Arg Leu Glu
                115 120 125
Ile Met Asp Asp Asp Arg His Val Ile Ser Phe Ser Val Val Gly Gly
        130 135 140
Asp His Arg Leu Met Asn Tyr Lys Ser Val Thr Thr Val His Glu Ser
145 150 155 160
Glu Glu Asp Ser Asp Gly Lys Lys Arg Thr Arg Val Val Glu Ser Tyr
                                165 170 175
Val Val Asp Val Pro Ala Gly Asn Asp Lys Glu Glu Thr Cys Ser Phe
                        180 185 190
Ala Asp Thr Ile Val Arg Cys Asn Leu Gln Ser Leu Ala Lys Leu Ala
                195 200 205
Glu Asn Thr Ser Lys Phe Ser
        210 215
```

| | |
|---|---|
| <210> | 8 |
| <211> | 211 |
| <212> | PRT |
| <213> | Arabidopsis thaliana |
| <220> | |
| <223> | thale cress PYR/PYL receptor, abscisic acid receptor PYL7, |
| | PYR1-like protein 7 (PYL7), ABI1-binding protein 7 (ABIP7), |
| | regulatory components of ABA receptor 2 (RCAR2), At4g01026 |
| <400> | 8 |

```
Met Glu Met Ile Gly Gly Asp Asp Thr Asp Thr Glu Met Tyr Gly Ala
  1 5 10 15
Leu Val Thr Ala Gln Ser Leu Arg Leu Arg His Leu His His Cys Arg
                        20 25 30
Glu Asn Gln Cys Thr Ser Val Leu Val Lys Tyr Ile Gln Ala Pro Val
                35 40 45
His Leu Val Trp Ser Leu Val Arg Arg Phe Asp Gln Pro Gln Lys Tyr
          50 55 60
Lys Pro Phe Ile Ser Arg Cys Thr Val Asn Gly Asp Pro Glu Ile Gly
65 70 75 80
Cys Leu Arg Glu Val Asn Val Lys Ser Gly Leu Pro Ala Thr Thr Ser
                                85 90 95
Thr Glu Arg Leu Glu Gln Leu Asp Asp Glu Glu His Ile Leu Gly Ile
                        100 105 110
Asn Ile Ile Gly Gly Asp His Arg Leu Lys Asn Tyr Ser Ser Ile Leu
                115 120 125
Thr Val His Pro Glu Met Ile Asp Gly Arg Ser Gly Thr Met Val Met
        130 135 140
Glu Ser Phe Val Val Asp Val Pro Gln Gly Asn Thr Lys Asp Asp Thr
145 150 155 160
Cys Tyr Phe Val Glu Ser Leu Ile Lys Cys Asn Leu Lys Ser Leu Ala
                                165 170 175
Cys Val Ser Glu Arg Leu Ala Ala Gln Asp Ile Thr Asn Ser Ile Ala
                        180 185 190
Thr Phe Cys Asn Ala Ser Asn Gly Tyr Arg Glu Lys Asn His Thr Glu
                195 200 205
Thr Asn Leu
        210
```

| | |
|---|---|
| <210> | 9 |
| <211> | 188 |
| <212> | PRT |
| <213> | Arabidopsis thaliana |
| <220> | |
| <223> | thale cress PYR/PYL receptor, abscisic acid receptor PYL8, |
| | PYR1-like protein 8 (PYL8), ABI1-binding protein 1 (ABIP1), |
| | regulatory components of ABA receptor 3 (RCAR3), At5g53160, |
| | MFH8.10 |
| <400> | 9 |

```
Met Glu Ala Asn Gly Ile Glu Asn Leu Thr Asn Pro Asn Gln Glu Arg
  1 5 10 15
Glu Phe Ile Arg Arg His His Lys His Glu Leu Val Asp Asn Gln Cys
                        20 25 30
Ser Ser Thr Leu Val Lys His Ile Asn Ala Pro Val His Ile Val Trp
                35 40 45
Ser Leu Val Arg Arg Phe Asp Gln Pro Gln Lys Tyr Lys Pro Phe Ile
          50 55 60
Ser Arg Cys Val Val Lys Gly Asn Met Glu Ile Gly Thr Val Arg Glu
65 70 75 80
Val Asp Val Lys Ser Gly Leu Pro Ala Thr Arg Ser Thr Glu Arg Leu
                                85 90 95
Glu Leu Leu Asp Asp Asn Glu His Ile Leu Ser Ile Arg Ile Val Gly
                        100 105 110
Gly Asp His Arg Leu Lys Asn Tyr Ser Ser Ile Ile Ser Leu His Pro
                115 120 125
Glu Thr Ile Glu Gly Arg Ile Gly Thr Leu Val Ile Glu Ser Phe Val
        130 135 140
Val Asp Val Pro Glu Gly Asn Thr Lys Asp Glu Thr Cys Tyr Phe Val
145 150 155 160
Glu Ala Leu Ile Lys Cys Asn Leu Lys Ser Leu Ala Asp Ile Ser Glu
                                165 170 175
Arg Leu Ala Val Gln Asp Thr Thr Glu Ser Arg Val
                        180 185
```

| | |
|---|---|
| <210> | 10 |
| <211> | 187 |
| <212> | PRT |
| <213> | Arabidopsis thaliana |
| <220> | |
| <223> | thale cress PYR/PYL receptor, abscisic acid receptor PYL9, |
| | PYR1-like protein 9 (PYL9), ABI1-binding protein 4 (ABIP4), |
| | regulatory components of ABA receptor 1 (RCAR1), At1g01360, |
| | F6F3.16 |
| <400> | 10 |

```
Met Met Asp Gly Val Glu Gly Gly Thr Ala Met Tyr Gly Gly Leu Glu
  1 5 10 15
Thr Val Gln Tyr Val Arg Thr His His Gln His Leu Cys Arg Glu Asn
                        20 25 30
Gln Cys Thr Ser Ala Leu Val Lys His Ile Lys Ala Pro Leu His Leu
                35 40 45
Val Trp Ser Leu Val Arg Arg Phe Asp Gln Pro Gln Lys Tyr Lys Pro
          50 55 60
Phe Val Ser Arg Cys Thr Val Ile Gly Asp Pro Glu Ile Gly Ser Leu
65 70 75 80
Arg Glu Val Asn Val Lys Ser Gly Leu Pro Ala Thr Thr Ser Thr Glu
                                85 90 95
Arg Leu Glu Leu Leu Asp Asp Glu Glu His Ile Leu Gly Ile Lys Ile
                        100 105 110
Ile Gly Gly Asp His Arg Leu Lys Asn Tyr Ser Ser Ile Leu Thr Val
                115 120 125
His Pro Glu Ile Ile Glu Gly Arg Ala Gly Thr Met Val Ile Glu Ser
        130 135 140
Phe Val Val Asp Val Pro Gln Gly Asn Thr Lys Asp Glu Thr Cys Tyr
145 150 155 160
Phe Val Glu Ala Leu Ile Arg Cys Asn Leu Lys Ser Leu Ala Asp Val
                                165 170 175
Ser Glu Arg Leu Ala Ser Gln Asp Ile Thr Gln
                        180 185
```

| | |
|---|---|
| <210> | 11 |
| <211> | 183 |
| <212> | PRT |
| <213> | Arabidopsis thaliana |
| <220> | |
| <223> | thale cress PYR/PYL receptor, abscisic acid receptor PYL10, |
| | PYR1-like protein 10 (PYL10), ABI1-binding protein 8 (ABIP8), |
| | regulatory components of ABA receptor 4 (RCAR4), At4g27920, |
| | T13J8.30 |
| <400> | 11 |

```
Met Asn Gly Asp Glu Thr Lys Lys Val Glu Ser Glu Tyr Ile Lys Lys
  1 5 10 15
His His Arg His Glu Leu Val Glu Ser Gln Cys Ser Ser Thr Leu Val
                        20 25 30
Lys His Ile Lys Ala Pro Leu His Leu Val Trp Ser Ile Val Arg Arg
                35 40 45
Phe Asp Glu Pro Gln Lys Tyr Lys Pro Phe Ile Ser Arg Cys Val Val
        50 55 60
Gln Gly Lys Lys Leu Glu Val Gly Ser Val Arg Glu Val Asp Leu Lys
65 70 75 80
Ser Gly Leu Pro Ala Thr Lys Ser Thr Glu Val Leu Glu Ile Leu Asp
                                85 90 95
Asp Asn Glu His Ile Leu Gly Ile Arg Ile Val Gly Gly Asp His Arg
                        100 105 110
Leu Lys Asn Tyr Ser Ser Thr Ile Ser Leu His Ser Glu Thr Ile Asp
                115 120 125
Gly Lys Thr Gly Thr Leu Ala Ile Glu Ser Phe Val Val Asp Val Pro
        130 135 140
Glu Gly Asn Thr Lys Glu Glu Thr Cys Phe Phe Val Glu Ala Leu Ile
145 150 155 160
Gln Cys Asn Leu Asn Ser Leu Ala Asp Val Thr Glu Arg Leu Gln Ala
                                165 170 175
Glu Ser Met Glu Lys Lys Ile
                        180
```

| | |
|---|---|
| <210> | 12 |
| <211> | 161 |
| <212> | PRT |
| <213> | Arabidopsis thaliana |
| <220> | |
| <223> | thale cress PYR/PYL receptor, abscisic acid receptor PYL11, |
| | PYR1-like protein 11 (PYL11), regulatory components of ABA |
| | receptor 5 (RCAR5), Bet v I allergen family protein, |
| | At5g45860, K15I22.6 |
| <400> | 12 |

```
Met Glu Thr Ser Gln Lys Tyr His Thr Cys Gly Ser Thr Leu Val Gln
  1 5 10 15
Thr Ile Asp Ala Pro Leu Ser Leu Val Trp Ser Ile Leu Arg Arg Phe
                        20 25 30
Asp Asn Pro Gln Ala Tyr Lys Gln Phe Val Lys Thr Cys Asn Leu Ser
                35 40 45
Ser Gly Asp Gly Gly Glu Gly Ser Val Arg Glu Val Thr Val Val Ser
        50 55 60
Gly Leu Pro Ala Glu Phe Ser Arg Glu Arg Leu Asp Glu Leu Asp Asp
65 70 75 80
Glu Ser His Val Met Met Ile Ser Ile Ile Gly Gly Asp His Arg Leu
                                85 90 95
Val Asn Tyr Arg Ser Lys Thr Met Ala Phe Val Ala Ala Asp Thr Glu
                        100 105 110
Glu Lys Thr Val Val Val Glu Ser Tyr Val Val Asp Val Pro Glu Gly
                115 120 125
Asn Ser Glu Glu Glu Thr Thr Ser Phe Ala Asp Thr Ile Val Gly Phe
        130 135 140
Asn Leu Lys Ser Leu Ala Lys Leu Ser Glu Arg Val Ala His Leu Lys
145 150 155 160
Leu
```

| | |
|---|---|
| <210> | 13 |
| <211> | 159 |
| <212> | PRT |
| <213> | Arabidopsis thaliana |
| <220> | |
| <223> | thale cress PYR/PYL receptor, abscisic acid receptor PYL12, |
| | PYR1-like protein 12 (PYL12), regulatory components of ABA |
| | receptor 6 (RCAR6), Bet v I allergen family protein, |
| | At5g45870, K15I22.7 |
| <400> | 13 |

```
Met Lys Thr Ser Gln Glu Gln His Val Cys Gly Ser Thr Val Val Gln
  1 5 10 15
Thr Ile Asn Ala Pro Leu Pro Leu Val Trp Ser Ile Leu Arg Arg Phe
                        20 25 30
Asp Asn Pro Lys Thr Phe Lys His Phe Val Lys Thr Cys Lys Leu Arg
                35 40 45
Ser Gly Asp Gly Gly Glu Gly Ser Val Arg Glu Val Thr Val Val Ser
        50 55 60
Asp Leu Pro Ala Ser Phe Ser Leu Glu Arg Leu Asp Glu Leu Asp Asp
65 70 75 80
Glu Ser His Val Met Val Ile Ser Ile Ile Gly Gly Asp His Arg Leu
                                85 90 95
Val Asn Tyr Gln Ser Lys Thr Thr Val Phe Val Ala Ala Glu Glu Glu
                        100 105 110
Lys Thr Val Val Val Glu Ser Tyr Val Val Asp Val Pro Glu Gly Asn
                115 120 125
Thr Glu Glu Glu Thr Thr Leu Phe Ala Asp Thr Ile Val Gly Cys Asn
        130 135 140
Leu Arg Ser Leu Ala Lys Leu Ser Glu Lys Met Met Glu Leu Thr
145 150 155
```

| | |
|---|---|
| <210> | 14 |
| <211> | 164 |
| <212> | PRT |
| <213> | Arabidopsis thaliana |
| <220> | |
| <223> | thale cress PYR/PYL receptor, abscisic acid receptor PYL13, |
| | PYR1-like protein 13 (PYL13), regulatory components of ABA |
| | receptor 7 (RCAR7), At4g18620, F28A21.30 |
| <400> | 14 |

```
Met Glu Ser Ser Lys Gln Lys Arg Cys Arg Ser Ser Val Val Glu Thr
  1 5 10 15
Ile Glu Ala Pro Leu Pro Leu Val Trp Ser Ile Leu Arg Ser Phe Asp
                        20 25 30
Lys Pro Gln Ala Tyr Gln Arg Phe Val Lys Ser Cys Thr Met Arg Ser
                35 40 45
Gly Gly Gly Gly Gly Lys Gly Gly Glu Gly Lys Gly Ser Val Arg Asp
        50 55 60
Val Thr Leu Val Ser Gly Phe Pro Ala Asp Phe Ser Thr Glu Arg Leu
65 70 75 80
Glu Glu Leu Asp Asp Glu Ser His Val Met Val Val Ser Ile Ile Gly
                                85 90 95
Gly Asn His Arg Leu Val Asn Tyr Lys Ser Lys Thr Lys Val Val Ala
                        100 105 110
Ser Pro Glu Asp Met Ala Lys Lys Thr Val Val Val Glu Ser Tyr Val
                115 120 125
Val Asp Val Pro Glu Gly Thr Ser Glu Glu Asp Thr Ile Phe Phe Val
        130 135 140
Asp Asn Ile Ile Arg Tyr Asn Leu Thr Ser Leu Ala Lys Leu Thr Lys
145 150 155 160
Lys Met Met Lys
```

| | |
|---|---|
| <210> | 15 |
| <211> | 191 |
| <212> | PRT |
| <213> | Brassica oleracea |
| <220> | |
| <223> | wild cabbage Streptomyces cyclase/dehydrase family protein, |
| | locus tag 40.t00062, GenBank Accession No. ABD65175.1, |
| | GI:89257688 |
| <400> | 15 |

```
Met Pro Ser Gln Leu Thr Pro Glu Glu Arg Ser Glu Leu Ala Gln Ser
  1 5 10 15
Ile Ala Glu Phe His Thr Tyr His Leu Gly Pro Gly Ser Cys Ser Ser
                        20 25 30
Leu His Ala Gln Arg Ile His Ala Pro Pro Glu Ile Val Trp Ser Val
                35 40 45
Val Arg Arg Phe Asp Lys Pro Gln Thr Tyr Lys His Phe Ile Lys Ser
        50 55 60
Cys Ser Val Glu Asp Gly Phe Glu Met Arg Val Gly Cys Thr Arg Ala
65 70 75 80
Val Asn Val Ile Ser Gly Leu Pro Ala Asn Thr Ser Thr Glu Arg Leu
                                85 90 95
Asp Ile Leu Asp Asp Glu Arg Arg Val Thr Gly Phe Ser Ile Ile Gly
                        100 105 110
Gly Glu His Arg Leu Thr Asn Tyr Lys Ser Val Thr Thr Val His Arg
                115 120 125
Phe Glu Lys Glu Arg Arg Ile Trp Thr Val Val Leu Glu Ser Tyr Val
        130 135 140
Val Asp Met Pro Glu Gly Asn Ser Glu Asp Asp Thr Arg Met Phe Ala
145 150 155 160
Asp Thr Val Val Lys Leu Asn Leu Gln Lys Leu Ala Thr Val Thr Glu
                                165 170 175
Ala Met Ala Arg Asn Ala Gly Asp Gly Ser Gly Ala Gln Val Thr
                        180 185 190
```

| | |
|---|---|
| <210> | 16 |
| <211> | 281 |
| <212> | PRT |
| <213> | Brassica oleracea |
| <220> | |
| <223> | wild cabbage Streptomyces cyclase/dehydrase family protein, |
| | locus tag 23.t00047, GenBank Accession No. ABD65631.1, |
| | GI:89274227 |
| <400> | 16 |

```
Met Pro Ser Glu Leu Thr Gln Glu Glu Arg Ser Lys Leu Thr Gln Ser
  1 5 10 15
Ile Ser Glu Phe His Thr Tyr His Leu Gly Pro Gly Ser Cys Ser Ser
                        20 25 30
Leu His Ala Gln Arg Ile His Ala Pro Pro Glu Ile Val Trp Ser Val
                35 40 45
Val Arg Gln Phe Asp Lys Pro Gln Thr Tyr Lys His Phe Ile Lys Ser
        50 55 60
Cys Ser Val Glu Glu Gly Phe Glu Met Arg Val Gly Cys Thr Arg Asp
65 70 75 80
Val Ile Val Ile Ser Gly Leu Pro Ala Asn Thr Ser Thr Glu Arg Leu
                                85 90 95
Asp Met Leu Asp Asp Glu Arg Arg Val Thr Gly Phe Ser Ile Ile Gly
                        100 105 110
Gly Glu His Arg Leu Lys Asn Tyr Lys Ser Val Thr Thr Val His Arg
                115 120 125
Phe Glu Arg Glu Arg Arg Ile Trp Thr Val Val Leu Glu Ser Tyr Val
        130 135 140
Val Asp Met Pro Glu Gly Asn Ser Glu Asp Asp Thr Arg Met Phe Ala
145 150 155 160
Asp Thr Val Val Lys Leu Asn Leu Gln Lys Leu Ala Thr Val Thr Glu
                                165 170 175
Ala Met Ala Arg Asn Ala Gly Asp Gly Arg Gly Ser Arg Glu Thr Thr
                        180 185 190
Cys Arg Glu Ser Phe His Leu Ile Thr Ala Phe Glu Lys Gln Arg Gln
                195 200 205
Ile Thr Glu Pro Thr Val Tyr Gln Asn Pro Pro Tyr His Thr Gly Met
        210 215 220
Thr Pro Glu Pro Arg Thr Ser Thr Val Phe Ile Glu Leu Glu Asp His
225 230 235 240
Arg Thr Leu Pro Gly Asn Leu Thr Pro Thr Thr Glu Glu His Leu Gln
                                245 250 255
Arg Met Tyr Gln Arg Phe Trp Gly Ile Arg Gln Leu Gln Arg Pro Arg
                        260 265 270
Gln Ser Phe Gly Glu Arg Gln Ser Ile
                275 280
```

| | |
|---|---|
| <210> | 17 |
| <211> | 453 |
| <212> | PRT |
| <213> | Vitis vinifera |
| <220> | |
| <223> | wine grape cultivar PN40024 unnamed protein product, locus |
| | tag GSVIVT00015766001, GenBank Accession No. CAO63410.1, |
| | GI:157341954 |
| <400> | 17 |

```
Met Gln Met Lys Tyr Leu Glu Gly Lys Gln Asn Leu Met Glu Glu Lys
  1 5 10 15
Gly Glu Lys Gln Cys Ile Pro Met Asp Leu Ala Val Arg Glu Ala Gln
                        20 25 30
Phe Lys Gly Ser Leu Leu Asp Arg Ile Thr Trp Leu Glu Gln Arg Leu
                35 40 45
His Lys Leu Ser Leu Gln Leu Glu Thr Arg Ser Lys Gln Gln Pro His
        50 55 60
Pro Ser Arg Met Gln Thr Ala Gly Glu Thr Ser Ser Arg His Gly Pro
65 70 75 80
Lys Lys Glu Leu Ser Cys Ser Phe Pro Val Phe Ser Thr Arg Asn His
                                85 90 95
Asn His Gly His Lys Gln Thr Ser Gln Phe His Val Pro Arg Phe Glu
                        100 105 110
Tyr Gln Glu Gly Gly Arg Glu Asn Pro Ala Val Val Ile Thr Lys Leu
                115 120 125
Thr Pro Phe His His Pro Lys Ile Ile Thr Ile Leu Phe Pro Ile Ser
        130 135 140
Asn Tyr Phe Ile Ile Phe Phe Phe Leu Thr Phe Asp Thr Lys Lys Gln
145 150 155 160
Tyr Pro Leu Leu Phe Pro Ile Leu Pro Ser Arg Phe Leu Pro Ile Ser
                                165 170 175
His Leu Ile Thr Gln Glu Ile Glu Lys Tyr Lys Thr Ser Ser His Phe
                        180 185 190
Ser Ser Pro Ala Ser Leu Phe Ala Ala Met Asn Lys Ala Glu Thr Ser
                195 200 205
Ser Met Ala Glu Ala Glu Ser Glu Asp Ser Glu Thr Thr Thr Pro Thr
        210 215 220
Thr His His Leu Thr Ile Pro Pro Gly Leu Thr Gln Pro Glu Phe Gln
225 230 235 240
Glu Leu Ala His Ser Ile Ser Glu Phe His Thr Tyr Gln Val Gly Pro
                                245 250 255
Gly Gln Cys Ser Ser Leu Leu Ala Gln Arg Val His Ala Pro Leu Pro
                        260 265 270
Thr Val Trp Ser Val Val Arg Arg Phe Asp Lys Pro Gln Thr Tyr Lys
                275 280 285
His Phe Ile Lys Ser Cys His Val Glu Asp Gly Phe Glu Met Arg Val
        290 295 300
Gly Cys Leu Arg Asp Val Asn Val Ile Ser Gly Leu Pro Ala Glu Thr
305 310 315 320
Ser Thr Glu Arg Leu Asp Ile Leu Asp Asp Glu Arg His Val Thr Gly
                                325 330 335
Phe Ser Ile Ile Gly Gly Glu His Arg Leu Arg Asn Tyr Arg Ser Val
                        340 345 350
Thr Thr Asn His Gly Gly Glu Ile Trp Thr Val Val Leu Glu Ser Tyr
                355 360 365
Val Val Asp Met Pro Glu Gly Asn Thr Glu Glu Asp Thr Arg Leu Phe
        370 375 380
Ala Asp Thr Val Val Lys Leu Asn Leu Gln Lys Leu Ala Ser Val Thr
385 390 395 400
Glu Val Ser Gln Ser Cys Asn Tyr Pro Cys Gln Phe His Ile Ile Glu
                                405 410 415
Asn Glu Asp Ile Gln Pro Glu Glu Met Asn Leu Gly Val Leu Thr Thr
                        420 425 430
Ser Ile Glu Glu Gln Arg Lys Lys Lys Arg Val Val Ala Met Lys Asp
                435 440 445
Gly Ser Thr Ser Ser
        450
```

| | |
|---|---|
| <210> | 18 |
| <211> | 195 |
| <212> | PRT |
| <213> | Vitis vinifera |
| <220> | |
| <223> | wine grape cultivar Pinot Noir hypothetical protein, clone |
| | ENTAV 115, locus tag VITISV_033963, GenBank Accession No. |
| | CAN64657.1, GI:147789129 |
| <220> | |
| <221> | VARIANT |
| <222> | (193)...(193) |
| <223> | Xaa = any amino acid |
| <400> | 18 |

```
Met Ala Glu Ala Glu Ser Glu Asp Ser Glu Thr Thr Thr Pro Thr Thr
  1 5 10 15
His His Leu Thr Ile Pro Pro Gly Leu Thr Gln Pro Glu Phe Gln Glu
                        20 25 30
Leu Ala His Ser Ile Ser Glu Phe His Thr Tyr Gln Val Gly Pro Gly
                35 40 45
Gln Cys Ser Ser Leu Leu Ala Gln Arg Val His Ala Pro Leu Pro Thr
        50 55 60
Val Trp Ser Val Val Arg Arg Phe Asp Lys Pro Gln Thr Tyr Lys His
65 70 75 80
Phe Ile Lys Ser Cys His Val Glu Asp Gly Phe Glu Met Arg Val Gly
                                85 90 95
Cys Leu Arg Asp Val Asn Val Ile Ser Gly Leu Pro Ala Glu Thr Ser
                        100 105 110
Thr Glu Arg Leu Asp Ile Leu Asp Asp Glu Arg His Val Thr Gly Phe
                115 120 125
Ser Ile Ile Gly Gly Glu His Arg Leu Arg Asn Tyr Arg Ser Val Thr
        130 135 140
Thr Val His Glu Tyr Gln Asn His Gly Gly Glu Ile Trp Thr Val Val
145 150 155 160
Leu Glu Ser Tyr Val Val Asp Met Pro Glu Gly Asn Thr Glu Glu Asp
                                165 170 175
Thr Arg Leu Phe Ala Asp Thr Val Val Lys Leu Asn Leu Ser Glu Ala
                        180 185 190
Xaa Arg Arg
              195
```

| | |
|---|---|
| <210> | 19 |
| <211> | 217 |
| <212> | PRT |
| <213> | Medicago truncatula |
| <220> | |
| <223> | barrel medic unknown protein, clone MTYFD_FE_FF_FG1G-N-24, |
| | GenBank Accession No. ACJ85026.1, GI:217073334 |
| <400> | 19 |

```
Met Glu Lys Ala Glu Ser Ser Thr Ala Ser Thr Ser Asp Gln Asp Ser
  1 5 10 15
Asp Glu Asn His Arg Thr Gln His His Leu Thr Leu Pro Ser Gly Leu
                        20 25 30
Arg Gln His Glu Phe Asp Ser Leu Ile Pro Phe Ile Asn Ser His His
                35 40 45
Thr Tyr Leu Ile Gly Pro Asn Gln Cys Ser Thr Leu Leu Ala Gln Arg
        50 55 60
Ile His Ala Pro Pro Gln Thr Val Trp Ser Val Val Arg Ser Phe Asp
65 70 75 80
Lys Pro Gln Ile Tyr Lys His Ile Ile Lys Ser Cys Ser Leu Lys Glu
                                85 90 95
Gly Phe Gln Met Lys Val Gly Cys Thr Arg Asp Val Asn Val Ile Ser
                        100 105 110
Gly Leu Pro Ala Ala Thr Ser Thr Glu Arg Leu Asp Val Leu Asp Asp
                115 120 125
Glu Arg Arg Val Thr Gly Phe Ser Ile Ile Gly Gly Glu His Arg Leu
        130 135 140
Lys Asn Tyr Arg Ser Val Thr Ser Val His Gly Phe Gly Asp Gly Asp
145 150 155 160
Asn Gly Gly Glu Ile Trp Thr Val Val Leu Glu Ser Tyr Val Val Asp
                                165 170 175
Val Pro Glu Gly Asn Thr Glu Glu Asp Thr Arg Leu Phe Ala Asp Thr
                        180 185 190
Val Val Lys Leu Asn Leu Gln Lys Leu Ala Ser Val Thr Glu Gly Lys
                195 200 205
Asn Arg Asp Gly Asp Gly Lys Ser His
        210 215
```

| | |
|---|---|
| <210> | 20 |
| <211> | 212 |
| <212> | PRT |
| <213> | Oryza sativa |
| <220> | |
| <223> | rice Japonica Group, cultivar Nipponbare, conserved |
| | hypothetical protein Os10g0573400, GenBank Accession No. |
| | NP_00106570.1, GI:115483600 |
| <400> | 20 |

```
Met Glu Gln Gln Glu Glu Val Pro Pro Pro Pro Ala Gly Leu Gly Leu
  1 5 10 15
Thr Ala Glu Glu Tyr Ala Gln Val Arg Ala Thr Val Glu Ala His His
                        20 25 30
Arg Tyr Ala Val Gly Pro Gly Gln Cys Ser Ser Leu Leu Ala Gln Arg
                35 40 45
Ile His Ala Pro Pro Ala Ala Val Trp Ala Val Val Arg Arg Phe Asp
        50 55 60
Cys Pro Gln Val Tyr Lys His Phe Ile Arg Ser Cys Val Leu Arg Pro
65 70 75 80
Asp Pro His His Asp Asp Asn Gly Asn Asp Leu Arg Pro Gly Arg Leu
                                85 90 95
Arg Glu Val Ser Val Ile Ser Gly Leu Pro Ala Ser Thr Ser Thr Glu
                        100 105 110
Arg Leu Asp Leu Leu Asp Asp Ala His Arg Val Phe Gly Phe Thr Ile
                115 120 125
Thr Gly Gly Glu His Arg Leu Arg Asn Tyr Arg Ser Val Thr Thr Val
        130 135 140
Ser Gln Leu Asp Glu Ile Cys Thr Leu Val Leu Glu Ser Tyr Ile Val
145 150 155 160
Asp Val Pro Asp Gly Asn Thr Glu Asp Asp Thr Arg Leu Phe Ala Asp
                                165 170 175
Thr Val Ile Arg Leu Asn Leu Gln Lys Leu Lys Ser Val Ser Glu Ala
                        180 185 190
Asn Ala Asn Ala Ala Ala Ala Ala Ala Ala Pro Pro Pro Pro Pro Pro
                195 200 205
Ala Ala Ala Glu
        210
```

| | |
|---|---|
| <210> | 21 |
| <211> | 212 |
| <212> | PRT |
| <213> | Zea mays |
| <220> | |
| <223> | maize cyclase/dehydrase family protein, clone 306819, |
| | GenBank Accession No. ACG40002.1, GI:195641068 |
| <400> | 21 |

```
Met Asp Gln Gln Gly Ala Gly Gly Asp Ala Glu Val Pro Ala Gly Leu
  1 5 10 15
Gly Leu Thr Ala Ala Glu Tyr Glu Gln Leu Arg Ser Thr Val Asp Ala
                        20 25 30
His His Arg Tyr Ala Val Gly Glu Gly Gln Cys Ser Ser Leu Leu Ala
                35 40 45
Gln Arg Ile His Ala Pro Pro Glu Ala Val Trp Ala Val Val Arg Arg
        50 55 60
Phe Asp Cys Pro Gln Val Tyr Lys His Phe Ile Arg Ser Cys Ala Leu
65 70 75 80
Arg Pro Asp Pro Glu Ala Gly Asp Ala Leu Cys Pro Gly Arg Leu Arg
                                85 90 95
Glu Val Ser Val Ile Ser Gly Leu Pro Ala Ser Thr Ser Thr Glu Arg
                        100 105 110
Leu Asp Leu Leu Asp Asp Ala Ala Arg Val Phe Gly Phe Ser Ile Thr
                115 120 125
Gly Gly Glu His Arg Leu Arg Asn Tyr Arg Ser Val Thr Thr Val Ser
        130 135 140
Glu Leu Ala Val Pro Ala Ile Cys Thr Val Val Leu Glu Ser Tyr Val
145 150 155 160
Val Asp Val Pro Asp Gly Asn Thr Glu Asp Asp Thr Arg Leu Phe Ala
                                165 170 175
Asp Thr Val Ile Arg Leu Asn Leu Gln Lys Leu Lys Ser Val Ala Glu
                        180 185 190
Ala Asn Ala Ala Glu Ala Ala Ala Thr Thr Asn Ser Val Leu Leu Pro
                195 200 205
Arg Pro Ala Glu
        210
```

| | |
|---|---|
| <210> | 22 |
| <211> | 212 |
| <212> | PRT |
| <213> | Zea mays |
| <220> | |
| <223> | maize cyclase/dehydrase family protein, clone 241996, |
| | GenBank Accession No. ACG34473.1, GI:195625286 |
| <220> | |
| <221> | VARIANT |
| <222> | (11)...(11) |
| <223> | Xaa = any amino acid |
| <400> | 22 |

```
Met Asp Gln Gln Gly Ala Gly Gly Asp Ala Xaa Val Pro Ala Gly Leu
  1 5 10 15
Gly Leu Thr Ala Ala Glu Tyr Glu Gln Leu Arg Ser Thr Val Asp Ala
                        20 25 30
His His Arg Tyr Ala Val Gly Glu Gly Gln Cys Ser Ser Leu Leu Ala
                35 40 45
Gln Arg Ile His Ala Pro Pro Glu Ala Val Trp Ala Val Val Arg Arg
        50 55 60
Phe Asp Cys Pro Gln Val Tyr Lys His Phe Ile Arg Ser Cys Ala Leu
65 70 75 80
Arg Pro Asp Pro Glu Ala Gly Asp Ala Leu Cys Pro Gly Arg Leu Arg
                                85 90 95
Glu Val Ser Val Ile Ser Gly Leu Pro Ala Ser Thr Ser Thr Glu Arg
                        100 105 110
Leu Asp Leu Leu Asp Asp Ala Ala Arg Val Phe Gly Phe Ser Ile Thr
                115 120 125
Gly Gly Glu His Arg Leu Arg Asn Tyr Arg Ser Val Thr Thr Val Ser
        130 135 140
Glu Leu Ala Asp Pro Ala Ile Cys Thr Val Val Leu Glu Ser Tyr Val
145 150 155 160
Val Asp Val Pro Asp Gly Asn Thr Glu Asp Asp Thr Arg Leu Phe Ala
                                165 170 175
Asp Thr Val Ile Arg Leu Asn Leu Gln Lys Leu Lys Ser Val Thr Glu
                        180 185 190
Ala Asn Ala Ala Glu Ala Ala Ala Thr Thr Asn Ser Val Leu Leu Pro
                195 200 205
Arg Pro Ala Glu
        210
```

| | |
|---|---|
| <210> | 23 |
| <211> | 233 |
| <212> | PRT |
| <213> | Vitis vinifera |
| <220> | |
| <223> | wine grape cultivar PN40024 unnamed protein product, locus |
| | tag GSVIVT00032173001, GenBank Accession No. CAO43790.1, |
| | GI:157339249 |
| <400> | 23 |

```
Met Asp Pro His His His His Gly Leu Thr Glu Glu Glu Phe Arg Ala
  1 5 10 15
Leu Glu Pro Ile Ile Gln Asn Tyr His Thr Phe Glu Pro Ser Pro Asn
                        20 25 30
Thr Cys Thr Ser Leu Ile Thr Gln Lys Ile Asp Ala Pro Ala Gln Val
                35 40 45
Val Trp Pro Phe Val Arg Ser Phe Glu Asn Pro Gln Lys Tyr Lys His
        50 55 60
Phe Ile Lys Asp Cys Thr Met Arg Gly Asp Gly Gly Val Gly Ser Ile
65 70 75 80
Arg Glu Val Thr Val Val Ser Gly Leu Pro Ala Ser Thr Ser Thr Glu
                                85 90 95
Arg Leu Glu Ile Leu Asp Asp Glu Lys His Ile Leu Ser Phe Arg Val
                        100 105 110
Val Gly Gly Glu His Arg Leu Asn Asn Tyr Arg Ser Val Thr Ser Val
                115 120 125
Asn Asp Phe Ser Lys Glu Gly Lys Asp Tyr Thr Ile Val Leu Glu Ser
        130 135 140
Tyr Ile Val Asp Ile Pro Glu Gly Asn Thr Gly Glu Asp Thr Lys Met
145 150 155 160
Phe Val Asp Thr Val Val Lys Leu Asn Leu Gln Lys Leu Ala Val Val
                                165 170 175
Ala Ile Thr Ser Leu His Glu Asn Glu Glu Ile Ala Asp Asn Glu Gly
                        180 185 190
Pro Ser Arg Glu Ile Ser Leu Gln Ser Glu Thr Glu Ser Ala Glu Arg
                195 200 205
Gly Asp Glu Arg Arg Asp Gly Asp Gly Pro Ser Lys Ala Cys Asn Arg
        210 215 220
Asn Glu Trp His Cys Thr Thr Lys Glu
225 230
```

| | |
|---|---|
| <210> | 24 |
| <211> | 207 |
| <212> | PRT |
| <213> | Oryza sativa |
| <220> | |
| <223> | rice Japonica Group, cultivar Nipponbare, Bet v I |
| | allergen-like protein, gene P0495C02.29, clone P0495C02, |
| | GenBank Accession No. BAD25659.1, GI:49388537 |
| <400> | 24 |

```
Met Glu Pro His Met Glu Arg Ala Leu Arg Glu Ala Val Ala Ser Glu
  1 5 10 15
Ala Glu Arg Arg Glu Leu Glu Gly Val Val Arg Ala His His Thr Phe
                        20 25 30
Pro Ala Ala Glu Arg Ala Ala Gly Pro Gly Arg Arg Pro Thr Cys Thr
                35 40 45
Ser Leu Val Ala Gln Arg Val Asp Ala Pro Leu Ala Ala Val Trp Pro
        50 55 60
Ile Val Arg Gly Phe Ala Asn Pro Gln Arg Tyr Lys His Phe Ile Lys
65 70 75 80
Ser Cys Glu Leu Ala Ala Gly Asp Gly Ala Thr Val Gly Ser Val Arg
                                85 90 95
Glu Val Ala Val Val Ser Gly Leu Pro Ala Ser Thr Ser Thr Glu Arg
                        100 105 110
Leu Glu Ile Leu Asp Asp Asp Arg His Val Leu Ser Phe Arg Val Val
                115 120 125
Gly Gly Asp His Arg Leu Arg Asn Tyr Arg Ser Val Thr Ser Val Thr
        130 135 140
Glu Phe Ser Ser Pro Ser Ser Pro Pro Arg Pro Tyr Cys Val Val Val
145 150 155 160
Glu Ser Tyr Val Val Asp Val Pro Glu Gly Asn Thr Glu Glu Asp Thr
                                165 170 175
Arg Met Phe Thr Asp Thr Val Val Lys Leu Asn Leu Gln Lys Leu Ala
                        180 185 190
Ala Val Ala Thr Ser Ser Ser Pro Pro Ala Ala Gly Asn His His
                195 200 205
```

| | |
|---|---|
| <210> | 25 |
| <211> | 210 |
| <212> | PRT |
| <213> | Oryza sativa |
| <220> | |
| <223> | rice Indica Group, cultivar 93-11, hypothetical protein |
| | OsI_06433, old locus tag OsI_006310, GLEAN gene, GenBank |
| | Accession No. EAY85077.1, GI:125538682 |
| <400> | 25 |

```
Met Glu Pro His Met Glu Arg Ala Leu Arg Glu Ala Val Ala Ser Glu
  1 5 10 15
Ala Glu Arg Arg Glu Leu Glu Gly Val Val Arg Ala His His Thr Phe
                        20 25 30
Pro Ala Ala Glu Arg Ala Ala Gly Pro Gly Arg Arg Pro Thr Cys Thr
                35 40 45
Ser Leu Val Ala Gln Arg Val Asp Ala Pro Leu Ala Ala Val Trp Pro
        50 55 60
Ile Val Arg Gly Phe Ala Asn Pro Gln Arg Tyr Lys His Phe Ile Lys
65 70 75 80
Ser Cys Glu Leu Ala Ala Gly Asp Gly Ala Thr Val Gly Ser Val Arg
                                85 90 95
Glu Val Ala Val Val Ser Gly Leu Pro Ala Ser Thr Ser Thr Glu Arg
                        100 105 110
Leu Glu Ile Leu Asp Asp Asp Arg His Val Leu Ser Phe Arg Val Val
                115 120 125
Gly Gly Asp His Arg Leu Arg Asn Tyr Arg Ser Val Thr Ser Val Thr
        130 135 140
Glu Phe Ser Ser Pro Ser Ser Pro Pro Ser Pro Pro Arg Pro Tyr Cys
145 150 155 160
Val Val Val Glu Ser Tyr Val Val Asp Val Pro Glu Gly Asn Thr Glu
                                165 170 175
Glu Asp Thr Arg Met Phe Thr Asp Thr Val Val Lys Leu Asn Leu Gln
                        180 185 190
Lys Leu Ala Ala Val Ala Thr Ser Ser Ser Pro Pro Ala Ala Gly Asn
                195 200 205
His His
```

| | |
|---|---|
| 210 | |
| <210> | 26 |
| <211> | 200 |
| <212> | PRT |
| <213> | Zea mays |
| <220> | |
| <223> | maize strain B73 unknown protein, clone ZM_BFb0151H07, |
| | GenBank Accession No. ACF82013.1, GI:194695858 |
| <400> | 26 |

```
Met Pro Tyr Thr Ala Pro Arg Pro Ser Pro Gln Gln His Ser Arg Val
  1 5 10 15
Leu Ser Gly Gly Gly Ala Lys Ala Ala Ser His Gly Ala Ser Cys Ala
                        20 25 30
Ala Val Pro Ala Glu Val Ala Arg His His Glu His Ala Ala Arg Ala
                35 40 45
Gly Gln Cys Cys Ser Ala Val Val Gln Ala Ile Ala Ala Pro Val Gly
        50 55 60
Ala Val Trp Ser Val Val Arg Arg Phe Asp Arg Pro Gln Ala Tyr Lys
65 70 75 80
His Phe Ile Arg Ser Cys Arg Leu Val Gly Gly Gly Asp Val Ala Val
                                85 90 95
Gly Ser Val Arg Glu Val Arg Val Val Ser Gly Leu Pro Ala Thr Ser
                        100 105 110
Ser Arg Glu Arg Leu Glu Ile Leu Asp Asp Glu Arg Arg Val Leu Ser
                115 120 125
Phe Arg Val Val Gly Gly Glu His Arg Leu Ala Asn Tyr Arg Ser Val
        130 135 140
Thr Thr Val His Glu Ala Gly Ala Gly Ala Gly Thr Gly Thr Val Val
145 150 155 160
Val Glu Ser Tyr Val Val Asp Val Pro His Gly Asn Thr Ala Asp Glu
                                165 170 175
Thr Arg Val Phe Val Asp Thr Ile Val Arg Cys Asn Leu Gln Ser Leu
                        180 185 190
Ala Arg Thr Ala Glu Arg Leu Ala
                195 200
```

| | |
|---|---|
| <210> | 27 |
| <211> | 215 |
| <212> | PRT |
| <213> | Vitis vinifera |
| <220> | |
| <223> | wine grape cultivar PN40024 unnamed protein product, locus |
| | tag GSVIVT00037390001, GenBank Accession No. CAO48777.1, |
| | GI:157355387 |
| <400> | 27 |

```
Met Pro Ser Asn Pro Pro Lys Ser Ser Leu Val Val His Arg Ile Asn
  1 5 10 15
Ser Pro Asn Ser Ile Thr Thr Ala Thr Thr Ala Ser Ala Ala Ala Asn
                        20 25 30
Asn His Asn Thr Ser Thr Met Pro Pro His Lys Gln Val Pro Asp Ala
                35 40 45
Val Ser Arg His His Thr His Val Val Gly Pro Asn Gln Cys Cys Ser
        50 55 60
Ala Val Val Gln Gln Ile Ala Ala Pro Val Ser Thr Val Trp Ser Val
65 70 75 80
Val Arg Arg Phe Asp Asn Pro Gln Ala Tyr Lys His Phe Val Lys Ser
                                85 90 95
Cys His Val Val Val Gly Asp Gly Asp Val Gly Thr Leu Arg Glu Val
                        100 105 110
His Val Ile Ser Gly Leu Pro Ala Ala Asn Ser Thr Glu Arg Leu Glu
                115 120 125
Ile Leu Asp Asp Glu Arg His Val Leu Ser Phe Ser Val Ile Gly Gly
        130 135 140
Asp His Arg Leu Ser Asn Tyr Arg Ser Val Thr Thr Leu His Pro Ser
145 150 155 160
Pro Ser Ser Thr Gly Thr Val Val Leu Glu Ser Tyr Val Val Asp Ile
                                165 170 175
Pro Pro Gly Asn Thr Lys Glu Asp Thr Cys Val Phe Val Asp Thr Ile
                        180 185 190
Val Arg Cys Asn Leu Gln Ser Leu Ala Gln Ile Ala Glu Asn Ala Ala
                195 200 205
Gly Cys Lys Arg Ser Ser Ser
        210 215
```

| | |
|---|---|
| <210> | 28 |
| <211> | 213 |
| <212> | PRT |
| <213> | Nicotiana tabacum |
| <220> | |
| <223> | tobacco hypothetical protein, gene c17, GenBank |
| | Accession No. CAI84653.1, GI:62867576 |
| <400> | 28 |

```
Met Pro Pro Ser Ser Pro Asp Ser Ser Val Leu Leu Gln Arg Ile Ser
  1 5 10 15
Ser Asn Thr Thr Pro Asp Phe Ala Cys Lys Gln Ser Gln Gln Leu Gln
                        20 25 30
Arg Arg Thr Met Pro Ile Pro Cys Thr Thr Gln Val Pro Asp Ser Val
                35 40 45
Val Arg Phe His Thr His Pro Val Gly Pro Asn Gln Cys Cys Ser Ala
        50 55 60
Val Ile Gln Arg Ile Ser Ala Pro Val Ser Thr Val Trp Ser Val Val
65 70 75 80
Arg Arg Phe Asp Asn Pro Gln Ala Tyr Lys His Phe Val Lys Ser Cys
                                85 90 95
His Val Ile Val Gly Asp Gly Asp Val Gly Thr Leu Arg Glu Val Arg
                        100 105 110
Val Ile Ser Gly Leu Pro Ala Ala Ser Ser Thr Glu Arg Leu Glu Ile
                115 120 125
Leu Asp Asp Glu Arg His Val Ile Ser Phe Ser Val Val Gly Gly Asp
        130 135 140
His Arg Leu Ala Asn Tyr Arg Ser Val Thr Thr Leu His Pro Glu Pro
145 150 155 160
Ser Gly Asp Gly Thr Thr Ile Val Val Glu Ser Tyr Val Val Asp Val
                                165 170 175
Pro Pro Gly Asn Thr Arg Asp Glu Thr Cys Val Phe Val Asp Thr Ile
                        180 185 190
Val Lys Cys Asn Leu Thr Ser Leu Ser Gln Ile Ala Val Asn Val Asn
                195 200 205
Arg Arg Lys Asp Ser
        210
```

| | |
|---|---|
| <210> | 29 |
| <211> | 208 |
| <212> | PRT |
| <213> | Oryza sativa |
| <220> | |
| <223> | rice Indica Group, cultivar 93-11, hypothetical protein |
| | OsI_04285, old locus tag OsI_004197, GLEAN gene, GenBank |
| | Accession No. EAY76350.1, GI:125528236 |
| <400> | 29 |

```
Met Pro Tyr Ala Ala Val Arg Pro Ser Pro Pro Pro Gln Leu Ser Arg
  1 5 10 15
Pro Ile Gly Ser Gly Ala Gly Gly Gly Lys Ala Cys Pro Ala Val Pro
                        20 25 30
Cys Glu Val Ala Arg Tyr His Glu His Ala Val Gly Ala Gly Gln Cys
                35 40 45
Cys Ser Thr Val Val Gln Ala Ile Ala Ala Pro Ala Asp Ala Val Trp
        50 55 60
Ser Val Val Arg Arg Phe Asp Arg Pro Gln Ala Tyr Lys Lys Phe Ile
65 70 75 80
Lys Ser Cys Arg Leu Val Asp Gly Asp Gly Gly Glu Val Gly Ser Val
                                85 90 95
Arg Glu Val Arg Val Val Ser Gly Leu Pro Ala Thr Ser Ser Arg Glu
                        100 105 110
Arg Leu Glu Val Leu Asp Asp Asp Arg Arg Val Leu Ser Phe Arg Ile
                115 120 125
Val Gly Gly Glu His Arg Leu Ala Asn Tyr Arg Ser Val Thr Thr Val
        130 135 140
His Glu Ala Ala Ala Pro Ala Met Ala Val Val Val Glu Ser Tyr Val
145 150 155 160
Val Asp Val Pro Pro Gly Asn Thr Trp Glu Glu Thr Arg Val Phe Val
                                165 170 175
Asp Thr Ile Val Arg Cys Asn Leu Gln Ser Leu Ala Arg Thr Val Glu
                        180 185 190
Arg Leu Ala Pro Glu Ala Pro Arg Ala Asn Gly Ser Ile Asp His Ala
                195 200 205
```

| | |
|---|---|
| <210> | 30 |
| <211> | 208 |
| <212> | PRT |
| <213> | Oryza sativa |
| <220> | |
| <223> | rice Japonica Group, cultivar Nipponbare, Bet v I |
| | allergen-like protein, gene B1088C09.11, clone B1088C09, |
| | GenBank Accession No. BAB68102.1, GI:15624049 |
| <400> | 30 |

```
Met Pro Tyr Ala Ala Val Arg Pro Ser Pro Pro Pro Gln Leu Ser Arg
  1 5 10 15
Pro Ile Gly Ser Gly Ala Gly Gly Gly Lys Ala Cys Pro Ala Val Pro
                        20 25 30
Cys Glu Val Ala Arg Tyr His Glu His Ala Val Gly Ala Gly Gln Cys
                35 40 45
Phe Ser Thr Val Val Gln Ala Ile Ala Ala Pro Ala Asp Ala Val Trp
        50 55 60
Ser Val Val Arg Arg Phe Asp Arg Pro Gln Ala Tyr Lys Lys Phe Ile
65 70 75 80
Lys Ser Cys Arg Leu Val Asp Gly Asp Gly Gly Glu Val Gly Ser Val
                                85 90 95
Arg Glu Val Arg Val Val Ser Gly Leu Pro Ala Thr Ser Ser Arg Glu
                        100 105 110
Arg Leu Glu Val Leu Asp Asp Asp Arg Arg Val Leu Ser Phe Arg Ile
                115 120 125
Val Gly Gly Glu His Arg Leu Ala Asn Tyr Arg Ser Val Thr Thr Val
        130 135 140
His Glu Ala Ala Ala Pro Ala Met Ala Val Val Val Glu Ser Tyr Val
145 150 155 160
Val Asp Val Pro Pro Gly Asn Thr Trp Glu Glu Thr Arg Val Phe Val
                                165 170 175
Asp Thr Ile Val Arg Cys Asn Leu Gln Ser Leu Ala Arg Thr Val Glu
                        180 185 190
Arg Leu Ala Pro Glu Ala Pro Arg Ala Asn Gly Ser Ile Asp His Ala
                195 200 205
```

| | |
|---|---|
| <210> | 31 |
| <211> | 213 |
| <212> | PRT |
| <213> | Picea sitchensis |
| <220> | |
| <223> | Sitka spruce cultivar FB3-425, unknown protein, |
| | clone WS0276_P02, GenBank Accession No. ABK22940.1, |
| | GI:116783434 |
| <400> | 31 |

```
Met Asp Ile Ile Ala Gly Phe Asp Gln Leu Ser Phe Arg Leu Ser Gly
  1 5 10 15
Ala Ser Lys Gln Ile Thr Lys Thr Gly Ala Val Gln Tyr Leu Lys Gly
                        20 25 30
Glu Glu Gly Tyr Gly Glu Trp Leu Lys Glu Val Met Gly Arg Tyr His
                35 40 45
Tyr His Ser His Asp Gly Ala Arg Glu Cys Arg Cys Ser Ser Val Val
        50 55 60
Val Gln Gln Val Glu Ala Pro Val Ser Val Val Trp Ser Leu Val Arg
65 70 75 80
Arg Phe Asp Gln Pro Gln Val Tyr Lys His Phe Val Ser Asn Cys Phe
                                85 90 95
Met Arg Gly Asp Leu Lys Val Gly Cys Leu Arg Glu Val Arg Val Val
                        100 105 110
Ser Gly Leu Pro Ala Ala Thr Ser Thr Glu Arg Leu Asp Ile Leu Asp
                115 120 125
Glu Glu Arg His Ile Leu Ser Phe Ser Ile Val Gly Gly Asp His Arg
        130 135 140
Leu Asn Asn Tyr Arg Ser Ile Thr Thr Leu His Glu Thr Leu Ile Asn
145 150 155 160
Gly Lys Pro Gly Thr Ile Val Ile Glu Ser Tyr Val Leu Asp Val Pro
                                165 170 175
His Gly Asn Thr Lys Glu Glu Thr Cys Leu Phe Val Asp Thr Ile Val
                        180 185 190
Lys Cys Asn Leu Gln Ser Leu Ala His Val Ser Asn His Leu Asn Ser
                195 200 205
Thr His Arg Cys Leu
        210
```

| | |
|---|---|
| <210> | 32 |
| <211> | 207 |
| <212> | PRT |
| <213> | Oryza sativa |
| <220> | |
| <223> | rice Japonica Group, cuitivar Nipponbare, hypothetical |
| | protein Os06g0562200, Bet v I allergen family protein, |
| | GenBank Accession No. NP_001057874.1, GI:115468550 |
| <400> | 32 |

```
Met Glu Ala His Val Glu Arg Ala Leu Arg Glu Gly Leu Thr Glu Glu
  1 5 10 15
Glu Arg Ala Ala Leu Glu Pro Ala Val Met Ala His His Thr Phe Pro
                        20 25 30
Pro Ser Thr Thr Thr Ala Thr Thr Ala Ala Ala Thr Cys Thr Ser Leu
                35 40 45
Val Thr Gln Arg Val Ala Ala Pro Val Arg Ala Val Trp Pro Ile Val
        50 55 60
Arg Ser Phe Gly Asn Pro Gln Arg Tyr Lys His Phe Val Arg Thr Cys
65 70 75 80
Ala Leu Ala Ala Gly Asp Gly Ala Ser Val Gly Ser Val Arg Glu Val
                                85 90 95
Thr Val Val Ser Gly Leu Pro Ala Ser Thr Ser Thr Glu Arg Leu Glu
                        100 105 110
Met Leu Asp Asp Asp Arg His Ile Ile Ser Phe Arg Val Val Gly Gly
                115 120 125
Gln His Arg Leu Arg Asn Tyr Arg Ser Val Thr Ser Val Thr Glu Phe
        130 135 140
Gln Pro Pro Ala Ala Gly Pro Gly Pro Ala Pro Pro Tyr Cys Val Val
145 150 155 160
Val Glu Ser Tyr Val Val Asp Val Pro Asp Gly Asn Thr Ala Glu Asp
                                165 170 175
Thr Arg Met Phe Thr Asp Thr Val Val Lys Leu Asn Leu Gln Met Leu
                        180 185 190
Ala Ala Val Ala Glu Asp Ser Ser Ser Ala Ser Arg Arg Arg Asp
                195 200 205
```

| | |
|---|---|
| <210> | 33 |
| <211> | 216 |
| <212> | PRT |
| <213> | Oryza sativa |
| <220> | |
| <223> | rice Japonica Group, cuitivar Nipponbare, hypothetical |
| | protein Os05g0473000, Streptomyces cyclase/dehydrase family |
| | protein, GenBank Accession No. NP_001055819.1, GI:115464439 |
| <400> | 33 |

```
Met Pro Tyr Thr Ala Pro Arg Pro Ser Pro Pro Gln His Ser Arg Ile
  1 5 10 15
Gly Gly Cys Gly Gly Gly Gly Val Leu Lys Ala Ala Gly Ala Ala Gly
                        20 25 30
His Ala Ala Ser Cys Val Ala Val Pro Ala Glu Val Ala Arg His His
                35 40 45
Glu His Ala Ala Gly Val Gly Gln Cys Cys Ser Ala Val Val Gln Ala
        50 55 60
Ile Ala Ala Pro Val Asp Ala Val Trp Ser Val Val Arg Arg Phe Asp
65 70 75 80
Arg Pro Gln Ala Tyr Lys His Phe Ile Arg Ser Cys Arg Leu Leu Asp
                                85 90 95
Gly Asp Gly Asp Gly Gly Ala Val Ala Val Gly Ser Val Arg Glu Val
                        100 105 110
Arg Val Val Ser Gly Leu Pro Ala Thr Ser Ser Arg Glu Arg Leu Glu
                115 120 125
Ile Leu Asp Asp Glu Arg Arg Val Leu Ser Phe Arg Val Val Gly Gly
        130 135 140
Glu His Arg Leu Ser Asn Tyr Arg Ser Val Thr Thr Val His Glu Thr
145 150 155 160
Ala Ala Gly Ala Ala Ala Ala Val Val Val Glu Ser Tyr Val Val Asp
                                165 170 175
Val Pro His Gly Asn Thr Ala Asp Glu Thr Arg Met Phe Val Asp Thr
                        180 185 190
Ile Val Arg Cys Asn Leu Gln Ser Leu Ala Arg Thr Ala Glu Gln Leu
                195 200 205
Ala Leu Ala Ala Pro Arg Ala Ala
        210 215
```

| | |
|---|---|
| <210> | 34 |
| <211> | 212 |
| <212> | PRT |
| <213> | Vitis vinifera |
| <220> | |
| <223> | wine grape cultivar PN40024 unnamed protein product, locus |
| | tag GSVIVT00029365001, GenBank Accession No. CAO41436.1, |
| | GI:157351249 |
| <400> | 34 |

```
Met Pro Ser Ser Leu Gln Leu His Arg Ile Asn Asn Ile Asp Pro Thr
  1 5 10 15
Thr Val Ala Val Ala Ala Thr Ala Ala Val Asn Cys His Lys Gln Ser
                        20 25 30
Arg Thr Pro Leu Arg Cys Ala Thr Pro Val Pro Asp Ala Val Ala Ser
                35 40 45
Tyr His Ala His Ala Val Gly Pro His Gln Cys Cys Ser Met Val Val
        50 55 60
Gln Thr Thr Ala Ala Ala Leu Pro Thr Val Trp Ser Val Val Arg Arg
65 70 75 80
Phe Asp Asn Pro Gln Ala Tyr Lys His Phe Leu Lys Ser Cys His Val
                                85 90 95
Ile Phe Gly Asp Gly Asp Ile Gly Thr Leu Arg Glu Val His Val Val
                        100 105 110
Ser Gly Leu Pro Ala Glu Ser Ser Thr Glu Arg Leu Glu Ile Leu Asp
                115 120 125
Asp Glu Arg His Val Leu Ser Phe Ser Val Val Gly Gly Asp His Arg
        130 135 140
Leu Cys Asn Tyr Arg Ser Val Thr Thr Leu His Pro Ser Pro Thr Gly
145 150 155 160
Thr Gly Thr Val Val Val Glu Ser Tyr Val Val Asp Ile Pro Pro Gly
                                165 170 175
Asn Thr Lys Glu Asp Thr Cys Val Phe Val Asp Thr Ile Val Lys Cys
                        180 185 190
Asn Leu Gln Ser Leu Ala Gln Met Ser Glu Lys Leu Thr Asn Asn Asn
                195 200 205
Arg Asn Ser Ser
        210
```

| | |
|---|---|
| <210> | 35 |
| <211> | 218 |
| <212> | PRT |
| <213> | Zea mays |
| <220> | |
| <223> | maize cyclase/dehydrase family protein, clone 1678999, |
| | GenBank Accession No. ACG30334.1, GI:195617008 |
| <400> | 35 |

```
Met Pro Cys Leu Gln Ala Ser Ser Pro Gly Ser Met Pro Tyr Gln His
  1 5 10 15
His Gly Arg Gly Val Gly Cys Ala Ala Glu Ala Gly Ala Ala Val Gly
                        20 25 30
Ala Ser Ala Gly Thr Gly Thr Arg Cys Gly Ala His Asp Gly Glu Val
                35 40 45
Pro Ala Glu Ala Ala Arg His His Glu His Ala Ala Pro Gly Pro Gly
        50 55 60
Arg Cys Cys Ser Ala Val Val Gln Arg Val Ala Ala Pro Ala Glu Ala
65 70 75 80
Val Trp Ser Val Val Arg Arg Phe Asp Gln Pro Gln Ala Tyr Lys Arg
                                85 90 95
Phe Val Arg Ser Cys Ala Leu Leu Ala Gly Asp Gly Gly Val Gly Thr
                        100 105 110
Leu Arg Glu Val Arg Val Val Ser Gly Leu Pro Ala Ala Ser Ser Arg
                115 120 125
Glu Arg Leu Glu Val Leu Asp Asp Glu Ser His Val Leu Ser Phe Arg
        130 135 140
Val Val Gly Gly Glu His Arg Leu Gln Asn Tyr Leu Ser Val Thr Thr
145 150 155 160
Val His Pro Ser Pro Ala Ala Pro Asp Ala Ala Thr Val Val Val Glu
                                165 170 175
Ser Tyr Val Val Asp Val Pro Pro Gly Asn Thr Pro Glu Asp Thr Arg
                        180 185 190
Val Phe Val Asp Thr Ile Val Lys Cys Asn Leu Gln Ser Leu Ala Thr
                195 200 205
Thr Ala Glu Lys Leu Ala Leu Ala Ala Val
        210 215
```

| | |
|---|---|
| <210> | 36 |
| <211> | 179 |
| <212> | PRT |
| <213> | Physcomitrella patens |
| <220> | |
| <223> | Physcomitrella patens subsp. patens bryophyte moss, ecotype |
| | Gransden 2004, hypothetical protein, predicted protein, |
| | locus tag PHYPADRAFT_222359, GenBank Accession No. |
| | XP_001778048.1, GI:168051209 |
| <400> | 36 |

```
Met Gln Thr Lys Gly Arg Gln Ala Asp Phe Gln Thr Leu Leu Glu Gly
  1 5 10 15
Gln Gln Asp Leu Ile Cys Arg Phe His Arg His Glu Leu Gln Pro His
                        20 25 30
Gln Cys Gly Ser Ile Leu Leu Gln Leu Ile Lys Ala Pro Val Glu Thr
                35 40 45
Val Trp Ser Val Ala Arg Ser Phe Asp Lys Pro Gln Val Tyr Lys Arg
        50 55 60
Phe Ile Gln Thr Cys Glu Ile Ile Glu Gly Asp Gly Gly Val Gly Ser
65 70 75 80
Ile Arg Glu Val Arg Leu Val Ser Ser Ile Pro Ala Thr Ser Ser Ile
                                85 90 95
Glu Arg Leu Glu Ile Leu Asp Asp Glu Glu His Ile Ile Ser Phe Arg
                        100 105 110
Val Leu Gly Gly Gly His Arg Leu Gln Asn Tyr Trp Ser Val Thr Ser
                115 120 125
Leu His Ser His Glu Ile Asp Gly Gln Met Gly Thr Leu Val Leu Glu
        130 135 140
Ser Tyr Val Val Asp Ile Pro Glu Gly Asn Thr Arg Glu Glu Thr His
145 150 155 160
Met Phe Val Asp Thr Val Val Arg Cys Asn Leu Lys Ala Leu Ala Gln
                                165 170 175
Val Ser Glu
```

| | |
|---|---|
| <210> | 37 |
| <211> | 229 |
| <212> | PRT |
| <213> | Oryza sativa |
| <220> | |
| <223> | rice Indica Group, cultivar 93-11, hypothetical protein |
| | OsI_11160, old locus tag OsI_010864, GLEAN gene, GenBank |
| | Accession No. EAY89631.1, GI:125543492 |
| <400> | 37 |

```
Met Pro Cys Ile Pro Ala Ser Ser Pro Gly Ile Pro His Gln His Gln
  1 5 10 15
His Gln His His Arg Ala Leu Ala Gly Val Gly Met Ala Val Gly Cys
                        20 25 30
Ala Ala Glu Ala Ala Val Ala Ala Ala Gly Val Ala Gly Thr Arg Cys
                35 40 45
Gly Ala His Asp Gly Glu Val Pro Met Glu Val Ala Arg His His Glu
        50 55 60
His Ala Glu Pro Gly Ser Gly Arg Cys Cys Ser Ala Val Val Gln His
65 70 75 80
Val Ala Ala Pro Ala Pro Ala Val Trp Ser Val Val Arg Arg Phe Asp
                                85 90 95
Gln Pro Gln Ala Tyr Lys Arg Phe Val Arg Ser Cys Ala Leu Leu Ala
                        100 105 110
Gly Asp Gly Gly Val Gly Thr Leu Arg Glu Val Arg Val Val Ser Gly
                115 120 125
Leu Pro Ala Ala Ser Ser Arg Glu Arg Leu Glu Ile Leu Asp Asp Glu
        130 135 140
Ser His Val Leu Ser Phe Arg Val Val Gly Gly Glu His Arg Leu Lys
145 150 155 160
Asn Tyr Leu Ser Val Thr Thr Val His Pro Ser Pro Ser Ala Pro Thr
                                165 170 175
Ala Ala Thr Val Val Val Glu Ser Tyr Val Val Asp Val Pro Pro Gly
                        180 185 190
Asn Thr Pro Glu Asp Thr Arg Val Phe Val Asp Thr Ile Val Lys Cys
                195 200 205
Asn Leu Gln Ser Leu Ala Lys Thr Ala Glu Lys Leu Ala Ala Gly Ala
        210 215 220
Arg Ala Ala Gly Ser
225
```

| | |
|---|---|
| <210> | 38 |
| <211> | 229 |
| <212> | PRT |
| <213> | Oryza sativa |
| <220> | |
| <223> | rice Japonica Group, cultivar Nipponbare, hypothetical |
| | protein Os03g0297600, Streptomyces cyclase/dehydrase family |
| | protein, GenBank Accession No. NP_001049838.1, GI:115452475 |
| <400> | 38 |

```
Met Pro Cys Ile Pro Ala Ser Ser Pro Gly Ile Pro His Gln His Gln
  1 5 10 15
His Gln His His Arg Ala Leu Ala Gly Val Gly Met Ala Val Gly Cys
                        20 25 30
Ala Ala Glu Ala Ala Val Ala Ala Ala Gly Val Ala Gly Thr Arg Cys
                35 40 45
Gly Ala His Asp Gly Glu Val Pro Met Glu Val Ala Arg His His Glu
        50 55 60
His Ala Glu Pro Gly Ser Gly Arg Cys Cys Ser Ala Val Val Gln His
65 70 75 80
Val Ala Ala Pro Ala Ala Ala Val Trp Ser Val Val Arg Arg Phe Asp
                                85 90 95
Gln Pro Gln Ala Tyr Lys Arg Phe Val Arg Ser Cys Ala Leu Leu Ala
                        100 105 110
Gly Asp Gly Gly Val Gly Thr Leu Arg Glu Val Arg Val Val Ser Gly
                115 120 125
Leu Pro Ala Ala Ser Ser Arg Glu Arg Leu Glu Ile Leu Asp Asp Glu
        130 135 140
Ser His Val Leu Ser Phe Arg Val Val Gly Gly Glu His Arg Leu Lys
145 150 155 160
Asn Tyr Leu Ser Val Thr Thr Val His Pro Ser Pro Ser Ala Pro Thr
                                165 170 175
Ala Ala Thr Val Val Val Glu Ser Tyr Val Val Asp Val Pro Pro Gly
                        180 185 190
Asn Thr Pro Glu Asp Thr Arg Val Phe Val Asp Thr Ile Val Lys Cys
                195 200 205
Asn Leu Gln Ser Leu Ala Lys Thr Ala Glu Lys Leu Ala Ala Gly Ala
        210 215 220
Arg Ala Ala Gly Ser
225
```

| | |
|---|---|
| <210> | 39 |
| <211> | 205 |
| <212> | PRT |
| <213> | Medicago truncatuia |
| <220> | |
| <223> | barrel medic unknown protein, clone MTYFP_FQ_FR_FS1G-H-19, |
| | GenBank Accession No. ACJ85898.1, GI:217075076 |
| <400> | 39 |

```
Met Pro Ser Pro Val Gln Phe Gln Arg Phe Asp Ser Asn Thr Ala Ile
  1 5 10 15
Thr Asn Gly Val Asn Cys Pro Lys Gln Ile Gln Ala Cys Arg Tyr Ala
                        20 25 30
Leu Ser Ser Leu Lys Pro Thr Val Ser Val Pro Glu Thr Val Val Asp
                35 40 45
His His Met His Val Val Gly Gln Asn Gln Cys Tyr Ser Val Val Ile
        50 55 60
Gln Thr Ile Asn Ala Ser Val Ser Thr Val Trp Ser Val Val Arg Arg
65 70 75 80
Phe Asp Tyr Pro Gln Gly Tyr Lys His Phe Val Lys Ser Cys Asn Val
                                85 90 95
Val Ala Ser Gly Asp Gly Ile Arg Val Gly Ala Leu Arg Glu Val Arg
                        100 105 110
Leu Val Ser Gly Leu Pro Ala Val Ser Ser Thr Glu Arg Leu Asp Ile
                115 120 125
Leu Asp Glu Glu Arg His Val Ile Ser Phe Ser Val Val Gly Gly Val
        130 135 140
His Arg Cys Arg Asn Tyr Arg Ser Val Thr Thr Leu His Gly Asp Gly
145 150 155 160
Asn Gly Gly Thr Val Val Ile Glu Ser Tyr Val Val Asp Val Pro Gln
                                165 170 175
Gly Asn Thr Lys Glu Glu Thr Cys Ser Phe Ala Asp Thr Ile Val Arg
                        180 185 190
Cys Asn Leu Gln Ser Leu Val Gln Ile Ala Glu Lys Leu
                195 200 205
```

| | |
|---|---|
| <210> | 40 |
| <211> | 212 |
| <212> | PRT |
| <213> | Zea mays |
| <220> | |
| <223> | maize AT-rich element binding factor 3, clone 1458362, |
| | GenBank Accession No. ACG26321.1, GI:195608982 |
| <400> | 40 |

```
Met Pro Phe Ala Ala Ser Arg Thr Ser Gln Gln Gln His Ser Arg Val
  1 5 10 15
Ala Thr Asn Gly Arg Ala Val Ala Val Cys Ala Gly His Ala Gly Val
                        20 25 30
Pro Asp Glu Val Ala Arg His His Glu His Ala Val Ala Ala Gly Gln
                35 40 45
Cys Cys Ala Ala Met Val Gln Ser Ile Ala Ala Pro Val Asp Ala Val
        50 55 60
Trp Ser Leu Val Arg Arg Phe Asp Gln Pro Gln Arg Tyr Lys Arg Phe
65 70 75 80
Ile Arg Ser Cys His Leu Val Asp Gly Asp Gly Ala Glu Val Gly Ser
                                85 90 95
Val Arg Glu Leu Leu Leu Val Ser Gly Leu Pro Ala Glu Ser Ser Arg
                        100 105 110
Glu Arg Leu Glu Ile Arg Asp Asp Glu Arg Arg Val Ile Ser Phe Arg
                115 120 125
Val Leu Gly Gly Asp His Arg Leu Ala Asn Tyr Arg Ser Val Thr Thr
        130 135 140
Val His Glu Ala Ala Pro Ser Gln Asp Gly Arg Pro Leu Thr Met Val
145 150 155 160
Val Glu Ser Tyr Val Val Asp Val Pro Pro Gly Asn Thr Val Glu Glu
                                165 170 175
Thr Arg Ile Phe Val Asp Thr Ile Val Arg Cys Asn Leu Gln Ser Leu
                        180 185 190
Glu Gly Thr Val Ile Arg Gln Leu Glu Ile Ala Ala Met Pro His Asp
                195 200 205
Asp Asn Gln Asn
        210
```

| | |
|---|---|
| <210> | 41 |
| <211> | 233 |
| <212> | PRT |
| <213> | Zea mays |
| <220> | |
| <223> | maize strain B73 unknown protein, clone ZM_BFb0105018, |
| | GenBank Accession No. ACF87013.1, GI:194705858 |
| <400> | 41 |

```
Met Arg Glu Arg Asn Ser Ser Ile Asp Gln Glu His Gln Arg Gly Ser
  1 5 10 15
Ser Ser Arg Ser Thr Met Pro Phe Ala Ala Ser Arg Thr Ser Gln Gln
                        20 25 30
Gln His Ser Arg Val Ala Thr Asn Gly Arg Ala Val Ala Val Cys Ala
                35 40 45
Gly His Ala Gly Val Pro Asp Glu Val Ala Arg His His Glu His Ala
        50 55 60
Val Ala Ala Gly Gln Cys Cys Ala Ala Met Val Gln Ser Ile Ala Ala
65 70 75 80
Pro Val Asp Ala Val Trp Ser Leu Val Arg Arg Phe Asp Gln Pro Gln
                                85 90 95
Arg Tyr Lys Arg Phe Ile Arg Ser Cys His Leu Val Asp Gly Asp Gly
                        100 105 110
Ala Glu Val Gly Ser Val Arg Glu Leu Leu Leu Val Ser Gly Leu Pro
                115 120 125
Ala Glu Ser Ser Arg Glu Arg Leu Glu Ile Arg Asp Asp Glu Arg Arg
        130 135 140
Val Ile Ser Phe Arg Val Leu Gly Gly Asp His Arg Leu Ala Asn Tyr
145 150 155 160
Arg Ser Val Thr Thr Val His Glu Ala Ala Pro Ser Gln Asp Gly Arg
                                165 170 175
Pro Leu Thr Met Val Val Glu Ser Tyr Val Val Asp Val Pro Pro Gly
                        180 185 190
Asn Thr Val Glu Glu Thr Arg Ile Phe Val Asp Thr Ile Val Arg Cys
                195 200 205
Asn Leu Gln Ser Leu Glu Gly Thr Val Ile Arg Gln Leu Glu Ile Ala
        210 215 220
Ala Met Pro His Asp Asp Asn Gln Asn
225 230
```

| | |
|---|---|
| <210> | 42 |
| <211> | 194 |
| <212> | PRT |
| <213> | Physcomitrella patens |
| <220> | |
| <223> | Physcomitrella patens subsp. patens bryophyte moss, ecotype |
| | Gransden 2004, hypothetical protein, predicted protein, |
| | locus tag PHYPADRAFT_209242, GenBank Accession No. |
| | XP_001762113.1, GI:168019160 |
| <400> | 42 |

```
Met Met Gln Glu Lys Gln Gly Arg Pro Asp Phe Gln Phe Leu Leu Glu
  1 5 10 15
Gly Gln Gln Asp Leu Ile Cys Arg Phe His Lys His Glu Leu Leu Pro
                        20 25 30
His Gln Cys Gly Ser Ile Leu Leu Gln Gln Ile Lys Ala Pro Val Gln
                35 40 45
Thr Val Trp Leu Ile Val Arg Arg Phe Asp Glu Pro Gln Val Tyr Lys
        50 55 60
Arg Phe Ile Gln Arg Cys Asp Ile Val Glu Gly Asp Gly Val Val Gly
65 70 75 80
Ser Ile Arg Glu Val Gln Leu Val Ser Ser Ile Pro Ala Thr Ser Ser
                                85 90 95
Ile Glu Arg Leu Glu Ile Leu Asp Asp Glu Glu His Ile Ile Ser Phe
                        100 105 110
Arg Val Leu Gly Gly Gly His Arg Leu Gln Asn Tyr Trp Ser Val Thr
                115 120 125
Ser Leu His Arg His Glu Ile Gln Gly Gln Met Gly Thr Leu Val Leu
        130 135 140
Glu Ser Tyr Val Val Asp Ile Pro Asp Gly Asn Thr Arg Glu Glu Thr
145 150 155 160
His Thr Phe Val Asp Thr Val Val Arg Cys Asn Leu Lys Ala Leu Ala
                                165 170 175
Gln Val Ser Glu Gln Lys His Leu Leu Asn Ser Asn Glu Lys Pro Ala
                        180 185 190
Ala Pro
```

| | |
|---|---|
| <210> | 43 |
| <211> | 191 |
| <212> | PRT |
| <213> | Vitis vinifera |
| <220> | |
| <223> | wine grape cuitivar PN40024 unnamed protein product, locus |
| | tag GSVIVT00035869001, GenBank Accession No. CAO48052.1, |
| | GI:157354734 |
| <400> | 43 |

```
Met Lys Val Tyr Ser Pro Ser Gln Ile Leu Ala Glu Arg Gly Pro Arg
  1 5 10 15
Ala Gln Ala Met Gly Asn Leu Tyr His Thr His His Leu Leu Pro Asn
                        20 25 30
Gln Cys Ser Ser Leu Val Val Gln Thr Thr Asp Ala Pro Leu Pro Gln
                35 40 45
Val Trp Ser Met Val Arg Arg Phe Asp Arg Pro Gln Ser Tyr Lys Arg
        50 55 60
Phe Val Arg Gly Cys Thr Leu Arg Arg Gly Lys Gly Gly Val Gly Ser
65 70 75 80
Val Arg Glu Val Asn Ile Val Ser Gly Leu Pro Ala Glu Ile Ser Leu
                                85 90 95
Glu Arg Leu Asp Lys Leu Asp Asp Asp Leu His Val Met Arg Phe Thr
                        100 105 110
Val Ile Gly Gly Asp His Arg Leu Ala Asn Tyr His Ser Thr Leu Thr
                115 120 125
Leu His Glu Asp Glu Glu Asp Gly Val Arg Lys Thr Val Val Met Glu
        130 135 140
Ser Tyr Val Val Asp Val Pro Gly Gly Asn Ser Ala Gly Glu Thr Cys
145 150 155 160
Tyr Phe Ala Asn Thr Ile Ile Gly Phe Asn Leu Lys Ala Leu Ala Ala
                                165 170 175
Val Thr Glu Thr Met Ala Leu Lys Ala Asn Ile Pro Ser Gly Phe
                        180 185 190
```

| | |
|---|---|
| <210> | 44 |
| <211> | 217 |
| <212> | PRT |
| <213> | Physcomitrella patens |
| <220> | |
| <223> | Physcomitrella patens subsp. patens bryophyte moss, ecotype |
| | Gransden 2004, hypothetical protein, predicted protein, |
| | locus tag PHYPADRAFT_132509, GenBank Accession No. |
| | XP_001767821.1, GI:168030621 |
| <400> | 44 |

```
Met Gln Gln Val Lys Gly Arg Gln Asp Phe Gln Arg Leu Leu Glu Ala
  1 5 10 15
Gln Gln Asp Leu Ile Cys Arg Tyr His Thr His Glu Leu Lys Ala His
                        20 25 30
Gln Cys Gly Ser Ile Leu Leu Gln Gln Ile Lys Val Pro Leu Pro Ile
                35 40 45
Val Trp Ala Ile Val Arg Ser Phe Asp Lys Pro Gln Val Tyr Lys Arg
        50 55 60
Phe Ile Gln Thr Cys Lys Ile Thr Glu Gly Asp Gly Gly Val Gly Ser
65 70 75 80
Ile Arg Glu Val His Leu Val Ser Ser Val Pro Ala Thr Cys Ser Ile
                                85 90 95
Glu Arg Leu Glu Ile Leu Asp Asp Glu Lys His Ile Ile Ser Phe Arg
                        100 105 110
Val Leu Gly Gly Gly His Arg Leu Gln Asn Tyr Ser Ser Val Ser Ser
                115 120 125
Leu His Glu Leu Glu Val Glu Gly His Pro Cys Thr Leu Val Leu Glu
        130 135 140
Ser Tyr Met Val Asp Ile Pro Asp Gly Asn Thr Arg Glu Glu Thr His
145 150 155 160
Met Phe Val Asp Thr Val Val Arg Cys Asn Leu Lys Ser Leu Ala Gln
                                165 170 175
Ile Ser Glu Gln Gln Tyr Asn Lys Asp Cys Leu Gln Gln Lys Gln His
                        180 185 190
Asp Gln Gln Gln Met Tyr Gln Gln Arg His Pro Pro Leu Pro Pro Ile
                195 200 205
Pro Ile Thr Asp Lys Asn Met Glu Arg
        210 215
```

| | |
|---|---|
| <210> | 45 |
| <211> | 195 |
| <212> | PRT |
| <213> | Physcomitrella patens |
| <220> | |
| <223> | Physcomitrella patens subsp. patens bryophyte moss, ecotype |
| | Gransden 2004, hypothetical protein, predicted protein, |
| | locus tag PHYPADRAFT_213389, GenBank Accession No. |
| | XP_001767012.1, GI:168028995 |
| <400> | 45 |

```
Met Arg Phe Asp Ile Gly His Asn Asp Val Arg Gly Phe Phe Thr Cys
  1 5 10 15
Glu Glu Glu His Ala Tyr Ala Leu His Ser Gln Thr Val Glu Leu Asn
                        20 25 30
Gln Cys Gly Ser Ile Leu Met Gln Gln Ile His Ala Pro Ile Glu Val
                35 40 45
Val Trp Ser Ile Val Arg Ser Phe Gly Ser Pro Gln Ile Tyr Lys Lys
        50 55 60
Phe Ile Gln Ala Cys Ile Leu Thr Val Gly Asp Gly Gly Val Gly Ser
65 70 75 80
Ile Arg Glu Val Phe Leu Val Ser Gly Val Pro Ala Thr Ser Ser Ile
                                85 90 95
Glu Arg Leu Glu Ile Leu Asp Asp Glu Lys His Val Phe Ser Phe Arg
                        100 105 110
Val Leu Lys Gly Gly His Arg Leu Gln Asn Tyr Arg Ser Val Thr Thr
                115 120 125
Leu His Glu Gln Glu Val Asn Gly Arg Gln Thr Thr Thr Val Leu Glu
        130 135 140
Ser Tyr Val Val Asp Val Pro Asp Gly Asn Thr Arg Glu Glu Thr His
145 150 155 160
Met Phe Ala Asp Thr Val Val Met Cys Asn Leu Lys Ser Leu Ala Gln
                                165 170 175
Val Ala Glu Trp Arg Ala Met Gln Gly Ile Thr Gln Gln Leu Ser Thr
                        180 185 190
Ser Ser Leu
                195
```

| | |
|---|---|
| <210> | 46 |
| <211> | 172 |
| <212> | PRT |
| <213> | Vitis vinifera |
| <220> | |
| <223> | wine grape cultivar Pinot Noir hypothetical protein, clone |
| | ENTAV 115, locus tag VITISV_004947, GenBank Accession No. |
| | CAN72620.1, GI:147840019 |
| <400> | 46 |

```
Met Gly Asn Leu Tyr His Thr His His Leu Leu Pro Asn Gln Cys Ser
  1 5 10 15
Ser Leu Val Val Gln Thr Thr Asp Ala Pro Leu Pro Gln Val Trp Ser
                        20 25 30
Met Val Arg Arg Phe Asp Arg Pro Gln Ser Tyr Lys Arg Phe Val Arg
                35 40 45
Gly Cys Thr Leu Arg Arg Gly Lys Gly Gly Val Gly Ser Val Arg Glu
        50 55 60
Val Asn Ile Val Ser Gly Leu Pro Ala Glu Ile Ser Leu Glu Arg Leu
65 70 75 80
Asp Lys Leu Asp Asp Asp Leu His Val Met Arg Phe Thr Val Ile Gly
                                85 90 95
Gly Asp His Arg Leu Ala Asn Tyr His Ser Thr Leu Thr Leu His Glu
                        100 105 110
Asp Glu Glu Asp Gly Val Arg Lys Thr Val Val Met Glu Ser Tyr Val
                115 120 125
Val Asp Val Pro Gly Gly Asn Ser Ala Gly Glu Thr Cys Tyr Phe Ala
        130 135 140
Asn Thr Ile Ile Gly Phe Asn Leu Lys Ala Leu Ala Ala Val Thr Glu
145 150 155 160
Thr Met Ala Leu Lys Ala Asn Ile Pro Ser Gly Phe
                                165 170
```

| | |
|---|---|
| <210> | 47 |
| <211> | 196 |
| <212> | PRT |
| <213> | Picea sitchensis |
| <220> | |
| <223> | Sitka spruce cultivar FB3-425, unknown protein, |
| | clone WS0281_124, GenBank Accession No. ABK23752.1, |
| | GI:116785512 |
| <400> | 47 |

```
Met Glu Asp Leu Ser Ser Trp Arg Glu Gly Arg Ala Met Trp Leu Gly
  1 5 10 15
Asn Pro Pro Ser Glu Ser Glu Leu Val Cys Arg His His Arg His Glu
                        20 25 30
Leu Gln Gly Asn Gln Cys Ser Ser Phe Leu Val Lys His Ile Arg Ala
                35 40 45
Pro Val His Leu Val Trp Ser Ile Val Arg Thr Phe Asp Gln Pro Gln
        50 55 60
Lys Tyr Lys Pro Phe Val His Ser Cys Ser Val Arg Gly Gly Ile Thr
65 70 75 80
Val Gly Ser Ile Arg Asn Val Asn Val Lys Ser Gly Leu Pro Ala Thr
                                85 90 95
Ala Ser Glu Glu Arg Leu Glu Ile Leu Asp Asp Asn Glu His Val Phe
                        100 105 110
Ser Ile Lys Ile Leu Gly Gly Asp His Arg Leu Gln Asn Tyr Ser Ser
                115 120 125
Ile Ile Thr Val His Pro Glu Ile Ile Asp Gly Arg Pro Gly Thr Leu
        130 135 140
Val Ile Glu Ser Tyr Val Val Asp Val Pro Glu Gly Asn Thr Arg Glu
145 150 155 160
Glu Thr Arg Phe Phe Val Glu Ala Leu Val Lys Cys Asn Leu Lys Ser
                                165 170 175
Leu Ala Asp Val Ser Glu Arg Leu Ala Ser Gln His His Thr Glu Leu
                        180 185 190
Leu Glu Arg Thr
                195
```

| | |
|---|---|
| <210> | 48 |
| <211> | 185 |
| <212> | PRT |
| <213> | Solanum tuberosum |
| <220> | |
| <223> | potato cultivar Kuras, CAPIP1-like protein, clone 153D02, |
| | similar to Capsicum annuum CAPIP1, GenBank Accession No. |
| | ABB29920.1, GI:78191398 |
| <400> | 48 |

```
Met Asn Ala Asn Gly Phe Cys Gly Val Glu Lys Glu Tyr Ile Arg Lys
  1 5 10 15
His His Leu His Glu Pro Lys Glu Asn Gln Cys Ser Ser Phe Leu Val
                        20 25 30
Lys His Ile Arg Ala Pro Val His Leu Val Trp Ser Leu Val Arg Arg
                35 40 45
Phe Asp Gln Pro Gln Lys Tyr Lys Pro Phe Ile Ser Arg Cys Ile Val
        50 55 60
Gln Gly Asp Leu Glu Ile Gly Ser Leu Arg Glu Val Asp Val Lys Ser
65 70 75 80
Gly Leu Pro Ala Thr Thr Ser Thr Glu Arg Leu Glu Leu Leu Asp Asp
                                85 90 95
Glu Glu His Ile Leu Ser Val Arg Ile Val Gly Gly Asp His Arg Leu
                        100 105 110
Arg Asn Tyr Ser Ser Val Ile Ser Val His Pro Glu Val Ile Asp Gly
                115 120 125
Arg Pro Gly Thr Val Val Leu Glu Ser Phe Val Val Asp Val Pro Glu
        130 135 140
Gly Asn Thr Lys Asp Glu Thr Cys Tyr Phe Val Glu Ala Leu Ile Asn
145 150 155 160
Cys Asn Leu Lys Ser Leu Ala Asp Ile Ser Glu Arg Val Ala Val Gln
                                165 170 175
Asp Arg Thr Glu Pro Ile Asp Gln Val
                        180 185
```

| | |
|---|---|
| <210> | 49 |
| <211> | 190 |
| <212> | PRT |
| <213> | Medicago truncatula |
| <220> | |
| <223> | barrel medic unknown protein, clone MTYFP_FQ_FR_FS1G-E-17, |
| | GenBank Accession No. ACJ85952.1, GI:217075184 |
| <400> | 49 |

```
Met Asn Asn Gly Cys Glu Gln Gln Gln Tyr Ser Val Ile Glu Thr Gln
  1 5 10 15
Tyr Ile Arg Arg His His Lys His Asp Leu Arg Asp Asn Gln Cys Ser
                        20 25 30
Ser Ala Leu Val Lys His Ile Lys Ala Pro Val His Leu Val Trp Ser
                35 40 45
Leu Val Arg Arg Phe Asp Gln Pro Gln Lys Tyr Lys Pro Phe Ile Ser
        50 55 60
Arg Cys Ile Met Gln Gly Asp Leu Ser Ile Gly Ser Val Arg Glu Val
65 70 75 80
Asn Val Lys Ser Gly Leu Pro Ala Thr Thr Ser Thr Glu Arg Leu Glu
                                85 90 95
Gln Leu Asp Asp Glu Glu His Ile Leu Gly Ile Arg Ile Val Gly Gly
                        100 105 110
Asp His Arg Leu Arg Asn Tyr Ser Ser Ile Ile Thr Val His Pro Gly
                115 120 125
Val Ile Asp Gly Arg Pro Gly Thr Met Val Ile Glu Ser Phe Val Val
        130 135 140
Asp Val Pro Glu Gly Asn Thr Lys Asp Glu Thr Cys Tyr Phe Val Glu
145 150 155 160
Ala Leu Ile Arg Tyr Asn Leu Ser Ser Leu Ala Asp Val Ser Glu Arg
                                165 170 175
Met Ala Val Gln Gly Arg Thr Asp Pro Ile Asn Ile Asn Pro
                        180 185 190
```

| | |
|---|---|
| <210> | 50 |
| <211> | 185 |
| <212> | PRT |
| <213> | Vitis vinifera |
| <220> | |
| <223> | wine grape cultivar PN40024 unnamed protein product, locus |
| | tag GSVIVT00002440001, GenBank Accession No. CAO65816.1, |
| | GI:157358179 |
| <400> | 50 |

```
Met Ser Gly Tyr Gly Cys Ile Lys Met Glu Asp Glu Tyr Ile Arg Arg
  1 5 10 15
His His Arg His Glu Ile Arg Asp Asn Gln Cys Ser Ser Ser Leu Val
                        20 25 30
Lys His Ile Lys Ala Pro Val His Leu Val Trp Ser Leu Val Arg Ser
                35 40 45
Phe Asp Gln Pro Gln Lys Tyr Lys Pro Phe Val Ser Arg Cys Ile Val
        50 55 60
Gln Gly Asp Leu Glu Ile Gly Ser Val Arg Glu Val Asn Val Lys Ser
65 70 75 80
Gly Leu Pro Ala Thr Thr Ser Thr Glu Arg Leu Glu Leu Leu Asp Asp
                                85 90 95
Glu Glu His Ile Phe Gly Met Arg Ile Val Gly Gly Asp His Arg Leu
                        100 105 110
Lys Asn Tyr Ser Ser Ile Val Thr Val His Pro Glu Ile Ile Asp Gly
                115 120 125
Arg Pro Gly Thr Leu Val Ile Glu Ser Phe Val Val Asp Val Pro Asp
        130 135 140
Gly Asn Thr Lys Asp Glu Thr Cys Tyr Phe Val Glu Ala Leu Ile Lys
145 150 155 160
Cys Asn Leu Lys Ser Leu Ala Asp Val Ser Glu Arg Leu Ala Ile Gln
                                165 170 175
Asp Arg Thr Glu Pro Ile Asp Arg Met
                        180 185
```

| | |
|---|---|
| <210> | 51 |
| <211> | 185 |
| <212> | PRT |
| <213> | Vitis vinifera |
| <220> | |
| <223> | wine grape cultivar PN40024 unnamed protein product, locus |
| | tag GSVIVT00006507001, GenBank Accession No. CAO69376.1, |
| | GI:157360187 |
| <400> | 51 |

```
Met Asn Gly Asn Gly Leu Ser Ser Met Glu Ser Glu Tyr Ile Arg Arg
  1 5 10 15
His His Arg His Glu Pro Ala Glu Asn Gln Cys Ser Ser Ala Leu Val
                        20 25 30
Lys His Ile Lys Ala Pro Val Pro Leu Val Trp Ser Leu Val Arg Arg
                35 40 45
Phe Asp Gln Pro Gln Lys Tyr Lys Pro Phe Ile Ser Arg Cys Val Val
        50 55 60
Gln Gly Asn Leu Glu Ile Gly Ser Leu Arg Glu Val Asp Val Lys Ser
65 70 75 80
Gly Leu Pro Ala Thr Thr Ser Thr Glu Arg Leu Glu Leu Leu Asp Asp
                                85 90 95
Asp Glu His Ile Leu Ser Met Arg Ile Ile Gly Gly Asp His Arg Leu
                        100 105 110
Arg Asn Tyr Ser Ser Ile Ile Ser Leu His Pro Glu Ile Ile Asp Gly
                115 120 125
Arg Pro Gly Thr Met Val Ile Glu Ser Tyr Val Val Asp Val Pro Glu
        130 135 140
Gly Asn Thr Lys Asp Glu Thr Cys Tyr Phe Val Glu Ala Leu Ile Lys
145 150 155 160
Cys Asn Leu Lys Ser Leu Ala Asp Val Ser Glu Arg Leu Ala Val Gln
                                165 170 175
Asp Arg Thr Glu Pro Ile Asp Arg Met
                        180 185
```

| | |
|---|---|
| <210> | 52 |
| <211> | 208 |
| <212> | PRT |
| <213> | Oryza sativa |
| <220> | |
| <223> | rice Japonica Group, cultivar Nipponbare, hypothetical |
| | protein OsJ_21703, old locus tag OsJ_020847, GLEAN gene, |
| | GenBank Accession No. EAZ37364.1, GI:125597584 |
| <400> | 52 |

```
Met Glu Ala His Val Glu Arg Ala Leu Arg Glu Gly Leu Thr Glu Glu
  1 5 10 15
Glu Arg Ala Ala Leu Glu Pro Ala Val Met Ala His His Thr Phe Pro
                        20 25 30
Pro Ser Thr Thr Thr Ala Thr Thr Ala Ala Ala Thr Cys Thr Ser Leu
                35 40 45
Val Thr Gln Arg Val Ala Ala Pro Val Arg Ala Val Trp Pro Ile Val
        50 55 60
Arg Ser Phe Gly Asn Pro Gln Arg Tyr Lys His Phe Val Arg Thr Cys
65 70 75 80
Ala Leu Ala Ala Gly Asn Gly Pro Ser Phe Gly Ser Val Arg Glu Val
                                85 90 95
Thr Val Val Ser Gly Pro Ser Arg Leu Pro Pro Gly Thr Glu Arg Leu
                        100 105 110
Glu Met Leu Asp Asp Asp Arg His Ile Ile Ser Phe Arg Val Val Gly
                115 120 125
Gly Gln His Arg Leu Arg Asn Tyr Arg Ser Val Thr Ser Val Thr Glu
        130 135 140
Phe Gln Pro Pro Ala Ala Gly Pro Gly Pro Ala Pro Pro Tyr Cys Val
145 150 155 160
Val Val Glu Ser Tyr Val Val Asp Val Pro Asp Gly Asn Thr Ala Glu
                                165 170 175
Asp Thr Arg Met Phe Thr Asp Thr Val Val Lys Leu Asn Leu Gln Met
                        180 185 190
Leu Ala Ala Val Ala Glu Asp Ser Ser Ser Ala Ser Arg Arg Arg Asp
                195 200 205
```

| | |
|---|---|
| <210> | 53 |
| <211> | 186 |
| <212> | PRT |
| <213> | Capsicum annuum |
| <220> | |
| <223> | pepper cultivar hanbyul, CAPIP1 protein, GenBank |
| | Accession No. AAT35532.1, GI:47558817 |
| <400> | 53 |

```
Met Met Asn Ala Asn Gly Phe Ser Gly Val Glu Lys Glu Tyr Ile Arg
  1 5 10 15
Lys His His Leu His Gln Pro Lys Glu Asn Gln Cys Ser Ser Phe Leu
                        20 25 30
Val Lys His Ile Arg Ala Pro Val His Leu Val Trp Ser Leu Val Arg
                35 40 45
Arg Phe Asp Gln Pro Gln Lys Tyr Lys Pro Phe Val Ser Arg Cys Ile
        50 55 60
Ala Gln Gly Asp Leu Glu Ile Gly Ser Leu Arg Glu Val Asp Val Lys
65 70 75 80
Ser Gly Leu Pro Ala Thr Thr Ser Thr Glu Arg Leu Glu Leu Leu Asp
                                85 90 95
Asp Glu Glu His Ile Leu Ser Phe Arg Ile Ile Gly Gly Asp His Arg
                        100 105 110
Leu Arg Asn Tyr Ser Ser Ile Ile Ser Leu His Pro Glu Val Ile Asp
                115 120 125
Gly Arg Pro Gly Thr Leu Val Ile Glu Ser Phe Val Val Asp Val Pro
        130 135 140
Gln Gly Asn Thr Lys Asp Glu Thr Cys Tyr Phe Val Glu Ala Leu Ile
145 150 155 160
Asn Cys Asn Leu Lys Ser Leu Ala Asp Val Ser Glu Arg Leu Ala Val
                                165 170 175
Gln Asp Arg Thr Glu Pro Ile Asp Gln Val
                        180 185
```

| | |
|---|---|
| <210> | 54 |
| <211> | 186 |
| <212> | PRT |
| <213> | Populus trichocarpa |
| <220> | |
| <223> | California poplar (Western balsam poplar, black cottonwood) |
| | cultivar 383-2499 (Nisqually-1), unknown protein, clone |
| | PX0011_1113, GenBank Accession No. ABK92491.1, GI:118481075 |
| <400> | 54 |

```
Met Asn Gly Ser Asp Ala Tyr Ser Ala Thr Glu Ala Gln Tyr Val Arg
  1 5 10 15
Arg His His Lys His Glu Pro Arg Glu Asn Gln Cys Thr Ser Ala Leu
                        20 25 30
Val Lys His Ile Lys Ala Pro Ala His Leu Val Trp Ser Leu Val Arg
                35 40 45
Arg Phe Asp Gln Pro Gln Arg Tyr Lys Pro Phe Val Ser Arg Cys Val
        50 55 60
Met Asn Gly Glu Leu Gly Ile Gly Ser Val Arg Glu Val Asn Val Lys
65 70 75 80
Ser Gly Leu Pro Ala Thr Thr Ser Thr Glu Arg Leu Glu Leu Leu Asp
                                85 90 95
Asp Glu Glu His Ile Leu Gly Val Gln Ile Val Gly Gly Asp His Arg
                        100 105 110
Leu Lys Asn Tyr Ser Ser Ile Met Thr Val His Pro Glu Phe Ile Asp
                115 120 125
Gly Arg Pro Gly Thr Leu Val Ile Glu Ser Phe Ile Val Asp Val Pro
        130 135 140
Asp Gly Asn Thr Lys Asp Glu Thr Cys Tyr Phe Val Glu Ala Leu Ile
145 150 155 160
Arg Cys Asn Leu Lys Ser Leu Ala Asp Val Ser Glu Arg Met Ala Val
                                165 170 175
Gln Asp Arg Val Glu Pro Val Asn Gln Phe
                        180 185
```

| | |
|---|---|
| <210> | 55 |
| <211> | 185 |
| <212> | PRT |
| <213> | Capsicum annuum |
| <220> | |
| <223> | pepper cultivar hanbyul, PIP1 protein, GenBank |
| | Accession No. ABF72432.1, GI:104304209 |
| <400> | 55 |

```
Met Asn Ala Asn Gly Phe Ser Gly Val Glu Lys Glu Tyr Ile Arg Lys
  1 5 10 15
His His Leu His Gln Pro Lys Glu Asn Gln Cys Ser Ser Phe Leu Val
                        20 25 30
Lys His Ile Arg Ala Pro Val His Leu Val Trp Ser Leu Val Arg Arg
                35 40 45
Phe Asp Gln Pro Gln Lys Tyr Lys Pro Phe Val Ser Arg Cys Ile Ala
        50 55 60
Gln Gly Asp Leu Glu Ile Gly Ser Leu Arg Glu Val Asp Val Lys Ser
65 70 75 80
Gly Leu Pro Ala Thr Thr Ser Thr Glu Arg Leu Glu Leu Leu Asp Asp
                                85 90 95
Glu Glu His Ile Leu Ser Phe Arg Ile Ile Gly Gly Asp His Arg Leu
                        100 105 110
Arg Asn Tyr Ser Ser Ile Ile Ser Leu His Pro Glu Val Ile Asp Gly
                115 120 125
Arg Pro Gly Thr Leu Val Ile Glu Ser Phe Val Val Asp Val Pro Gln
        130 135 140
Gly Asn Thr Lys Asp Glu Thr Cys Tyr Phe Val Glu Ala Leu Ile Asn
145 150 155 160
Cys Asn Leu Lys Ser Leu Ala Asp Val Ser Glu Arg Leu Ala Val Gln
                                165 170 175
Asp Arg Thr Glu Pro Ile Asp Gln Val
                        180 185
```

| | |
|---|---|
| <210> | 56 |
| <211> | 186 |
| <212> | PRT |
| <213> | Populus trichocarpa x Populus deltoides |
| <220> | |
| <223> | California poplar (Western balsam poplar, black cottonwood) |
| | x Eastern cottonwood, cultivar H11-11, unknown protein, |
| | clone WS0133_104, GenBank Accession No. ABK96505.1, |
| | GI:118489403 |
| <400> | 56 |

```
Met Asn Gly Ser Asp Ala Tyr Ser Ala Thr Glu Ala Gln Tyr Val Arg
  1 5 10 15
Arg His His Lys His Glu Pro Arg Glu Asn Gln Cys Thr Ser Ala Leu
                        20 25 30
Val Lys His Ile Lys Ala Pro Ala His Leu Val Trp Ser Leu Val Arg
                35 40 45
Arg Phe Asp Gln Pro Gln Arg Tyr Lys Pro Phe Val Ser Arg Cys Val
        50 55 60
Met Asn Gly Glu Leu Gly Ile Gly Ser Val Arg Glu Val Asn Val Lys
65 70 75 80
Ser Gly Leu Pro Ala Thr Thr Ser Thr Glu Arg Leu Glu Leu Leu Asp
                                85 90 95
Asp Glu Glu His Ile Leu Gly Val Gln Ile Val Gly Gly Asp His Arg
                        100 105 110
Leu Lys Asn Tyr Ser Ser Ile Met Thr Val His Pro Glu Phe Ile Asp
                115 120 125
Gly Arg Pro Gly Thr Leu Val Ile Glu Ser Phe Ile Val Asp Val Pro
        130 135 140
Asp Gly Asn Thr Lys Asp Glu Thr Cys Tyr Phe Val Lys Ala Leu Ile
145 150 155 160
Arg Cys Asn Leu Lys Ser Leu Ala Asp Val Ser Glu Arg Met Ala Val
                                165 170 175
Gln Asp Arg Val Glu Pro Val Asn Gln Phe
                        180 185
```

| | |
|---|---|
| <210> | 57 |
| <211> | 188 |
| <212> | PRT |
| <213> | Pisum sativum |
| <220> | |
| <223> | pea AT-rich element binding factor 3 (PsATF, ATF3), |
| | potential transcription factor for PsCHS1, GenBank |
| | Accession No. AAV85853.1, GI:56384584 |
| <400> | 57 |

```
Met Asn Asn Gly Gly Glu Gln Tyr Ser Ala Ile Glu Thr Gln Tyr Ile
  1 5 10 15
Arg Arg Arg His Lys His Asp Leu Arg Asp Asn Gln Cys Ser Ser Ala
                        20 25 30
Leu Val Lys His Ile Lys Ala Pro Val His Leu Val Trp Ser Leu Val
                35 40 45
Arg Arg Phe Asp Gln Pro Gln Lys Tyr Lys Pro Phe Val Ser Arg Cys
        50 55 60
Ile Met Gln Gly Asp Leu Gly Ile Gly Ser Val Arg Glu Val Asn Val
65 70 75 80
Lys Ser Gly Leu Pro Ala Thr Thr Ser Thr Glu Arg Leu Glu Gln Leu
                                85 90 95
Asp Asp Glu Glu His Ile Leu Gly Ile Arg Ile Val Gly Gly Asp His
                        100 105 110
Arg Leu Arg Asn Tyr Ser Ser Val Ile Thr Val His Pro Glu Val Ile
                115 120 125
Asp Gly Arg Pro Gly Thr Met Val Ile Glu Ser Phe Val Val Asp Val
        130 135 140
Pro Glu Gly Asn Thr Arg Asp Glu Thr Cys Tyr Phe Val Glu Ala Leu
145 150 155 160
Ile Arg Gly Asn Leu Ser Ser Leu Ala Asp Val Ser Glu Arg Met Ala
                                165 170 175
Val Gln Gly Arg Thr Asp Pro Ile Asn Val Asn Pro
                        180 185
```

| | |
|---|---|
| <210> | 58 |
| <211> | 177 |
| <212> | PRT |
| <213> | Vitis vinifera |
| <220> | |
| <223> | wine grape cuitivar PN40024 unnamed protein product, locus |
| | tag GSVIVT00027009001, GenBank Accession No. CAO39744.1, |
| | GI:157349888 |
| <400> | 58 |

```
Met Glu Ala Gln Val Ile Cys Arg His His Ala His Glu Pro Arg Glu
  1 5 10 15
Asn Gln Cys Ser Ser Val Leu Val Arg His Val Lys Ala Pro Ala Asn
                        20 25 30
Leu Val Trp Ser Leu Val Arg Arg Phe Asp Gln Pro Gln Lys Tyr Lys
                35 40 45
Pro Phe Val Ser Arg Cys Val Val Gln Gly Asp Leu Arg Ile Gly Ser
        50 55 60
Val Arg Glu Val Asn Val Lys Thr Gly Leu Pro Ala Thr Thr Ser Thr
65 70 75 80
Glu Arg Leu Glu Leu Phe Asp Asp Asp Glu His Val Leu Gly Ile Lys
                                85 90 95
Ile Leu Asp Gly Asp His Arg Leu Arg Asn Tyr Ser Ser Val Ile Thr
                        100 105 110
Val His Pro Glu Ile Ile Asp Gly Arg Pro Gly Thr Leu Val Ile Glu
                115 120 125
Ser Phe Val Val Asp Val Pro Glu Gly Asn Thr Lys Asp Asp Thr Cys
        130 135 140
Tyr Phe Val Arg Ala Leu Ile Asn Cys Asn Leu Lys Cys Leu Ala Glu
145 150 155 160
Val Ser Glu Arg Met Ala Met Leu Gly Arg Val Glu Pro Ala Asn Ala
                                165 170 175
Val
```

| | |
|---|---|
| <210> | 59 |
| <211> | 178 |
| <212> | PRT |
| <213> | Vitis vinifera |
| <220> | |
| <223> | wine grape cultivar Pinot Noir hypothetical protein, clone |
| | ENTAV 115, locus tag VITISV_004915, GenBank Accession No. |
| | CAN82501.1, GI:147856414 |
| <400> | 59 |

```
Met Met Glu Ala Gln Val Ile Cys Arg His His Ala His Glu Pro Arg
  1 5 10 15
Glu Asn Gln Cys Ser Ser Val Leu Val Arg His Val Lys Ala Pro Ala
                        20 25 30
Asn Leu Val Trp Ser Leu Val Arg Arg Phe Asp Gln Pro Gln Lys Tyr
                35 40 45
Lys Pro Phe Val Ser Arg Cys Val Val Gln Gly Asp Leu Arg Ile Gly
        50 55 60
Ser Val Arg Glu Val Asn Val Lys Thr Gly Leu Pro Ala Thr Thr Ser
65 70 75 80
Thr Glu Arg Leu Glu Leu Phe Asp Asp Asp Glu His Val Leu Gly Ile
                                85 90 95
Lys Ile Leu Asp Gly Asp His Arg Leu Arg Asn Tyr Ser Ser Val Ile
                        100 105 110
Thr Val His Pro Glu Ile Ile Asp Gly Arg Pro Gly Thr Leu Val Ile
                115 120 125
Glu Ser Phe Val Val Asp Val Pro Glu Gly Asn Thr Lys Asp Asp Thr
        130 135 140
Cys Tyr Phe Val Arg Ala Leu Ile Asn Cys Asn Leu Lys Cys Leu Ala
145 150 155 160
Glu Val Ser Glu Arg Met Ala Met Leu Gly Arg Val Glu Pro Ala Asn
                                165 170 175
Ala Val
```

| | |
|---|---|
| <210> | 60 |
| <211> | 193 |
| <212> | PRT |
| <213> | Arachis hypogaea |
| <220> | |
| <223> | peanut pathogenesis-induced protein (PIP), GenBank |
| | Accession No. ACG76109.1, GI:196196276 |
| <220> | |
| <221> | VARIANT |
| <222> | (162)...(162) |
| <223> | Xaa = any amino acid |
| <400> | 60 |

```
Met Met Asn Gly Ser Cys Gly Gly Gly Gly Gly Gly Glu Ala Tyr Gly
  1 5 10 15
Ala Ile Glu Ala Gln Tyr Ile Arg Arg His His Arg His Glu Pro Arg
                        20 25 30
Asp Asn Gln Cys Thr Ser Ala Leu Val Lys His Ile Arg Ala Pro Val
                35 40 45
His Leu Val Trp Ser Leu Val Arg Arg Phe Asp Gln Pro Gln Lys Tyr
        50 55 60
Lys Pro Phe Val Ser Arg Cys Ile Met Gln Gly Asp Leu Gly Ile Gly
65 70 75 80
Ser Val Arg Glu Val Asn Val Lys Ser Gly Leu Pro Ala Thr Thr Ser
                                85 90 95
Thr Glu Arg Leu Glu Gln Leu Asp Asp Glu Glu His Ile Leu Gly Ile
                        100 105 110
Arg Ile Val Gly Gly Asp His Arg Leu Arg Asn Tyr Ser Ser Ile Ile
                115 120 125
Thr Val His Pro Glu Val Ile Glu Gly Arg Pro Gly Thr Met Val Ile
        130 135 140
Glu Ser Phe Val Val Asp Val Pro Asp Gly Asn Thr Lys Asp Glu Thr
145 150 155 160
Cys Xaa Phe Val Glu Ala Leu Ile Arg Cys Asn Leu Ser Ser Leu Ala
                                165 170 175
Asp Val Ser Glu Arg Met Ala Val Gln Gly Arg Thr Asp Pro Ile Asn
                        180 185 190
Gln
```

| | |
|---|---|
| <210> | 61 |
| <211> | 217 |
| <212> | PRT |
| <213> | Zea mays |
| <220> | |
| <223> | maize AT-rich element binding factor 3, clone 300908, |
| | GenBank Accession No. ACG39386.1, GI:195639836 |
| <400> | 61 |

```
Met Val Val Glu Met Asp Gly Gly Val Gly Val Ala Ala Gly Gly Gly
  1 5 10 15
Gly Gly Ala Gln Thr Pro Ala Pro Ala Pro Pro Arg Arg Trp Arg Leu
                        20 25 30
Ala Asp Glu Arg Cys Asp Leu Arg Ala Met Glu Thr Asp Tyr Val Arg
                35 40 45
Arg Phe His Arg His Glu Pro Arg Asp His Gln Cys Ser Ser Ala Val
        50 55 60
Ala Lys His Ile Lys Ala Pro Val His Leu Val Trp Ser Leu Val Arg
65 70 75 80
Arg Phe Asp Gln Pro Gln Leu Phe Lys Pro Phe Val Ser Arg Cys Glu
                                85 90 95
Met Lys Gly Asn Ile Glu Ile Gly Ser Val Arg Glu Val Asn Val Lys
                        100 105 110
Ser Gly Leu Pro Ala Thr Arg Ser Thr Glu Arg Leu Glu Leu Leu Asp
                115 120 125
Asp Asp Glu Arg Ile Leu Ser Val Arg Phe Val Gly Gly Asp His Arg
        130 135 140
Leu Gln Asn Tyr Ser Ser Ile Leu Thr Val His Pro Glu Val Ile Asp
145 150 155 160
Gly Arg Pro Gly Thr Leu Val Ile Glu Ser Phe Val Val Asp Val Pro
                                165 170 175
Asp Gly Asn Thr Lys Asp Glu Thr Cys Tyr Phe Val Glu Ala Leu Leu
                        180 185 190
Lys Cys Asn Leu Arg Ser Leu Ala Glu Val Ser Glu Gly Gln Val Ile
                195 200 205
Met Asp Gln Thr Glu Pro Leu Asp Arg
        210 215
```

| | |
|---|---|
| <210> | 62 |
| <211> | 217 |
| <212> | PRT |
| <213> | Zea mays |
| <220> | |
| <223> | maize strain B73, unknown protein, clone ZM_BFb0036A01, |
| | GenBank Accession No. ACF80077.1, GI:194691986 |
| <400> | 62 |

```
Met Val Val Glu Met Asp Gly Gly Val Gly Val Ala Ala Ala Gly Gly
  1 5 10 15
Gly Gly Ala Gln Thr Pro Ala Pro Pro Pro Pro Arg Arg Trp Arg Leu
                        20 25 30
Ala Asp Glu Arg Cys Asp Leu Arg Ala Met Glu Thr Asp Tyr Val Arg
                35 40 45
Arg Phe His Arg His Glu Pro Arg Asp His Gln Cys Ser Ser Ala Val
        50 55 60
Ala Lys His Ile Lys Ala Pro Val His Leu Val Trp Ser Leu Val Arg
65 70 75 80
Arg Phe Asp Gln Pro Gln Leu Phe Lys Pro Phe Val Ser Arg Cys Glu
                                85 90 95
Met Lys Gly Asn Ile Glu Ile Gly Ser Val Arg Glu Val Asn Val Lys
                        100 105 110
Ser Gly Leu Pro Ala Thr Arg Ser Thr Glu Arg Leu Glu Leu Leu Asp
                115 120 125
Asp Asp Glu Arg Ile Leu Ser Val Arg Phe Val Gly Gly Asp His Arg
        130 135 140
Leu Gln Asn Tyr Ser Ser Ile Leu Thr Val His Pro Glu Val Ile Asp
145 150 155 160
Gly Arg Pro Gly Thr Leu Val Ile Glu Ser Phe Val Val Asp Val Pro
                                165 170 175
Asp Gly Asn Thr Lys Asp Glu Thr Cys Tyr Phe Val Glu Ala Leu Leu
                        180 185 190
Lys Cys Asn Leu Arg Ser Leu Ala Glu Val Ser Glu Gly Gln Val Ile
                195 200 205
Met Asp Gln Thr Glu Pro Leu Asp Arg
        210 215
```

| | |
|---|---|
| <210> | 63 |
| <211> | 206 |
| <212> | PRT |
| <213> | Oryza sativa |
| <220> | |
| <223> | rice Japonica Group, cultivar Nipponbare, conserved |
| | hypothetical protein Os06g0528300, GenBank Accession No. |
| | NP_001057772.1, GI:115468346 |
| <400> | 63 |

```
Met Asn Gly Val Gly Gly Ala Gly Gly Ala Ala Ala Gly Lys Leu Pro
  1 5 10 15
Met Val Ser His Arg Arg Val Gln Trp Arg Leu Ala Asp Glu Arg Cys
                        20 25 30
Glu Leu Arg Glu Glu Glu Met Glu Tyr Ile Arg Arg Phe His Arg His
                35 40 45
Glu Pro Ser Ser Asn Gln Cys Thr Ser Phe Ala Ala Lys His Ile Lys
        50 55 60
Ala Pro Leu His Thr Val Trp Ser Leu Val Arg Arg Phe Asp Gln Pro
65 70 75 80
Gln Leu Phe Lys Pro Phe Val Arg Asn Cys Val Met Arg Glu Asn Ile
                                85 90 95
Ile Ala Thr Gly Cys Ile Arg Glu Val Asn Val Gln Ser Gly Leu Pro
                        100 105 110
Ala Thr Arg Ser Thr Glu Arg Leu Glu Leu Leu Asp Asp Asn Glu His
                115 120 125
Ile Leu Lys Val Asn Phe Ile Gly Gly Asp His Met Leu Lys Asn Tyr
        130 135 140
Ser Ser Ile Leu Thr Val His Ser Glu Val Ile Asp Gly Gln Leu Gly
145 150 155 160
Thr Leu Val Val Glu Ser Phe Ile Val Asp Val Pro Glu Gly Asn Thr
                                165 170 175
Lys Asp Asp Ile Ser Tyr Phe Ile Glu Asn Val Leu Arg Cys Asn Leu
                        180 185 190
Arg Thr Leu Ala Asp Val Ser Glu Glu Arg Leu Ala Asn Pro
                195 200 205
```

| | |
|---|---|
| <210> | 64 |
| <211> | 206 |
| <212> | PRT |
| <213> | Oryza sativa |
| <220> | |
| <223> | rice Indica Group, cultivar 93-11, hypothetical protein |
| | OsI_23215, old locus tag OsI_022420, GLEAN gene, GenBank |
| | Accession No. EAZ01188.1, GI:125555582 |
| <400> | 64 |

```
Met Asn Gly Ala Gly Gly Ala Gly Gly Ala Ala Ala Gly Lys Leu Pro
  1 5 10 15
Met Val Ser His Arg Gln Val Gln Trp Arg Leu Ala Asp Glu Arg Cys
                        20 25 30
Glu Leu Arg Glu Glu Glu Met Glu Tyr Ile Arg Gln Phe His Arg His
                35 40 45
Glu Pro Ser Ser Asn Gln Cys Thr Ser Phe Val Ala Lys His Ile Lys
        50 55 60
Ala Pro Leu Gln Thr Val Trp Ser Leu Val Arg Arg Phe Asp Gln Pro
65 70 75 80
Gln Leu Phe Lys Pro Phe Val Arg Lys Cys Val Met Arg Glu Asn Ile
                                85 90 95
Ile Ala Thr Gly Cys Val Arg Glu Val Asn Val Gln Ser Gly Leu Pro
                        100 105 110
Ala Thr Arg Ser Thr Glu Arg Leu Glu Leu Leu Asp Asp Asn Glu His
                115 120 125
Ile Leu Lys Val Lys Phe Ile Gly Gly Asp His Met Leu Lys Asn Tyr
        130 135 140
Ser Ser Ile Leu Thr Ile His Ser Glu Val Ile Asp Gly Gln Leu Gly
145 150 155 160
Thr Leu Val Val Glu Ser Phe Val Val Asp Ile Pro Glu Gly Asn Thr
                                165 170 175
Lys Asp Asp Ile Cys Tyr Phe Ile Glu Asn Ile Leu Arg Cys Asn Leu
                        180 185 190
Met Thr Leu Ala Asp Val Ser Glu Glu Arg Leu Ala Asn Pro
                195 200 205
```

| | |
|---|---|
| <210> | 65 |
| <211> | 205 |
| <212> | PRT |
| <213> | Oryza sativa |
| <220> | |
| <223> | rice Japonica Group, cultivar Nipponbare, hypothetical |
| | protein OsJ_06125, old locus tag OsI_005939, GenBank |
| | Accession No. EAZ22456.1, GI:125581525 |
| <400> | 65 |

```
Met Val Glu Val Gly Gly Gly Ala Ala Glu Ala Ala Ala Gly Arg Arg
  1 5 10 15
Trp Arg Leu Ala Asp Glu Arg Cys Asp Leu Arg Ala Ala Glu Thr Glu
                        20 25 30
Tyr Val Arg Arg Phe His Arg His Glu Pro Arg Asp His Gln Cys Ser
                35 40 45
Ser Ala Val Ala Lys His Ile Lys Ala Pro Val His Leu Val Trp Ser
        50 55 60
Leu Val Arg Arg Phe Asp Gln Pro Gln Leu Phe Lys Pro Phe Val Ser
65 70 75 80
Arg Cys Glu Met Lys Gly Asn Ile Glu Ile Gly Ser Val Arg Glu Val
                                85 90 95
Asn Val Lys Ser Gly Leu Pro Ala Thr Arg Ser Thr Glu Arg Leu Glu
                        100 105 110
Leu Leu Asp Asp Asn Glu His Ile Leu Ser Val Arg Phe Val Gly Gly
                115 120 125
Asp His Arg Leu Lys Asn Tyr Ser Ser Ile Leu Thr Val His Pro Glu
        130 135 140
Val Ile Asp Gly Arg Pro Gly Thr Leu Val Ile Glu Ser Phe Val Val
145 150 155 160
Asp Val Pro Glu Gly Asn Thr Lys Asp Glu Thr Cys Tyr Phe Val Glu
                                165 170 175
Ala Leu Leu Lys Cys Asn Leu Lys Ser Leu Ala Glu Val Ser Glu Arg
                        180 185 190
Leu Val Cys Gln Gly Pro Asn Arg Ala Pro Ser Thr Arg
                195 200 205
```

| | |
|---|---|
| <210> | 66 |
| <211> | 204 |
| <212> | PRT |
| <213> | Oryza sativa |
| <220> | |
| <223> | rice Japonica Group, cultivar Nipponbare, hypothetical |
| | protein Os02g0255500, similar to extensin (fragment), |
| | GenBank Accession No. NP_001046464.1, GI:115445369 |
| <400> | 66 |

```
Met Val Glu Val Gly Gly Gly Ala Ala Glu Ala Ala Ala Gly Arg Arg
  1 5 10 15
Trp Arg Leu Ala Asp Glu Arg Cys Asp Leu Arg Ala Ala Glu Thr Glu
                        20 25 30
Tyr Val Arg Arg Phe His Arg His Glu Pro Arg Asp His Gln Cys Ser
                35 40 45
Ser Ala Val Ala Lys His Ile Lys Ala Pro Val His Leu Val Trp Ser
        50 55 60
Leu Val Arg Arg Phe Asp Gln Pro Gln Leu Phe Lys Pro Phe Val Ser
65 70 75 80
Arg Cys Glu Met Lys Gly Asn Ile Glu Ile Gly Ser Val Arg Glu Val
                                85 90 95
Asn Val Lys Ser Gly Leu Pro Ala Thr Arg Ser Thr Glu Arg Leu Glu
                        100 105 110
Leu Leu Asp Asp Asn Glu His Ile Leu Ser Val Arg Phe Val Gly Gly
                115 120 125
Asp His Arg Leu Lys Asn Tyr Ser Ser Ile Leu Thr Val His Pro Glu
        130 135 140
Val Ile Asp Gly Arg Pro Gly Thr Leu Val Ile Glu Ser Phe Val Val
145 150 155 160
Asp Val Pro Glu Gly Asn Thr Lys Asp Glu Thr Cys Tyr Phe Val Glu
                                165 170 175
Ala Leu Leu Lys Cys Asn Leu Lys Ser Leu Ala Glu Val Ser Glu Arg
                        180 185 190
Leu Val Val Lys Asp Gln Thr Glu Pro Leu Asp Arg
                195 200
```

| | |
|---|---|
| <210> | 67 |
| <211> | 199 |
| <212> | PRT |
| <213> | Medicago truncatula |
| <220> | |
| <223> | barrel medic unknown protein, clone MTYFP_FQ_FR_FS1G-G-11, |
| | GenBank Accession No. ACJ86004.1, GI:217075288 |
| <400> | 67 |

```
Met Glu Lys Met Asn Gly Thr Glu Asn Asn Gly Val Phe Asn Ser Thr
  1 5 10 15
Glu Met Glu Tyr Ile Arg Arg His His Asn Gln Gln Pro Gly Glu Asn
                        20 25 30
Gln Cys Ser Ser Ala Leu Val Lys His Ile Arg Ala Pro Val Pro Leu
                35 40 45
Val Trp Ser Leu Val Arg Arg Phe Asp Gln Pro Gln Lys Tyr Lys Pro
        50 55 60
Phe Val Ser Arg Cys Val Val Arg Gly Asn Leu Glu Ile Gly Ser Leu
65 70 75 80
Arg Glu Val Asp Val Lys Ser Gly Leu Pro Ala Thr Thr Ser Thr Glu
                                85 90 95
Arg Leu Glu Val Leu Asp Asp Asn Glu His Ile Leu Ser Ile Arg Ile
                        100 105 110
Ile Gly Gly Asp His Arg Leu Arg Asn Tyr Ser Ser Ile Met Ser Leu
                115 120 125
His Pro Glu Ile Ile Asp Gly Arg Pro Gly Thr Leu Val Ile Glu Ser
        130 135 140
Phe Val Val Asp Val Pro Glu Gly Asn Thr Lys Asp Glu Thr Cys Tyr
145 150 155 160
Phe Val Glu Ala Leu Ile Lys Cys Asn Leu Lys Ser Leu Ser Asp Val
                                165 170 175
Ser Glu Gly His Ala Val Gln Asp Leu Thr Glu Pro Leu Asp Arg Val
                        180 185 190
His Glu Leu Leu Ile Ser Gly
                195
```

| | |
|---|---|
| <210> | 68 |
| <211> | 199 |
| <212> | PRT |
| <213> | Medicago truncatula |
| <220> | |
| <223> | barrel medic unknown protein, clone MTYF1_F2_F3_FY1G-K-4, |
| | GenBank Accession No. ACJ83958.1, GI:217071196 |
| <400> | 68 |

```
Met Glu Lys Met Asn Gly Thr Glu Asn Asn Gly Val Phe Asn Ser Thr
  1 5 10 15
Glu Met Glu Tyr Ile Arg Arg His His Asn Gln Gln Pro Gly Glu Asn
                        20 25 30
Gln Cys Ser Ser Ala Leu Val Lys His Ile Arg Ala Pro Val Pro Leu
                35 40 45
Val Trp Ser Leu Val Arg Arg Phe Asp Gln Pro Gln Lys Tyr Lys Pro
        50 55 60
Phe Val Ser Arg Cys Val Val Arg Gly Asn Leu Glu Ile Gly Ser Leu
65 70 75 80
Arg Glu Val Asp Val Lys Ser Gly Leu Pro Ala Thr Thr Ser Thr Glu
                                85 90 95
Arg Leu Glu Val Leu Asp Asp Asn Glu His Ile Leu Ser Ile Arg Ile
                        100 105 110
Ile Gly Gly Asp His Arg Leu Arg Asn Tyr Ser Ser Ile Met Ser Leu
                115 120 125
His Pro Glu Ile Ile Asp Gly Arg Pro Gly Thr Leu Val Ile Glu Ser
        130 135 140
Phe Val Val Asp Val Pro Glu Gly Asn Thr Lys Asp Glu Thr Cys Tyr
145 150 155 160
Phe Val Glu Ala Leu Ile Lys Cys Asn Leu Lys Ser Leu Ser Asp Val
                                165 170 175
Ser Glu Gly His Ala Ala Gln Asp Leu Thr Glu Pro Leu Asp Arg Met
                        180 185 190
His Glu Leu Leu Ile Ser Gly
                195
```

| | |
|---|---|
| <210> | 69 |
| <211> | 197 |
| <212> | PRT |
| <213> | Zea mays |
| <220> | |
| <223> | maize CAPIP1 protein, clone 244179, GenBank Accession No. |
| | ACG34726.1, GI:195625792 |
| <400> | 69 |

```
Met Val Gly Leu Val Gly Gly Ser Thr Ala Arg Ala Glu His Val Val
  1 5 10 15
Ala Asn Ala Gly Gly Glu Ala Glu Tyr Val Arg Arg Met His Arg His
                        20 25 30
Ala Pro Thr Glu His Gln Cys Thr Ser Thr Leu Val Lys His Ile Lys
                35 40 45
Ala Pro Val His Leu Val Trp Gln Leu Val Arg Arg Phe Asp Gln Pro
        50 55 60
Gln Arg Tyr Lys Pro Phe Val Arg Asn Cys Val Val Arg Gly Asp Gln
65 70 75 80
Leu Glu Val Gly Ser Leu Arg Asp Val Asn Val Lys Thr Gly Leu Pro
                                85 90 95
Ala Thr Thr Ser Thr Glu Arg Leu Glu Gln Leu Asp Asp Asp Leu His
                        100 105 110
Ile Leu Gly Val Lys Phe Val Gly Gly Asp His Arg Leu Gln Asn Tyr
                115 120 125
Ser Ser Ile Ile Thr Val His Pro Glu Ser Ile Asp Gly Arg Pro Gly
        130 135 140
Thr Leu Val Ile Glu Ser Phe Val Val Asp Val Pro Asp Gly Asn Thr
145 150 155 160
Lys Asp Glu Thr Cys Tyr Phe Val Glu Ala Val Ile Lys Cys Asn Leu
                                165 170 175
Asn Ser Leu Ala Glu Val Ser Glu Gln Leu Ala Val Glu Ser Pro Thr
                        180 185 190
Ser Leu Ile Asp Gln
                195
```

| | |
|---|---|
| <210> | 70 |
| <211> | 197 |
| <212> | PRT |
| <213> | Zea mays |
| <220> | |
| <223> | maize CAPIP1 protein, clone 1448906, GenBank Accession No. |
| | ACG26022.1, GI:195608384 |
| <400> | 70 |

```
Met Val Gly Leu Val Gly Gly Ser Thr Ala Arg Ala Glu His Val Val
  1 5 10 15
Ala Asn Ala Gly Gly Glu Ala Glu Tyr Val Arg Arg Met His Arg His
                        20 25 30
Ala Pro Thr Glu His Gln Cys Thr Ser Thr Leu Val Lys His Ile Lys
                35 40 45
Ala Pro Val His Leu Val Trp Glu Leu Val Arg Arg Phe Asp Gln Pro
        50 55 60
Gln Arg Tyr Lys Pro Phe Val Arg Asn Cys Val Val Arg Gly Asp Gln
65 70 75 80
Leu Glu Val Gly Ser Leu Arg Asp Val Asn Val Lys Thr Gly Leu Pro
                                85 90 95
Ala Thr Thr Ser Thr Glu Arg Leu Glu Gln Leu Asp Asp Asp Leu His
                        100 105 110
Ile Leu Gly Val Lys Phe Val Gly Gly Asp His Arg Leu Gln Asn Tyr
                115 120 125
Ser Ser Ile Ile Thr Val His Pro Glu Ser Ile Asp Gly Arg Pro Gly
        130 135 140
Thr Leu Val Ile Glu Ser Phe Val Val Asp Val Pro Asp Gly Asn Thr
145 150 155 160
Lys Asp Glu Thr Cys Tyr Phe Val Glu Ala Val Ile Lys Cys Asn Leu
                                165 170 175
Asn Ser Leu Ala Glu Val Ser Glu Gln Leu Ala Val Glu Ser Pro Thr
                        180 185 190
Ser Leu Ile Asp Gln
                195
```

| | |
|---|---|
| <210> | 71 |
| <211> | 212 |
| <212> | PRT |
| <213> | Zea mays |
| <220> | |
| <223> | maize strain B73 unknown protein, clone ZM_BFc0183D21, |
| | GenBank Accession No. ACF86162.1, GI:194704156 |
| <400> | 71 |

```
Met Val Met Val Glu Met Asp Gly Gly Val Gly Gly Gly Gly Gly Gly
  1 5 10 15
Gly Gln Thr Pro Ala Pro Arg Arg Trp Arg Leu Ala Asp Glu Arg Cys
                        20 25 30
Asp Leu Arg Ala Met Glu Thr Asp Tyr Val Arg Arg Phe His Arg His
                35 40 45
Glu Pro Arg Glu His Gln Cys Ser Ser Ala Val Ala Lys His Ile Lys
        50 55 60
Ala Pro Val His Leu Val Trp Ser Leu Val Arg Arg Phe Asp Gln Pro
65 70 75 80
Gln Leu Phe Lys Pro Phe Val Ser Arg Cys Glu Met Lys Gly Asn Ile
                                85 90 95
Glu Ile Gly Ser Val Arg Glu Val Asn Val Lys Ser Gly Leu Pro Ala
                        100 105 110
Thr Arg Ser Thr Glu Arg Leu Glu Leu Leu Asp Asp Asn Glu His Ile
                115 120 125
Leu Ser Val Arg Phe Val Gly Gly Asp His Arg Leu Gln Asn Tyr Ser
        130 135 140
Ser Ile Leu Thr Val His Pro Glu Val Ile Asp Gly Arg Pro Gly Thr
145 150 155 160
Leu Val Ile Glu Ser Phe Val Val Asp Val Pro Asp Gly Asn Thr Lys
                                165 170 175
Asp Glu Thr Cys Tyr Phe Val Glu Ala Leu Leu Lys Cys Asn Leu Lys
                        180 185 190
Ser Leu Ala Glu Val Ser Glu Arg Gln Val Val Lys Asp Gln Thr Glu
                195 200 205
Pro Leu Asp Arg
        210
```

| | |
|---|---|
| <210> | 72 |
| <211> | 205 |
| <212> | PRT |
| <213> | Oryza sativa |
| <220> | |
| <223> | rice Japonica Group, cultivar Nipponbare, conserved |
| | hypothetical protein Os06g0527800, GenBank Accession No. |
| | NP_001057771.1, GI:115468344 |
| <400> | 72 |

```
Met Asn Gly Ala Gly Gly Ala Gly Gly Ala Ala Ala Gly Lys Leu Pro
  1 5 10 15
Met Val Ser His Arg Arg Val Gln Cys Arg Leu Ala Asp Lys Arg Cys
                        20 25 30
Glu Leu Arg Glu Glu Glu Met Glu Tyr Ile Arg Gln Phe His Arg His
                35 40 45
Glu Pro Ser Ser Asn Gln Cys Thr Ser Phe Val Ala Lys His Ile Lys
        50 55 60
Ala Pro Leu Gln Thr Val Trp Ser Leu Val Arg Arg Phe Asp Gln Pro
65 70 75 80
Gln Leu Phe Lys Pro Phe Val Arg Lys Cys Val Met Arg Glu Asn Ile
                                85 90 95
Ile Val Thr Gly Cys Val Arg Glu Val Asn Val Gln Ser Gly Leu Pro
                        100 105 110
Ala Thr Arg Ser Thr Glu Arg Leu Glu Leu Leu Asp Asp Asn Glu His
                115 120 125
Ile Leu Lys Val Lys Phe Ile Gly Gly Asp His Met Leu Lys Asn Tyr
        130 135 140
Ser Ser Ile Leu Thr Ile His Ser Glu Val Ile Asp Gly Gln Leu Gly
145 150 155 160
Thr Leu Val Val Glu Ser Phe Val Val Asp Ile Pro Asp Gly Asn Thr
                                165 170 175
Lys Asp Asp Ile Cys Tyr Phe Ile Glu Asn Val Leu Arg Cys Asn Leu
                        180 185 190
Met Thr Leu Ala Asp Val Ser Glu Glu Arg Leu Ala Asn
                195 200 205
```

| | |
|---|---|
| <210> | 73 |
| <211> | 197 |
| <212> | PRT |
| <213> | Zea mays |
| <220> | |
| <223> | maize strain B73 unknown protein, clone ZM_BFc0063E17, |
| | GenBank Accession No. ACF85073.1, GI:194701978 |
| <400> | 73 |

```
Met Val Gly Leu Val Gly Gly Ser Thr Ala Arg Ala Glu His Val Val
  1 5 10 15
Ala Asn Ala Gly Gly Glu Thr Glu Tyr Val Arg Arg Leu His Arg His
                        20 25 30
Ala Pro Ala Glu His Gln Cys Thr Ser Thr Leu Val Lys His Ile Lys
                35 40 45
Ala Pro Val His Leu Val Trp Glu Leu Val Arg Ser Phe Asp Gln Pro
        50 55 60
Gln Arg Tyr Lys Pro Phe Val Arg Asn Cys Val Val Arg Gly Asp Gln
65 70 75 80
Leu Glu Val Gly Ser Leu Arg Asp Val Asn Val Lys Thr Gly Leu Pro
                                85 90 95
Ala Thr Thr Ser Thr Glu Arg Leu Glu Gln Leu Asp Asp Asp Leu His
                        100 105 110
Ile Leu Gly Val Lys Phe Val Gly Gly Asp His Arg Leu Gln Asn Tyr
                115 120 125
Ser Ser Ile Ile Thr Val His Pro Glu Ser Ile Asp Gly Arg Pro Gly
        130 135 140
Thr Leu Val Ile Glu Ser Phe Val Val Asp Val Pro Asp Gly Asn Thr
145 150 155 160
Lys Asp Glu Thr Cys Tyr Phe Val Glu Ala Val Ile Lys Cys Asn Leu
                                165 170 175
Lys Ser Leu Ala Glu Val Ser Glu Gln Leu Ala Val Glu Ser Pro Thr
                        180 185 190
Ser Pro Ile Asp Gln
                195
```

| | |
|---|---|
| <210> | 74 |
| <211> | 206 |
| <212> | PRT |
| <213> | Oryza sativa |
| <220> | |
| <223> | rice Indica Group, cultivar 93-11, hypothetical protein |
| | OsI_23218, old locus tag OsI_022423, GLEAN gene, GenBank |
| | Accession No. EAZ01191.1, GI:125555585 |
| <400> | 74 |

```
Met Asn Gly Val Gly Gly Ala Gly Gly Ala Ala Ala Gly Lys Leu Pro
  1 5 10 15
Met Val Ser His Arg Arg Val Gln Trp Arg Leu Ala Asp Glu Arg Cys
                        20 25 30
Glu Leu Arg Glu Glu Glu Met Glu Tyr Ile Arg Arg Phe His Arg His
                35 40 45
Glu Pro Ser Ser Asn Gln Cys Thr Ser Phe Ala Ala Lys His Ile Lys
        50 55 60
Ala Pro Leu His Thr Val Trp Ser Leu Val Arg Arg Phe Asp Gln Pro
65 70 75 80
Gln Leu Phe Lys Pro Phe Val Arg Asn Cys Val Met Arg Glu Asn Ile
                                85 90 95
Ile Ala Thr Gly Cys Ile Arg Glu Val Asn Val Gln Ser Gly Leu Pro
                        100 105 110
Ala Thr Arg Ser Thr Glu Arg Leu Glu Leu Leu Asp Asp Asn Glu His
                115 120 125
Ile Leu Lys Val Lys Phe Ile Gly Gly Asp His Met Leu Lys Asn Tyr
        130 135 140
Ser Ser Ile Leu Thr Val His Ser Glu Val Ile Asp Gly Gln Leu Gly
145 150 155 160
Thr Leu Val Val Glu Ser Phe Ile Val Asp Val Leu Glu Gly Asn Thr
                                165 170 175
Lys Asp Asp Ile Ser Tyr Phe Ile Glu Asn Val Leu Arg Cys Asn Leu
                        180 185 190
Arg Thr Leu Ala Asp Val Ser Glu Glu Arg Leu Ala Asn Pro
                195 200 205
```

| | |
|---|---|
| <210> | 75 |
| <211> | 209 |
| <212> | PRT |
| <213> | Oryza sativa |
| <220> | |
| <223> | rice Japonica Group, cultivar Nipponbare, conserved |
| | hypothetical protein Os05g0213500, GenBank Accession No. |
| | NP_001054923.1, GI:115462647 |
| <400> | 75 |

```
Met Val Gly Leu Val Gly Gly Gly Gly Trp Arg Val Gly Asp Asp Ala
  1 5 10 15
Ala Gly Gly Gly Gly Gly Gly Ala Val Ala Ala Gly Ala Ala Ala Ala
                        20 25 30
Ala Glu Ala Glu His Met Arg Arg Leu His Ser His Ala Pro Gly Glu
                35 40 45
His Gln Cys Ser Ser Ala Leu Val Lys His Ile Lys Ala Pro Val His
        50 55 60
Leu Val Trp Ser Leu Val Arg Ser Phe Asp Gln Pro Gln Arg Tyr Lys
65 70 75 80
Pro Phe Val Ser Arg Cys Val Val Arg Gly Gly Asp Leu Glu Ile Gly
                                85 90 95
Ser Val Arg Glu Val Asn Val Lys Thr Gly Leu Pro Ala Thr Thr Ser
                        100 105 110
Thr Glu Arg Leu Glu Leu Leu Asp Asp Asp Glu His Ile Leu Ser Val
                115 120 125
Lys Phe Val Gly Gly Asp His Arg Leu Arg Asn Tyr Ser Ser Ile Val
        130 135 140
Thr Val His Pro Glu Ser Ile Asp Gly Arg Pro Gly Thr Leu Val Ile
145 150 155 160
Glu Ser Phe Val Val Asp Val Pro Asp Gly Asn Thr Lys Asp Glu Thr
                                165 170 175
Cys Tyr Phe Val Glu Ala Val Ile Lys Cys Asn Leu Thr Ser Leu Ala
                        180 185 190
Glu Val Ser Glu Arg Leu Ala Val Gln Ser Pro Thr Ser Pro Leu Glu
                195 200 205
Gln
```

| | |
|---|---|
| <210> | 76 |
| <211> | 180 |
| <212> | PRT |
| <213> | Oryza sativa |
| <220> | |
| <223> | rice Japonica Group, cultivar Nipponbare, Bet v I |
| | allergen-like protein, clone OSJNBa0052K15, gene |
| | OSJNBa0052K15.17, GenBank Accession No. BAD29692.1, |
| | GI:50251668 |
| <400> | 76 |

```
Met Val Glu Met Asp Ala Gly Gly Arg Pro Glu Pro Ser Pro Pro Ser
  1 5 10 15
Gly Gln Cys Ser Ser Ala Val Thr Met Arg Ile Asn Ala Pro Val His
                        20 25 30
Leu Val Trp Ser Ile Val Arg Arg Phe Glu Glu Pro His Ile Phe Gln
                35 40 45
Pro Phe Val Arg Gly Cys Thr Met Arg Gly Ser Thr Ser Leu Ala Val
        50 55 60
Gly Cys Val Arg Glu Val Asp Phe Lys Ser Gly Phe Pro Ala Lys Ser
65 70 75 80
Ser Val Glu Arg Leu Glu Ile Leu Asp Asp Lys Glu His Val Phe Gly
                                85 90 95
Val Arg Ile Ile Gly Gly Asp His Arg Leu Lys Asn Tyr Ser Ser Val
                        100 105 110
Leu Thr Ala Lys Pro Glu Val Ile Asp Gly Glu Pro Ala Thr Leu Val
                115 120 125
Ser Glu Ser Phe Val Val Asp Val Pro Glu Gly Asn Thr Ala Asp Glu
        130 135 140
Thr Arg His Phe Val Glu Phe Leu Ile Arg Cys Asn Leu Arg Ser Leu
145 150 155 160
Ala Met Val Ser Gln Arg Leu Leu Leu Ala Gln Gly Asp Leu Ala Glu
                                165 170 175
Pro Pro Ala Gln
                        180
```

| | |
|---|---|
| <210> | 77 |
| <211> | 176 |
| <212> | PRT |
| <213> | Vitis vinifera |
| <220> | |
| <223> | wine grape cultivar Pinot Noir hypothetical protein, clone |
| | ENTAV 115, locus tag VITISV_029498, GenBank Accession No. |
| | CAN64668.1, GI:147797548 |
| <400> | 77 |

```
Met Asn Gly Asn Gly Leu Ser Ser Met Glu Ser Glu Tyr Ile Arg Arg
  1 5 10 15
His His Arg His Glu Pro Ala Glu Asn Gln Cys Ser Ser Ala Leu Val
                        20 25 30
Lys His Ile Lys Ala Pro Val Pro Leu Val Trp Ser Leu Val Arg Arg
                35 40 45
Phe Asp Gln Pro Gln Lys Tyr Lys Pro Phe Ile Ser Arg Cys Val Val
        50 55 60
Gln Gly Asn Leu Glu Ile Gly Ser Leu Arg Glu Val Asp Val Lys Ser
65 70 75 80
Gly Leu Pro Ala Thr Thr Ser Thr Glu Arg Leu Glu Leu Leu Asp Asp
                                85 90 95
Asp Glu His Ile Leu Ser Met Arg Ile Ile Gly Gly Asp His Arg Leu
                        100 105 110
Arg Asn Tyr Ser Ser Ile Ile Ser Leu His Pro Glu Ile Ile Asp Gly
                115 120 125
Arg Pro Gly Thr Met Val Ile Glu Ser Tyr Val Val Asp Val Pro Glu
        130 135 140
Gly Asn Thr Lys Asp Glu Thr Cys Tyr Phe Ser Leu Ala Asp Val Ser
145 150 155 160
Glu Arg Leu Ala Val Ala Gly Thr Val Thr Glu Pro Ile Asp Arg Met
                                165 170 175
```

| | |
|---|---|
| <210> | 78 |
| <211> | 180 |
| <212> | PRT |
| <213> | Oryza sativa |
| <220> | |
| <223> | rice Indica Group, cultivar 93-11, hypothetical protein, |
| | locus tag OsI_06615, GLEAN gene, GenBank Accession No. |
| | EEC72859.1, GI:218190432 |
| <400> | 78 |

```
Met Val Glu Met Asp Ala Gly Gly Arg Pro Glu Pro Ser Pro Pro Ser
  1 5 10 15
Gly Gln Cys Ser Ser Ala Val Thr Met Arg Ile Asn Ala Pro Val His
                        20 25 30
Leu Val Trp Ser Ile Val Arg Arg Phe Glu Glu Pro His Ile Phe Gln
                35 40 45
Pro Phe Val Arg Gly Cys Thr Met Arg Gly Ser Thr Ser Leu Ala Val
        50 55 60
Gly Cys Val Arg Glu Val Asp Phe Lys Ser Gly Phe Ser Ala Lys Ser
65 70 75 80
Ser Val Glu Arg Leu Glu Ile Leu Asp Asp Lys Glu His Val Phe Gly
                                85 90 95
Val Arg Ile Ile Gly Gly Asp His Arg Leu Lys Asn Tyr Ser Ser Val
                        100 105 110
Leu Thr Ala Lys Pro Glu Val Ile Asp Gly Glu Pro Ala Thr Leu Val
                115 120 125
Ser Glu Ser Phe Val Ile Asp Val Pro Glu Gly Asn Thr Ala Asp Glu
        130 135 140
Thr Arg His Phe Val Glu Phe Leu Ile Arg Cys Asn Leu Arg Ser Leu
145 150 155 160
Ala Met Val Ser Gln Arg Leu Leu Leu Ala Gln Gly Asp Leu Ala Glu
                                165 170 175
Pro Pro Ala Gln
                        180
```

| | |
|---|---|
| <210> | 79 |
| <211> | 215 |
| <212> | PRT |
| <213> | Oryza sativa |
| <220> | |
| <223> | rice Japonica Group, cultivar Nipponbare, hypothetical |
| | protein OsJ_10498, old locus tag OsJ_010081, GLEAN gene, |
| | GenBank Accession No. EAZ26598.1, GI:125585934 |
| <400> | 79 |

```
Met Pro Cys Ile Pro Ala Ser Ser Pro Gly Ile Pro His Gln His Gln
  1 5 10 15
His Gln His His Arg Ala Leu Ala Gly Val Gly Met Ala Val Gly Cys
                        20 25 30
Ala Ala Glu Ala Ala Val Ala Ala Ala Gly Val Ala Gly Thr Arg Cys
                35 40 45
Gly Ala His Asp Gly Glu Val Pro Met Glu Val Ala Arg His His Glu
        50 55 60
His Ala Glu Pro Gly Ser Gly Arg Cys Cys Ser Ala Val Val Gln His
65 70 75 80
Val Ala Ala Pro Ala Ala Ala Val Trp Ser Val Val Arg Arg Phe Asp
                                85 90 95
Gln Pro Gln Ala Tyr Lys Arg Phe Val Arg Ser Cys Ala Leu Leu Ala
                        100 105 110
Gly Asp Gly Gly Leu Gly Lys Val Arg Glu Arg Leu Glu Ile Leu Asp
                115 120 125
Asp Glu Ser His Val Leu Ser Phe Arg Val Val Gly Gly Glu His Arg
        130 135 140
Leu Lys Asn Tyr Leu Ser Val Thr Thr Val His Pro Ser Pro Ser Ala
145 150 155 160
Pro Thr Ala Ala Thr Val Val Val Glu Ser Tyr Val Val Asp Val Pro
                                165 170 175
Pro Gly Asn Thr Pro Glu Asp Thr Arg Val Phe Val Asp Thr Ile Val
                        180 185 190
Lys Cys Asn Leu Gln Ser Leu Ala Lys Thr Ala Glu Lys Leu Ala Ala
                195 200 205
Gly Ala Arg Ala Ala Gly Ser
        210 215
```

| | |
|---|---|
| <210> | 80 |
| <211> | 186 |
| <212> | PRT |
| <213> | Rheum australe |
| <220> | |
| <223> | Himalayan rhubarb pathogen-induced protein-like protein, |
| | GenBank Accession No. ACH63237.1, GI:197312913 |
| <400> | 80 |

```
Met Asn Gly Asp Gly Tyr Gly Gly Ser Glu Glu Glu Phe Val Lys Arg
  1 5 10 15
Tyr His Glu His Val Leu Ala Asp His Gln Cys Ser Ser Val Leu Val
                        20 25 30
Glu His Ile Asn Ala Pro Leu His Leu Val Trp Ser Leu Val Arg Ser
                35 40 45
Phe Asp Gln Pro Gln Lys Tyr Lys Pro Phe Val Ser Arg Cys Val Val
        50 55 60
Gln Gly Gly Asp Leu Glu Ile Gly Ser Val Arg Glu Val Asp Val Lys
65 70 75 80
Ser Gly Leu Pro Ala Thr Thr Ser Met Glu Glu Leu Glu Leu Leu Asp
                                85 90 95
Asp Lys Glu His Val Leu Arg Val Lys Phe Val Gly Gly Asp His Arg
                        100 105 110
Leu Lys Asn Tyr Ser Ser Ile Val Ser Leu His Pro Glu Ile Ile Gly
                115 120 125
Gly Arg Ser Gly Thr Met Val Ile Glu Ser Phe Ile Val Asp Ile Ala
        130 135 140
Asp Gly Asn Thr Lys Glu Glu Thr Cys Tyr Phe Ile Glu Ser Leu Ile
145 150 155 160
Asn Cys Asn Leu Lys Ser Leu Ser Cys Val Ser Glu Arg Leu Ala Val
                                165 170 175
Glu Asp Ile Ala Glu Arg Ile Ala Gln Met
                        180 185
```

| | |
|---|---|
| <210> | 81 |
| <211> | 254 |
| <212> | PRT |
| <213> | Oryza sativa |
| <220> | |
| <223> | rice Japonica Group, cultivar Nipponbare, hypothetical |
| | protein, locus tag OsJ_016770, GenBank Accession No. |
| | EAZ33287.1, GI:125593228 |
| <400> | 81 |

```
Met Val Gly Leu Val Gly Gly Gly Gly Trp Arg Val Gly Asp Asp Ala
  1 5 10 15
Ala Gly Gly Gly Gly Gly Gly Ala Val Ala Ala Gly Ala Ala Ala Ala
                        20 25 30
Ala Glu Ala Glu His Met Arg Arg Leu His Ser Gln Gly Pro Arg Arg
                35 40 45
Ala Pro Val Gln Leu Arg Ala Arg Gln Ala His Gln Gly Ser Cys Ser
        50 55 60
Pro Pro Arg Ile Glu Cys Ala Asn Phe Ala Val Phe Leu Ala Ala Arg
65 70 75 80
Asp Pro Lys Ile Val Trp Ser Leu Val Arg Ser Phe Asp Gln Pro Gln
                                85 90 95
Arg Tyr Lys Pro Phe Val Ser Arg Cys Val Val Arg Gly Gly Asp Leu
                        100 105 110
Glu Ile Gly Ser Val Arg Glu Val Asn Val Lys Thr Gly Leu Pro Ala
                115 120 125
Thr Thr Ser Thr Glu Arg Leu Glu Leu Leu Asp Asp Asp Glu His Ile
        130 135 140
Leu Ser Val Lys Phe Val Gly Gly Asp His Arg Leu Arg Asn Tyr Ser
145 150 155 160
Ser Ile Val Thr Val His Pro Glu Ser Ile Asp Gly Arg Pro Gly Thr
                                165 170 175
Leu Val Ile Glu Ser Phe Val Val Asp Val Pro Asp Gly Asn Thr Lys
                        180 185 190
Asp Glu Thr Cys Tyr Phe Val Glu Ala Val Ile Lys Cys Asn Leu Thr
                195 200 205
Ser Leu Ala Glu Met Val Arg Met Ile Ser Leu Val Leu Pro Phe Met
        210 215 220
Leu Val Asp Arg Met Ser Gly Ile Thr Cys Glu Ser His Leu Glu Thr
225 230 235 240
Thr Leu Val Arg Cys Gly Glu Tyr Ala Val Leu Ala His Val
                                245 250
```

| | |
|---|---|
| <210> | 82 |
| <211> | 186 |
| <212> | PRT |
| <213> | Oryza sativa |
| <220> | |
| <223> | rice Japonica Group, cultivar Nipponbare, hypothetical |
| | protein, locus tag OsJ_005784, GenBank Accession No. |
| | EAZ22301.1, GI:125581370 |
| <400> | 82 |

```
Met Glu Pro His Met Glu Arg Ala Leu Arg Glu Ala Val Ala Ser Glu
  1 5 10 15
Ala Glu Arg Arg Glu Leu Glu Gly Val Val Arg Ala His His Thr Gly
                        20 25 30
Trp Asn Ala Pro Leu Ala Ala Val Trp Pro His Arg Ala Arg Val Arg
                35 40 45
Pro Thr Arg Ser Gly Thr Ser Thr Ser Ser Ser Arg Ala Ser Ser Pro
        50 55 60
Pro Gly Asp Gly Ala Thr Val Gly Ser Val Arg Glu Val Ala Val Val
65 70 75 80
Ser Gly Leu Pro Ala Ser Thr Ser Thr Glu Arg Leu Glu Ile Leu Asp
                                85 90 95
Asp Asp Arg His Val Leu Ser Phe Arg Val Val Gly Gly Asp His Arg
                        100 105 110
Leu Arg Asn Tyr Arg Ser Val Thr Ser Val Thr Glu Phe Ser Ser Pro
                115 120 125
Ser Ser Pro Pro Arg Pro Tyr Cys Val Val Val Glu Ser Tyr Val Val
        130 135 140
Asp Val Pro Glu Gly Asn Thr Glu Glu Asp Thr Arg Met Phe Thr Asp
145 150 155 160
Thr Val Val Lys Leu Asn Leu Gln Lys Leu Ala Ala Val Ala Thr Ser
                                165 170 175
Ser Ser Pro Pro Ala Ala Gly Asn His His
                        180 185
```

| | |
|---|---|
| <210> | 83 |
| <211> | 150 |
| <212> | PRT |
| <213> | Oryza sativa |
| <220> | |
| <223> | rice Japonica Group, cultivar Nipponbare, hypothetical |
| | protein, locus tag OsJ_005938, GenBank Accession No. |
| | EAZ22455.1, GI:125581524 |
| <400> | 83 |

```
Met Glu Val Val Trp Ser Ile Val Arg Arg Phe Glu Glu Pro His Ile
  1 5 10 15
Phe Gln Pro Phe Val Arg Gly Cys Thr Met Arg Gly Ser Thr Ser Leu
                        20 25 30
Ala Val Gly Cys Val Arg Glu Val Asp Phe Lys Ser Gly Phe Pro Ala
                35 40 45
Lys Ser Ser Val Glu Arg Leu Glu Ile Leu Asp Asp Lys Glu His Val
        50 55 60
Phe Gly Val Arg Ile Ile Gly Gly Asp His Arg Leu Lys Asn Tyr Ser
65 70 75 80
Ser Val Leu Thr Ala Lys Pro Glu Val Ile Asp Gly Glu Pro Ala Thr
                                85 90 95
Leu Val Ser Glu Ser Phe Val Val Asp Val Pro Glu Gly Asn Thr Ala
                        100 105 110
Asp Glu Thr Arg His Phe Val Glu Phe Leu Ile Arg Cys Asn Leu Arg
                115 120 125
Ser Leu Ala Met Val Ser Gln Arg Leu Leu Leu Ala Gln Gly Asp Leu
        130 135 140
Ala Glu Pro Pro Gly Gln
145 150
```

| | |
|---|---|
| <210> | 84 |
| <211> | 206 |
| <212> | PRT |
| <213> | Oryza sativa |
| <220> | |
| <223> | rice Japonica Group, cultivar Nipponbare, hypothetical |
| | protein, locus tag OsJ_018129, GenBank Accession No. |
| | EAZ34646.1, GI:125594587 |
| <400> | 84 |

```
Met Pro Tyr Thr Ala Pro Arg Pro Ser Pro Pro Gln His Ser Arg Ile
  1 5 10 15
Gly Gly Cys Gly Gly Gly Gly Val Leu Lys Ala Ala Gly Ala Ala Gly
                        20 25 30
His Ala Ala Ser Cys Val Ala Val Pro Ala Glu Val Ala Arg His His
                35 40 45
Glu His Ala Ala Gly Val Gly Gln Cys Cys Ser Ala Val Val Gln Ala
        50 55 60
Ile Ala Ala Pro Val Asp Ala Val Trp Arg Thr Ser Thr Ser Ser Gly
65 70 75 80
Ala Ala Ala Ser Trp Thr Ala Thr Ala Thr Ala Gly Pro Leu Pro Val
                                85 90 95
Gly Ser Val Arg Glu Phe Arg Val Leu Ser Gly Leu Pro Gly Thr Ser
                        100 105 110
Ser Arg Glu Arg Leu Glu Ile Leu Asp Asp Glu Arg Arg Val Leu Ser
                115 120 125
Phe Arg Val Val Gly Gly Glu His Arg Leu Ser Asn Tyr Arg Ser Val
        130 135 140
Thr Thr Val His Glu Thr Ala Ala Gly Ala Ala Ala Ala Val Val Val
145 150 155 160
Glu Ser Tyr Val Val Asp Val Pro His Gly Asn Thr Ala Asp Glu Thr
                                165 170 175
Arg Met Phe Val Asp Thr Ile Val Arg Cys Asn Leu Gln Ser Leu Ala
                        180 185 190
Arg Thr Ala Glu Gln Leu Ala Leu Ala Ala Pro Arg Ala Ala
                195 200 205
```

| | |
|---|---|
| <210> | 85 |
| <211> | 396 |
| <212> | PRT |
| <213> | Vitis vinifera |
| <220> | |
| <223> | wine grape cultivar Pinot Noir hypothetical protein, clone |
| | ENTAV 115, locus tag VITISV_001710, GenBank Accession No. |
| | CAN76441.1, GI:147770961 |
| <220> | |
| <221> | VARIANT |
| <222> | (1)...(396) |
| <223> | Xaa = any amino acid |
| <400> | 85 |

```
Met Pro Ile Ser Ser Leu Pro Phe Ser Leu Tyr Thr Val Thr Pro Asn
  1 5 10 15
Pro Leu Lys Leu Ile Thr Thr His Ala His Ala Phe Thr Pro His Thr
                        20 25 30
His Ile Phe Thr Leu Lys Phe Met Ser His Thr Tyr Cys Pro His Ile
                35 40 45
His His Ile Thr Ser Ile His Tyr Thr His Leu Leu Xaa Pro Ile Pro
        50 55 60
His Met Pro Leu Gln Pro Pro Leu Pro Pro His Pro Ile Leu Pro Ser
65 70 75 80
Met Pro Ala Phe Gln His Leu Tyr Ser Thr Asn Gln His Leu Gln Val
                                85 90 95
Ala Leu Phe Ser Ala Arg Gly Pro Asn Ile Arg Asp Phe Asn Phe Gln
                        100 105 110
Asp Ala Asp Leu Leu Lys Leu Asp Ile Leu Ala Pro Gly Ser Leu Ile
                115 120 125
Trp Ala Ala Trp Ser Pro Asn Gly Thr Asp Glu Ala Asn Tyr Val Gly
        130 135 140
Glu Gly Ser Pro Thr Val Ala Met Ile Ala Lys Arg Gly Pro Arg His
145 150 155 160
Gly Lys Tyr Met Ala Phe Cys Xaa Met Tyr Arg Asp Asn Val Ala Pro
                                165 170 175
Lys Gly Val Asn Xaa Ala Val Ala Thr Val Lys Thr Lys Arg Thr Ile
                        180 185 190
Gln Leu Lys Thr Ser Leu Glu Ile Ala Cys His Tyr Ala Gly Ile Asn
                195 200 205
Ile Ser Gly Ile Asn Gly Glu Val Met Pro Gly Gln Trp Glu Tyr Gln
        210 215 220
Val Gly Pro Gly Gln Cys Ser Ser Leu Leu Ala Gln Arg Val His Val
225 230 235 240
Pro Leu Ser Ala Val Gly Ser Val Val His Arg Phe Asp Lys Pro Gln
                                245 250 255
Arg Tyr Gln His Val Ile Lys Ser Cys Arg Ile Glu Asp Gly Phe Glu
                        260 265 270
Met Arg Met Gly Xaa Leu Arg Asp Val Asn Ile Ile Ser Gly Leu Pro
                275 280 285
Thr Ala Thr Asn Thr Gly Arg Leu Asp Met Gln Asp Asp Glu Arg His
        290 295 300
Val Thr Arg Cys Pro His Gln Arg Gln Ser Glu Ser Lys Tyr Thr Glu
305 310 315 320
Asn Asn Asn Ser Asp Ala Ser Ser Ile Lys Ser Pro Ile Asn Gly Pro
                                325 330 335
Ser Glu His Leu Lys Thr Ala Ala Ser Pro Lys Thr Glu Ser Ile Ile
                        340 345 350
Val Ile Asp Thr Ser Lys Phe Leu Asn Glu Glu Asp Phe Glu Gly Lys
                355 360 365
Asp Glu Thr Ser Ser Ser Asn Gln Val Gln Ile Glu Asp Glu Asn Trp
        370 375 380
Glu Thr Arg Phe Pro Asn Thr Asp Ala Gly Ile Trp
385 390 395
```

| | |
|---|---|
| <210> | 86 |
| <211> | 443 |
| <212> | PRT |
| <213> | Vitis vinifera |
| <220> | |
| <223> | wine grape cuitivar Pinot Noir hypothetical protein, clone |
| | ENTAV 115, locus tag VITISV_014403, GenBank Accession No. |
| | CAN9881.1, GI: 147828564 |
| <220> | |
| <221> | VARIANT |
| <222> | (1)...(443) |
| <223> | Xaa = any amino acid |
| <400> | 86 |

```
Met Pro Ser Ala Xaa Lys Ser Ser Thr Val Pro Leu Ser Leu Xaa Gln
  1 5 10 15
Phe Lys Leu Gly Leu Arg His Gly His Arg Val Ile Pro Trp Gly Asp
                        20 25 30
Leu Asp Ser Leu Ala Met Leu Gln Arg Gln Leu Asp Val Asp Ile Leu
                35 40 45
Val Thr Gly His Thr His Arg Phe Thr Ala Tyr Lys His Glu Gly Gly
        50 55 60
Val Val Ile Asn Pro Gly Ser Ala Thr Gly Ala Phe Gly Ser Ile Thr
65 70 75 80
Tyr Asp Val Asn Pro Ser Phe Val Leu Met Asp Ile Asp Gly Leu Arg
                                85 90 95
Val Val Val Cys Val Tyr Glu Leu Ile Asp Glu Thr Ala Asn Ile Ile
                        100 105 110
Lys Glu Leu His Ala Arg Lys Ile Ser Phe Gly Thr Lys Ser Met Ile
                115 120 125
Xaa Cys Leu Leu Leu Lys Arg Arg Ser Thr Pro Lys Phe Arg Arg Lys
        130 135 140
Lys Leu Phe Leu Phe Gln Cys Arg Val Gln Met Thr Leu Thr Leu Thr
145 150 155 160
Asn Leu Ala Val Ser Gly Ile Ala Gln Thr Leu Gln Val Asp Gln Trp
                                165 170 175
Thr Val Cys Ala Leu Ile Phe Met Thr Arg Arg Asp Ile His Leu Asp
                        180 185 190
Lys Ala Arg Phe Leu Asp Phe Lys Asp Met Gly Lys Leu Leu Ala Asp
                195 200 205
Ala Ser Gly Leu Arg Lys Ala Leu Ser Gly Gly Xaa Val Thr Ala Gly
        210 215 220
Met Ala Ile Phe Asp Thr Met Arg His Ile Arg Pro Asp Val Pro Thr
225 230 235 240
Val Cys Val Gly Leu Ala Ala Val Ala Met Ile Ala Lys Arg Gly Pro
                                245 250 255
Arg His Gly Lys Tyr Met Ala Phe Cys Pro Met Tyr Arg Asp Asn Val
                        260 265 270
Ala Pro Lys Gly Val Asn Val Ala Val Val Thr Val Lys Thr Lys Arg
                275 280 285
Thr Ile Gln Leu Lys Thr Ser Leu Glu Ile Ala Cys His Tyr Ala Gly
        290 295 300
Ile Asn Ile Ser Gly Ile Asn Gly Glu Val Met Pro Gly Gln Trp Glu
305 310 315 320
Tyr Gln Val Gly Pro Gly Gln Cys Ser Ser Leu Leu Ala Gln Arg Val
                                325 330 335
His Val Pro Leu Ser Ala Val Gly Ser Val Val His Arg Phe Asp Lys
                        340 345 350
Pro Gln Arg Tyr Gln His Val Ile Lys Ser Cys Arg Ile Glu Asp Gly
                355 360 365
Phe Glu Met Arg Met Gly Arg Leu Arg Asp Val Asn Ile Ile Ser Gly
        370 375 380
Leu Pro Thr Ala Thr Asn Thr Gly Arg Leu Asp Met Gln Asp Asp Glu
385 390 395 400
Xaa His Val Thr Arg Cys Pro His Gln Arg Gln Ser Glu Ser Lys Tyr
                                405 410 415
Thr Glu Asn Asn Asn Ser Asp Ala Ser Ser Val Lys Ser Pro Ile Asn
                        420 425 430
Gly Pro Ser Glu His Leu Lys Thr Ala Ala Xaa
                435 440
```

| | |
|---|---|
| <210> | 87 |
| <211> | 95 |
| <212> | PRT |
| <213> | Oryza sativa |
| <220> | |
| <223> | rice Indica Group, cultivar Pokkali, capipl protein |
| | (partial), clone OSR-385-428-D5, GenBank Accession No. |
| | ABR25904.1, GI:149392053 |
| <400> | 87 |

```
Glu Ile Gly Ser Val Arg Glu Val Asn Val Lys Thr Gly Leu Pro Ala
  1 5 10 15
Thr Thr Ser Thr Glu Arg Leu Glu Leu Leu Asp Asp Asp Glu His Ile
                        20 25 30
Leu Ser Val Lys Phe Val Gly Gly Asp His Arg Leu Arg Asn Tyr Ser
                35 40 45
Ser Ile Val Thr Val His Pro Glu Ser Ile Asp Gly Arg Pro Gly Thr
        50 55 60
Leu Val Ile Glu Ser Phe Val Val Asp Val Pro Asp Gly Asn Thr Lys
65 70 75 80
Asp Glu Thr Cys Tyr Phe Val Glu Ala Val Ile Lys Cys Asn Leu
                                85 90 95
```

| | |
|---|---|
| <210> | 88 |
| <211> | 191 |
| <212> | PRT |
| <213> | Zea mays |
| <220> | |
| <223> | maize strain B73 unknown protein, clone ZM_BFc0034007, |
| | GenBank Accession No. ACF84624.1, GI:194701080 |
| <400> | 88 |

```
Met Val Val Glu Met Asp Gly Gly Val Gly Val Ala Ala Ala Gly Gly
  1 5 10 15
Gly Gly Ala Gln Thr Pro Ala Pro Pro Pro Pro Arg Arg Trp Arg Leu
                        20 25 30
Ala Asp Glu Arg Cys Asp Leu Arg Ala Met Glu Thr Asp Tyr Val Arg
                35 40 45
Arg Phe His Arg His Glu Pro Arg Asp His Gln Cys Ser Ser Ala Val
        50 55 60
Ala Lys His Ile Lys Ala Pro Val His Leu Val Trp Ser Leu Val Arg
65 70 75 80
Arg Phe Asp Gln Pro Gln Leu Phe Lys Pro Phe Val Ser Arg Cys Glu
                                85 90 95
Met Lys Gly Asn Ile Glu Ile Gly Ser Val Arg Glu Val Asn Val Lys
                        100 105 110
Ser Gly Leu Pro Ala Thr Arg Ser Thr Glu Arg Leu Glu Leu Leu Asp
                115 120 125
Asp Asp Glu Arg Ile Leu Ser Val Arg Phe Val Gly Gly Asp His Arg
        130 135 140
Leu Gln Val Cys Ser Val Leu His Leu Ser Ile Phe Cys Ala Ala His
145 150 155 160
Ala Arg Tyr Phe Ala His His Leu Lys Cys Val Leu Glu Phe Leu Cys
                                165 170 175
Gln Met His Leu Asp Val Leu Pro Cys Asp Asp Ala Ile Leu Glu
                        180 185 190
```

| | |
|---|---|
| <210> | 89 |
| <211> | 239 |
| <212> | PRT |
| <213> | Oryza sativa |
| <220> | |
| <223> | rice Japonica Group, cultivar Nipponbare, hypothetical |
| | protein, locus tag OsJ_020681, GenBank Accession No. |
| | EAZ37198.1, GI:125597418 |
| <400> | 89 |

```
Met Asn Gly Cys Thr Gly Gly Ala Gly Gly Val Ala Ala Gly Arg Leu
  1 5 10 15
Pro Ala Val Ser Leu Gln Gln Ala Gln Trp Lys Leu Val Asp Glu Arg
                        20 25 30
Cys Glu Leu Arg Glu Glu Glu Met Glu Tyr Val Arg Arg Phe His Arg
                35 40 45
His Glu Ile Gly Ser Asn Gln Cys Asn Ser Phe Ile Ala Lys His Val
        50 55 60
Arg Ala Pro Leu Gln Asn Val Trp Ser Leu Val Arg Arg Phe Asp Gln
65 70 75 80
Pro Gln Ile Tyr Lys Pro Phe Val Arg Lys Cys Val Met Arg Gly Asn
                                85 90 95
Val Glu Thr Gly Ser Val Arg Glu Ile Ile Val Gln Ser Gly Leu Pro
                        100 105 110
Ala Thr Arg Ser Ile Glu Arg Leu Glu Phe Leu Asp Asp Asn Glu Tyr
                115 120 125
Ile Leu Arg Val Lys Phe Ile Gly Gly Asp His Met Leu Lys Lys Arg
        130 135 140
Ile Pro Lys Lys Thr Tyr Ala Ile Ser Ser Arg Thr Cys Ser Asp Ser
145 150 155 160
Ala Ile Ile Ala Val Gly Gln Ser Asn Cys Ala Pro Glu Ile Thr Ala
                                165 170 175
Met Asn Gly Gly Val Ser Ile Gln Pro Trp Leu Ile Leu Leu Ala Phe
                        180 185 190
Phe Ser Ser Pro Ser Asn Gln Thr Asn Pro Asp Ser Leu Arg Asp Met
                195 200 205
His Pro Gly Ser Trp Phe Gln Ile Leu Leu Val Leu Ala Met Phe Thr
        210 215 220
Cys Ser Lys Gly Ser Val Leu Pro Pro Ser Glu Lys Val Asn Val
225 230 235
```

| | |
|---|---|
| <210> | 90 |
| <211> | 24 |
| <212> | PRT |
| <213> | Artificial Sequence |
| <220> | |
| <223> | synthetic PYR/PYL polypeptide conserved motif |
| | consensus sequence |
| <220> | |
| <221> | VARIANT |
| <222> | (1)...(24) |
| <223> | Xaa = any amino acid |
| <400> | 90 |

```
Glu Xaa Leu Xaa Xaa Xaa Asp Xaa Xaa Xaa Xaa Xaa Xaa Xaa Xaa Xaa
  1 5 10 15
Xaa Xaa Gly Gly Xaa His Xaa Leu
                        20
```

| | |
|---|---|
| <210> | 91 |
| <211> | 36 |
| <212> | PRT |
| <213> | Artificial Sequence |
| <220> | |
| <223> | synthetic PYR/PYL polypeptide conserved motif |
| | consensus sequence |
| <220> | |
| <221> | VARIANT |
| <222> | (1)...(36) |
| <223> | Xaa = any amino acid |
| <400> | 91 |

```
Cys Xaa Ser Xaa Xaa Xaa Xaa Xaa Xaa Xaa Ala Pro Xaa Xaa Xaa Xaa
  1 5 10 15
Trp Xaa Xaa Xaa Xaa Xaa Phe Xaa Xaa Pro Xaa Xaa Xaa Xaa Xaa Phe
                        20 25 30
Xaa Xaa Xaa Cys
                35
```

| | |
|---|---|
| <210> | 92 |
| <211> | 25 |
| <212> | PRT |
| <213> | Artificial Sequence |
| <220> | |
| <223> | synthetic PYR/PYL polypeptide conserved motif |
| | consensus sequence |
| <220> | |
| <221> | VARIANT |
| <222> | (1)...(25) |
| <223> | Xaa = any amino acid |
| <400> | 92 |

```
Gly Xaa Xaa Arg Xaa Val Xaa Xaa Xaa Ser Xaa Xaa Pro Ala Xaa Xaa
  1 5 10 15
Ser Xaa Glu Xaa Leu Xaa Xaa Xaa Asp
                        20 25
```

| | |
|---|---|
| <210> | 93 |
| <211> | 11 |
| <212> | PRT |
| <213> | Artificial Sequence |
| <220> | |
| <223> | synthetic PYR/PYL polypeptide conserved motif |
| | consensus sequence |
| <220> | |
| <221> | VARIANT |
| <222> | (1)...(11) |
| <223> | Xaa = any amino acid |
| <400> | 93 |

Gly Gly Xaa His Arg Leu Xaa Asn Tyr Xaa Ser
1 5 10

| | |
|---|---|
| <210> | 94 |
| <211> | 36 |
| <212> | PRT |
| <213> | Artificial Sequence |
| <220> | |
| <223> | synthetic PYR1 to PYL12 Arabidopsis PYR/PYL |
| | polypeptide consensus sequence |
| <220> | |
| <221> | VARIANT |
| <222> | (1)...(36) |
| <223> | Xaa = any amino acid |
| <400> | 94 |

```
Cys Xaa Ser Xaa Xaa Xaa Xaa Xaa Xaa Xaa Ala Pro Xaa Xaa Xaa Xaa
  1 5 10 15
Trp Xaa Xaa Xaa Xaa Xaa Phe Xaa Xaa Pro Xaa Xaa Xaa Xaa Xaa Phe
                        20 25 30
Xaa Xaa Xaa Cys
                35
```

| | |
|---|---|
| <210> | 95 |
| <211> | 25 |
| <212> | PRT |
| <213> | Artificial Sequence |
| <220> | |
| <223> | synthetic PYR1 to PYL12 Arabidopsis PYR/PYL |
| | polypeptide consensus sequence |
| <220> | |
| <221> | VARIANT |
| <222> | (1)...(25) |
| <223> | Xaa = any amino acid |
| <400> | 95 |

```
Gly Xaa Xaa Arg Xaa Val Xaa Xaa Xaa Ser Xaa Xaa Pro Ala Xaa Xaa
  1 5 10 15
Ser Xaa Glu Xaa Leu Xaa Xaa Xaa Asp
                        20 25
```

| | |
|---|---|
| <210> | 96 |
| <211> | 31 |
| <212> | PRT |
| <213> | Artificial Sequence |
| <220> | |
| <223> | synthetic PYR1 to PYL12 Arabidopsis PYR/PYL |
| | polypeptide consensus sequence |
| <220> | |
| <221> | VARIANT |
| <222> | (1)...(31) |
| <223> | Xaa = any amino acid |
| <400> | 96 |

```
Glu Ser Xaa Xaa Val Asp Xaa Pro Xaa Gly Xaa Xaa Xaa Xaa Xaa Thr
  1 5 10 15
Xaa Xaa Phe Xaa Xaa Xaa Xaa Xaa Xaa Xaa Asn Leu Xaa Xaa Leu
                        20 25 30
```

| | |
|---|---|
| <210> | 97 |
| <211> | 36 |
| <212> | PRT |
| <213> | Artificial Sequence |
| <220> | |
| <223> | synthetic PYL1-12 Arabidopsis PYR/PYL polypeptide |
| | consensus sequence |
| <220> | |
| <221> | VARIANT |
| <222> | (1)...(36) |
| <223> | Artificial Sequence |
| <400> | 97 |

```
Cys Xaa Ser Xaa Xaa Xaa Xaa Xaa Xaa Xaa Ala Pro Xaa Xaa Xaa Xaa
  1 5 10 15
Trp Xaa Xaa Xaa Xaa Xaa Phe Xaa Xaa Pro Xaa Xaa Xaa Lys Xaa Phe
                        20 25 30
Xaa Xaa Xaa Cys
                35
```

| | |
|---|---|
| <210> | 98 |
| <211> | 25 |
| <212> | PRT |
| <213> | Artificial Sequence |
| <220> | |
| <223> | synthetic PYL1-12 Arabidopsis PYR/PYL polypeptide |
| | consensus sequence |
| <220> | |
| <221> | VARIANT |
| <222> | (1)...(25) |
| <223> | Xaa = any amino acid |
| <400> | 98 |

```
Gly Xaa Xaa Arg Xaa Val Xaa Xaa Xaa Ser Xaa Leu Pro Ala Xaa Xaa
  1 5 10 15
Ser Xaa Glu Xaa Leu Xaa Xaa Xaa Asp
                        20 25
```

| | |
|---|---|
| <210> | 99 |
| <211> | 31 |
| <212> | PRT |
| <213> | Artificial Sequence |
| <220> | |
| <223> | synthetic PYL1-12 Arabidopsis PYR/PYL polypeptide |
| | consensus sequence |
| <220> | |
| <221> | VARIANT |
| <222> | (1)...(31) |
| <223> | Xaa = any amino acid |
| <400> | 99 |

```
Glu Ser Xaa Xaa Val Asp Xaa Pro Xaa Gly Asn Xaa Xaa Xaa Xaa Thr
  1 5 10 15
Xaa Xaa Phe Xaa Xaa Xaa Xaa Xaa Xaa Xaa Asn Leu Xaa Xaa Leu
                        20 25 30
```

| | |
|---|---|
| <210> | 100 |
| <211> | 45 |
| <212> | PRT |
| <213> | Artificial Sequence |
| <220> | |
| <223> | synthetic PYL1-6 Arabidopsis PYR/PYL polypeptide |
| | consensus sequence |
| <220> | |
| <221> | VARIANT |
| <222> | (1)...(45) |
| <223> | Xaa = any amino acid |
| <400> | 100 |

```
His Xaa Xaa Xaa Xaa Xaa Xaa Xaa Xaa Cys Xaa Ser Xaa Xaa Xaa Xaa
  1 5 10 15
Xaa Xaa Xaa Ala Pro Xaa Xaa Xaa Xaa Trp Xaa Xaa Xaa Xaa Xaa Phe
                        20 25 30
Xaa Xaa Pro Xaa Xaa Tyr Lys Xaa Phe Xaa Xaa Xaa Cys
                35 40 45
```

| | |
|---|---|
| <210> | 101 |
| <211> | 50 |
| <212> | PRT |
| <213> | Artificial Sequence |
| <220> | |
| <223> | synthetic PYL1-6 Arabidopsis PYR/PYL polypeptide |
| | consensus sequence |
| <220> | |
| <221> | VARIANT |
| <222> | (1)...(50) |
| <223> | Xaa = any amino acid |
| <400> | 101 |

```
Val Gly Xaa Xaa Arg Xaa Val Xaa Val Xaa Ser Gly Leu Pro Ala Xaa
  1 5 10 15
Xaa Ser Xaa Glu Xaa Leu Xaa Xaa Xaa Asp Xaa Xaa Xaa Xaa Xaa Xaa
                        20 25 30
Xaa Phe Xaa Xaa Xaa Gly Gly Xaa His Arg Leu Xaa Asn Tyr Xaa Ser
                35 40 45
Val Thr
        50
```

| | |
|---|---|
| <210> | 102 |
| <211> | 33 |
| <212> | PRT |
| <213> | Artificial Sequence |
| <220> | |
| <223> | synthetic PYL1-6 Arabidopsis PYR/PYL polypeptide |
| | consensus sequence |
| <220> | |
| <221> | VARIANT |
| <222> | (1)...(33) |
| <223> | Xaa = any amino acid |
| <400> | 102 |

```
Val Xaa Glu Ser Tyr Xaa Val Asp Xaa Pro Xaa Gly Asn Xaa Xaa Xaa
  1 5 10 15
Xaa Thr Xaa Xaa Phe Xaa Asp Xaa Xaa Xaa Xaa Xaa Asn Leu Gln Xaa
                        20 25 30
Leu
```

| | |
|---|---|
| <210> | 103 |
| <211> | 50 |
| <212> | PRT |
| <213> | Artificial Sequence |
| <220> | |
| <223> | synthetic PYL7-10 Arabidopsis PYR/PYL polypeptide |
| | consensus sequence |
| <220> | |
| <221> | VARIANT |
| <222> | (1)...(50) |
| <223> | Xaa = any amino acid |
| <400> | 103 |

```
His Xaa His Xaa Xaa Xaa Xaa Xaa Gln Cys Xaa Ser Xaa Leu Val Lys
  1 5 10 15
Xaa Ile Xaa Ala Pro Xaa His Xaa Val Trp Ser Xaa Val Arg Arg Phe
                        20 25 30
Asp Xaa Pro Gln Lys Tyr Lys Pro Phe Xaa Ser Arg Cys Xaa Val Xaa
                35 40 45
Gly Xaa
        50
```

| | |
|---|---|
| <210> | 104 |
| <211> | 61 |
| <212> | PRT |
| <213> | Artificial Sequence |
| <220> | |
| <223> | synthetic PYL7-10 Arabidopsis PYR/PYL polypeptide |
| | consensus sequence |
| <220> | |
| <221> | VARIANT |
| <222> | (1)...(61) |
| <223> | Xaa = any amino acid |
| <400> | 104 |

```
Glu Xaa Gly Xaa Xaa Arg Glu Val Xaa Xaa Lys Ser Gly Leu Pro Ala
  1 5 10 15
Thr Xaa Ser Thr Glu Xaa Leu Glu Xaa Leu Asp Asp Xaa Glu His Ile
                        20 25 30
Leu Xaa Ile Xaa Ile Xaa Gly Gly Asp His Arg Leu Lys Asn Tyr Ser
                35 40 45
Ser Xaa Xaa Xaa Xaa His Xaa Glu Xaa Ile Xaa Gly Xaa
        50 55 60
```

| | |
|---|---|
| <210> | 105 |
| <211> | 44 |
| <212> | PRT |
| <213> | Artificial Sequence |
| <220> | |
| <223> | synthetic PYL7-10 Arabidopsis PYR/PYL polypeptide |
| | consensus sequence |
| <220> | |
| <221> | VARIANT |
| <222> | (1)...(44) |
| <223> | Xaa = any amino acid |
| <400> | 105 |

```
Xaa Gly Thr Xaa Xaa Xaa Glu Ser Phe Val Val Asp Val Pro Xaa Gly
  1 5 10 15
Asn Thr Lys Xaa Xaa Thr Cys Xaa Phe Val Glu Xaa Leu Ile Xaa Cys
                        20 25 30
Asn Leu Xaa Ser Leu Ala Xaa Xaa Xaa Glu Arg Leu
                35 40
```

| | |
|---|---|
| <210> | 106 |
| <211> | 44 |
| <212> | PRT |
| <213> | Artificial Sequence |
| <220> | |
| <223> | synthetic PYL11-13 Arabidopsis PYR/PYL polypeptide |
| | consensus sequence |
| <220> | |
| <221> | VARIANT |
| <222> | (1)...(44) |
| <223> | Xaa = any amino acid |
| <400> | 106 |

```
Cys Xaa Ser Xaa Xaa Val Xaa Thr Ile Xaa Ala Pro Leu Xaa Leu Val
  1 5 10 15
Trp Ser Ile Leu Arg Xaa Phe Asp Xaa Pro Xaa Xaa Xaa Xaa Xaa Phe
                        20 25 30
Val Lys Xaa Cys Xaa Xaa Xaa Ser Gly Xaa Gly Gly
                35 40
```

| | |
|---|---|
| <210> | 107 |
| <211> | 49 |
| <212> | PRT |
| <213> | Artificial Sequence |
| <220> | |
| <223> | synthetic PYL11-13 Arabidopsis PYR/PYL polypeptide |
| | consensus sequence |
| <220> | |
| <221> | VARIANT |
| <222> | (1)...(49) |
| <223> | Xaa = any amino acid |
| <400> | 107 |

```
Gly Ser Val Arg Xaa Val Thr Xaa Val Ser Xaa Xaa Pro Ala Xaa Phe
  1 5 10 15
Ser Xaa Glu Arg Leu Xaa Glu Leu Asp Asp Glu Ser His Val Met Xaa
                        20 25 30
Xaa Ser Ile Ile Gly Gly Xaa His Arg Leu Val Asn Tyr Xaa Ser Lys
                35 40 45
Thr
```

| | |
|---|---|
| <210> | 108 |
| <211> | 40 |
| <212> | PRT |
| <213> | Artificial Sequence |
| <220> | |
| <223> | synthetic PYL11-13 Arabidopsis PYR/PYL polypeptide |
| | consensus sequence |
| <220> | |
| <221> | VARIANT |
| <222> | (1)...(40) |
| <223> | Xaa = any amino acid |
| <400> | 108 |

```
Lys Lys Thr Val Val Val Glu Ser Tyr Val Val Asp Val Pro Glu Gly
  1 5 10 15
Xaa Xaa Glu Glu Xaa Thr Xaa Xaa Phe Xaa Asp Xaa Ile Xaa Xaa Xaa
                        20 25 30
Asn Leu Xaa Ser Leu Ala Lys Leu
                35 40
```

1. A protein dimer comprising a first amino acid sequence and a second amino acid sequence, wherein the protein dimer dissociates in the presence of a plant hormone and the dissociation results in a detectable signal.

2. The protein dimer of claim 1, wherein the plant hormone is abscisic acid (ABA).

3. The protein dimer of claim 1, wherein the protein dimer is a heterodimer.

4. The protein dimer of claim 1, wherein the protein dimer is a homodimer.

5. The protein dimer of claim 1, wherein one or more of the first and second amino acid sequences is a PYL protein.

6. The protein dimer of claim 5, wherein the PYL protein is not covalently linked to a phosphatase.

7. The protein dimer of claim 5, wherein the PYL protein is a PYL3 protein.

8. The protein dimer of claim 1, wherein the first amino acid sequence comprises a fluorescent protein sequence and the second amino acid sequence comprises a first quencher protein sequence.

9. The protein dimer of claim 1, wherein the first amino acid sequence is conjugated to a first dye molecule and the second amino acid sequence is conjugated to a first quencher.

10. The protein dimer of claim 9, wherein the first quencher is also a dye molecule that emits a detectable signal.

11. The protein dimer of claim 10, wherein the first dye molecule is also a quencher with respect to the detectable signal of the first quencher.

12. The protein dimer of claim 2, wherein the detectable signal is fluorescent or colorimetric.

13. (canceled)

14. A plant comprising one or more exogenous genes encoding the first and second amino acid sequences of claim 1.

15. A plant expressing the first and second amino acid sequences of claim 1.

16. A method of monitoring plant hormones in a plurality of adjacent plants wherein at least one plant in the plurality is a plant comprising or expressing the first and second amino acid sequences of claim 1, the method comprising
detecting the detectable signal from the at least one plant in the plurality.

17. The method of claim 16, wherein the first amino acid sequence and the second amino acid sequence are injected into the plant.

18. The method of claim 17, wherein the first and second amino acid sequences are identical and linked to a self-quenching fluorescent label.

19. The method of claim 16, further comprising altering at least one environmental condition of the plurality if the level of detectable signal exceeds or is below a threshold value.

20. The method of claim 19, wherein the altering comprises providing the plurality with water or nutrients or pesticides.

21. The method of claim 16, wherein the detecting is performed by a detector over the plurality of plants.

22. (canceled)
